(19) United States
(12) Patent Application Publication
(10) Pub. No.: US 2023/0183309 A1
(43) Pub. Date: Jun. 15, 2023

(54) STRUCTURALLY-STABILIZED GLUCAGON-LIKE PEPTIDE 1 PEPTIDES AND USES THEREOF

(71) Applicant: Dana-Farber Cancer Institute, Inc., Boston, MA (US)

(72) Inventors: Loren D. Walensky, Newton, MA (US); Gregory H. Bird, Pelham, NH (US)

(21) Appl. No.: 17/786,157

(22) PCT Filed: Dec. 18, 2020
(86) PCT No.: PCT/US2020/066094

§ 371 Date: Jun. 16, 2022

Related U.S. Application Data
(60) Provisional application No. 62/951,503, filed on Dec. 20, 2019.

(51) Int. Cl. C07K 14/605; A61P 3/10; A61K 9/00

(52) U.S. Cl. CPC C07K 14/605; A61K 9/0019; A61P 3/10

(57) ABSTRACT

This disclosure features structurally-stabilized peptides that target glucagon-like peptide 1 receptor (GLP-1R), compositions comprising same, and methods for using such peptides in the treatment of diabetes, hyperglycemia, cardiovascular disease, obesity, Alzheimer’s disease, Huntington’s disease, and other conditions that can benefit from increased GLP-1 agonist activity and in increasing cAMP levels

### CROSS-REFERENCE TO RELATED APPLICATIONS

This application claims the priority benefit of U.S. Provisional Application No. 62/951,503, filed Dec. 20, 2019, which is hereby incorporated by reference herein in its entirety.

### SEQUENCE LISTING

The instant application contains a Sequence Listing which has been submitted electronically in ASCII format and is hereby incorporated by reference in its entirety. Said ASCII copy, created on Dec. 18, 2020, is named 00530-0359WO1_SL.txt and is 67,045 bytes in size.

### TECHNICAL FIELD

This disclosure relates to structurally-stabilized peptides that target glucagon-like peptide 1 receptor (GLP-1R), compositions comprising same, and methods for using such peptides in the treatment of type 1 and/or 2 diabetes or hyperglycemia, with effects on improving blood glucose control, preserving beta-cell function, delaying gastric emptying, enabling weight loss, increasing insulin sensitivity, and mitigating cardiovascular disease, and other conditions that can benefit from increased GLP-1 agonist activity and in increasing cAMP levels. The disclosure also relates to using the peptides in the treatment of Alzheimer’s and Huntington’s disease.

### BACKGROUND

Diabetes refers to a disease process resulting in abnormal glucose homeostasis that is derived from multiple causative factors and characterized by elevated levels of glucose in the blood (i.e., hyperglycemia). Persistent or uncontrolled hyperglycemia is associated with increased and premature morbidity and mortality. Often abnormal glucose homeostasis is also associated both directly and indirectly with alterations of the lipid, lipoprotein and apolipoprotein metabolism and other metabolic and hemodynamic diseases. Therefore, patients with type 2 diabetes mellitus are at especially increased risk of macrovascular and microvascular complications, including coronary heart disease, stroke, peripheral vascular disease, hypertension, nephropathy, neuropathy, and retinopathy. Therefore, therapeutic control of glucose homeostasis, lipid metabolism and hypertension are critically important in the clinical management and treatment of diabetes mellitus.

There are two generally recognized forms of diabetes. In type 1 diabetes, or insulin-dependent diabetes mellitus (IDDM), patients produce little or no insulin, the hormone which regulates glucose utilization. In type 2 diabetes, or noninsulin dependent diabetes mellitus (NIDDM), patients often have plasma insulin levels that are the same or even elevated compared to nondiabetic subjects; however, these patients have developed a resistance to the insulin stimulating effect on glucose and lipid metabolism in the main insulin-sensitive tissues, which are muscle, liver and adipose tissues, and the plasma insulin levels, while elevated, are insufficient to overcome the pronounced insulin resistance.

Insulin resistance is not primarily due to a diminished number of insulin receptors but to a post-insulin receptor binding defect that is not yet understood. This resistance to insulin responsiveness results in insufficient insulin activation of glucose uptake, oxidation and storage in muscle and inadequate insulin repression of lipolysis in adipose tissue and of glucose production and secretion in the liver.

The available treatments for type 1 and 2 diabetes have recognized limitations. Accordingly, there is a need for new treatments for diabetes and hyperglycemia.

GLP-1 is also produced in the central nervous system. Hyperinsulinemia and insulin resistance have been demonstrated to have significant impact on cognitive impairment. The most prevalent form of cognitive impairment is Alzheimer’s disease. However, the available treatments for Alzheimer’s disease have recognized limitations. Accordingly, there is a need for new treatments for Alzheimer’s disease.

Huntington’s disease is a fatal neurodegenerative disease. Studies have shown that the prevalence of type-2 diabetes is higher in patients with Huntington’s disease than in healthy controls. The available treatments for Huntington’s disease have recognized limitations. Accordingly, there is a need for new treatments for Huntington’s disease.

### SUMMARY

Glucagon-like peptide 1 (GLP-1) is a natural peptide agonist of the GLP-1 receptor (GLP-1R) found on pancreatic β-cells. Engagement of its receptor stimulates insulin release in a glucose-dependent fashion and increases β-cell mass, two ideal features for pharmacologic management of diabetes. Thus, intensive efforts have focused on developing GLP-1-based peptide agonists of GLP-1R for therapeutic application. A primary challenge has been the naturally short half-life of GLP-1 due to its rapid proteolytic degradation in vivo. This disclosure describes the development of a unique approach to preserving the structure and function of GLP-1 by all-hydrocarbon i, i+7 stitching. The “stitch” is especially well-suited for reinforcing and protecting the particular structure-activity relationships of GLP-1. The stitched GLP-1 peptides described herein demonstrated potent biological activity and striking proteolytic stability in vitro and in vivo. This disclosure also features methods for using such stitched peptides alone or in combination with other therapeutic agents in the treatment of diabetes and/or hyperglycemia. This disclosure also features methods for using such stitched peptides alone or in combination with other therapeutic agents in the treatment of Alzheimer’s disease and Huntington’s disease. The disclosure also features compositions comprising such stitched peptides and methods of making the stitched peptides.

Provided herein is a peptide comprising the amino acid sequence (i) HJEGTFTSD8SSYLEG#AAKEFIZWLVKGR set forth in SEQ ID NO:40; or (ii) HGEGTFTSD8SSYLEG#AAKEFIZWLVKGR set forth in SEQ ID NO:33, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine, and J is 2-aminoisobutyric acid, and wherein the peptide binds to glucagon-like peptide 1 receptor (SEQ ID NO:5). This disclosure also features a peptide comprising the amino acid sequence set forth in SEQ ID NO: 33 or 40, having 1 to 25 (e.g., 1, 2, 3, 4, 5, 6, 7, 8, 9, 10, 11, 12, 13, 14, 15, 16, 17, 18, 19, 20, 21, 22, 23, 24, 25) amino acid substitutions, wherein the peptide binds to glucagon-like peptide 1 receptor (SEQ ID NO:5). In certain instances, the substitutions are not at positions 2, 10, 17 or 24 of SEQ ID NO: 33 or 40. In certain instances, the substitutions are not at positions in the N-terminal portion (i.e., amino acids corresponding to positions 6-15 of SEQ ID NO:2) of the GLP-1 peptide. In some instances, the substitutions are not at positions 2, 10, 17 or 24 of SEQ ID NO: 33 or 40 and not at positions in the N-terminal portion (i.e., amino acids corresponding to positions 6-15 of SEQ ID NO:2) of the GLP-1 peptide. In certain instances, the substitutions are not at positions on the GLP-1R interacting face of the C-terminal portion (i.e., amino acids corresponding to positions 16-37 of SEQ ID NO:2) of the GLP-1 peptide. In some instances, the substitutions are not at positions 2, 10, 17 or 24 of SEQ ID NO: 33 or 40 and not at any of the positions on the GLP-1R interacting face of the C-terminal portion (i.e., amino acids corresponding to positions 16-37 of SEQ ID NO:2) of the GLP-1 peptide. In certain instances, the substitutions are at one or more (e.g., 1, 2, 3, 4, 5, 6, 7, 8, 9, 10, 11, 12, 13, 14, 15, 16, 17, 18, 19, 20, 21) positions on the non-GLP-1R interacting face of the C-terminal portion (i.e., amino acids corresponding to positions 16-37 of SEQ ID NO:2)of the GLP-1 peptide. In certain instances, the substitutions are at one or more (e.g., 1, 2, 3, 4, 5, 6, 7, 8, 9) positions on the GLP-1R interacting face of the C-terminal portion (i.e., amino acids corresponding to positions 16-37 of SEQ ID NO:2) of the GLP-1 peptide and at one or more (e.g., 1, 2, 3, 4, 5, 6, 7, 8, 9, 10, 11, 12, 13, 14, 15, 16, 17, 18, 19, 20, 21) positions on the non-GLP-1R interacting face of the C-terminal portion (i.e., amino acids corresponding to positions 16-37 of SEQ ID NO:2) of the GLP-1 peptide. In some instances, the substitutions are conservative amino acid substitutions. In other instances, the substitutions are non-conservative amino acid substitutions.

Also provided herein is a stitched peptide comprising the amino acid sequence (i) HJEGTFTSD8SSYLEG#AAKEFIZWLVKGR set forth in SEQ ID NO:40; or (ii) HGEGTFTSD8SSYLEG#AAKEFIZWLVKGR set forth in SEQ ID NO:33, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine, and J is 2-aminoisobutyric acid, and wherein the peptide binds to glucagon-like peptide 1 receptor (SEQ ID NO:5). This disclosure also features a stitched peptide comprising the amino acid sequence set forth in SEQ ID NO: 33 or 40, having 1 to 25 (e.g., 1, 2, 3, 4, 5, 6, 7, 8, 9, 10, 11, 12, 13, 14, 15, 16, 17, 18, 19, 20, 21, 22, 23, 24, 25) amino acid substitutions, wherein the peptide binds to glucagon-like peptide 1 receptor (SEQ ID NO:5). In certain instances, the substitutions are not at positions in the N-terminal portion (i.e., amino acids corresponding to positions 6-15 of SEQ ID NO:2) of the GLP-1 peptide. In some instances, the substitutions are not at positions 2, 10, 17 or 24 of SEQ ID NO: 33 or 40 and not at positions in the N-terminal portion (i.e., amino acids corresponding to positions 6-15 of SEQ ID NO:2) of the GLP-1 peptide. In certain instances, the substitutions are not at positions on the GLP-1R interacting face of the C-terminal portion (i.e., amino acids corresponding to positions 16-37 of SEQ ID NO:2) of the GLP-1 peptide. In some instances, the substitutions are not at positions 2, 10, 17 or 24 of SEQ ID NO: 33 or 40 and not at any of the positions on the GLP-1R interacting face of the C-terminal portion (i.e., amino acids corresponding to positions 16-37 of SEQ ID NO:2) of the GLP-1 peptide. In certain instances, the substitutions are at one or more (e.g., 1, 2, 3, 4, 5, 6, 7, 8, 9, 10, 11, 12, 13, 14, 15, 16, 17, 18, 19, 20, 21) positions on the non-GLP-1R interacting face of the C-terminal portion (i.e., amino acids corresponding to positions 16-37 of SEQ ID NO:2) of the GLP-1 peptide. In certain instances, the substitutions are at one or more (e.g., 1, 2, 3, 4, 5, 6, 7, 8, 9) positions on the GLP-1R interacting face of the C-terminal portion (i.e., amino acids corresponding to positions 16-37 of SEQ ID NO:2) of the GLP-1 peptide and at one or more (e.g., 1, 2, 3, 4, 5, 6, 7, 8, 9, 10, 11, 12, 13, 14, 15, 16, 17, 18, 19, 20, 21) positions on the non-GLP-1R interacting face of the C-terminal portion (i.e., amino acids corresponding to positions 16-37 of SEQ ID NO:2) of the GLP-1 peptide. In some instances, the substitutions are conservative amino acid substitutions. In other instances, the substitutions are non-conservative amino acid substitutions.

Also provided herein is a stitched peptide comprising the amino acid sequence (i) HJEGTFTSDVSSYLEGQAAKEFIAWLVKGR set forth in SEQ ID NO:38, wherein J is 2-aminoisobutyric acid; or (ii) HGEGTFTSDVSSYLEGQAAKEFIAWLVKGR set forth in SEQ ID NO:31, wherein each of positions 10, 17, and 24 of the amino acid sequence of SEQ ID NO:38 or 31 is replaced with a stapling amino acid, wherein a sidechain of the stapling amino acid at position 10 is cross-linked to a sidechain of the stapling amino acid at position 17 and a sidechain of the stapling amino acid at position 17 is cross-linked to a side chain of the stapling amino acid at position 24, and wherein the peptide binds to glucagon-like peptide 1 receptor (SEQ ID NO:5). This disclosure also features a stitched peptide comprising the amino acid sequence set forth in SEQ ID NO: 31 or 38, having 1 to 25 (e.g., 1, 2, 3, 4, 5, 6, 7, 8, 9, 10, 11, 12, 13, 14, 15, 16, 17, 18, 19, 20, 21, 22, 23, 24, 25) additional amino acid substitutions (i.e., in addition to the substitutions at positions 10, 17, and 24 with a stapling amino acid), wherein the peptide binds to glucagon-like peptide 1 receptor (SEQ ID NO:5). In certain instances, the additional substitutions are not at positions in the N-terminal portion (i.e., amino acids corresponding to positions 6-15 of SEQ ID NO:2) of the GLP-1 peptide. In some instances, the additional substitutions are not at position 2 of SEQ ID NO:38 or 31 and not at positions in the N-terminal portion (i.e., amino acids corresponding to positions 6-15 of SEQ ID NO:2) of the GLP-1 peptide. In certain instances, the additional substitutions are not at positions on the GLP-1R interacting face of the C-terminal portion (i.e., amino acids corresponding to positions 16-37 of SEQ ID NO:2) of the GLP-1 peptide. In some instances, the additional substitutions are not at position 2 of SEQ ID NO: 31 or 38 and not at any of the positions on the GLP-1R interacting face of the C-terminal portion (i.e., amino acids corresponding to positions 16-37 of SEQ ID NO:2) of the GLP-1 peptide. In certain instances, the additional substitutions are at one or more (e.g., 1, 2, 3, 4, 5, 6, 7, 8, 9, 10, 11, 12, 13, 14, 15, 16, 17, 18, 19, 20, 21) positions on the non-GLP-1R interacting face of the C-terminal portion (i.e., amino acids corresponding to positions 16-37 of SEQ ID NO:2) of the GLP-1 peptide. In certain instances, the additional substitutions are at one or more (e.g., 1, 2, 3, 4, 5, 6, 7, 8, 9) positions on the GLP-1R interacting face of the C-terminal portion (i.e., amino acids corresponding to positions 16-37 of SEQ ID NO:2) of the GLP-1 peptide and at one or more (e.g., 1, 2, 3, 4, 5, 6, 7, 8, 9, 10, 11, 12, 13, 14, 15, 16, 17, 18, 19, 20, 21) positions on the non-GLP-1R interacting face of the C-terminal portion (i.e., amino acids corresponding to positions 16-37 of SEQ ID NO:2) of the GLP-1 peptide. In some instances, the substitutions are conservative amino acid substitutions. In other instances, the substitutions are non-conservative amino acid substitutions.

Also provided herein is a stitched peptide comprising a stitched amino acid sequence having the formula:

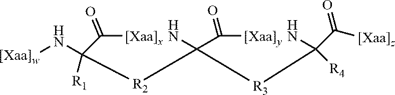

Formula (I) or a pharmaceutically acceptable salt thereof, wherein:
[Xaa]_{w} is HJEGTFTSD (SEQ ID NO:45) or HGEGTFTSD (SEQ ID NO:49),
[Xaa]ₓ is SSYLEG (SEQ ID NO:46),
[Xaa]_{y} is AAKEFI (SEQ ID NO:47),
[Xaa]_{z} is WLVKGR (SEQ ID NO:48),
each R₁ and R₄ is independently H, alkyl, alkenyl, alkynyl, arylalkyl, cycloalkylalkyl, heteroarylalkyl, or heterocyclylalkyl, any of which is substituted or unsubstituted;
each R₂ and R₃ is independently alkylene, alkenylene, or alkynylene, any of which is substituted or unsubstituted;
wherein the stitched amino acid sequence binds to glucagon-like peptide 1 receptor (SEQ ID NO:5), and
wherein the cross-linked amino acid sequence has an alpha helical conformation. In some instances, R₁ is an alkyl. In some instances, R₁ is a methyl group. In some instances, R₄ is an alkyl. In some instances, R₄ is a methyl group. In some instances,
R₂ is an alkenyl. In some instances, R₃ is an alkenyl. In some instances, R₁ is a methyl group, R₂ is (CH₂)₆—CH=CH—(CH₂)₃, R₃ is (CH₂)₃—CH=CH—(CH₂)₆, and R₄ is a methyl group. In some instances, R₁ is a methyl group, R₂ is (CH₂)₃—CH=CH—(CH₂)₆, R₃ is (CH₂)₆—CH=CH—(CH₂)₃, and R₄ is a methyl group. In some instances, the stitched amino acid sequence comprises

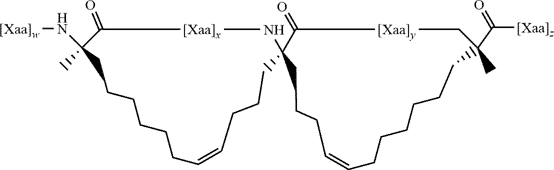

, wherein [Xaa]_{w} is HJEGTFTSD (SEQ ID NO:45) or HGEGTFTSD (SEQ ID NO:49), [Xaa]ₓ is SSYLEG (SEQ ID NO:46), [Xaa]_{y} is AAKEFI (SEQ ID NO:47), and [Xaa]_{z} is WLVKGR (SEQ ID NO:48). In some instances, the pharmaceutically acceptable salt is an acetate, a sulfate, or a chloride.

In some instances, the peptide or stitched peptide described herein is 30 to 50 amino acids in length.

Also provided herein is a stitched peptide comprising a modified amino acid sequence of the sequence set forth in SEQ ID NO:38, wherein the peptide comprises a stitch between amino acids corresponding to positions 10, 17, and 24 of the amino acid sequence set forth in SEQ ID NO:38, and wherein the peptide binds to glucagon-like peptide 1 receptor (SEQ ID NO:5).

Also provided herein is a peptide comprising the amino acid sequence of any one of SEQ ID NOs: 61, 62, 65, 66, 71, 73, 79, 81, 67, 68, 75, 77, 83, and 85, wherein the peptide binds to glucagon-like peptide 1 receptor (SEQ ID NO:5).

Also provided herein is a stitched peptide comprising the amino acid sequence of any one of SEQ ID NOs: 34, 41, 59-68, 71, 73, 75, 77, 79, 81, 83, and 85, wherein the stitched peptide binds to glucagon-like peptide 1 receptor (SEQ ID NO:5).

Also provided herein is a stitched peptide comprising the amino acid sequence (i) HJEGTFTSDVSSYLEGQAAKEFIAWLVKGR set forth in SEQ ID NO:38, wherein J is 2-aminoisobutyric acid; or (ii) HGEGTFTSDVSSYLEGQAAKEFIAWLVKGR set forth in SEQ ID NO:31, wherein (a) each of positions 11, 18, and 25 of SEQ ID NO:38 or 31 is replaced with a stapling amino acid, wherein a sidechain of the stapling amino acid at position 11 is cross-linked to a sidechain of the stapling amino acid at position 18 and a sidechain of the stapling amino acid at position 18 is cross-linked to a side chain of the stapling amino acid at position 25, (b) each of positions 12, 19, and 26 of SEQ ID NO:38 or 31 is replaced with a stapling amino acid, wherein a sidechain of the stapling amino acid at position 12 is cross-linked to a sidechain of the stapling amino acid at position 19 and a sidechain of the stapling amino acid at position 19 is cross-linked to a side chain of the stapling amino acid at position 26, or (c) each of positions 6, 13, and 20 of SEQ ID NO:38 or 31 is replaced with a stapling amino acid, wherein a sidechain of the stapling amino acid at position 6 is cross-linked to a sidechain of the stapling amino acid at position 13 and a sidechain of the stapling amino acid at position 13 is cross-linked to a side chain of the stapling amino acid at position 20, and wherein the stitched peptide binds to glucagon-like peptide 1 receptor (SEQ ID NO:5).

Also provided herein is a stitched peptide comprising a stitched amino acid sequence having the formula:

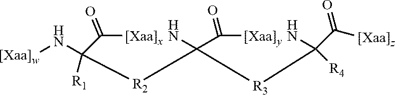

or a pharmaceutically acceptable salt thereof, wherein:
(a) [Xaa]_{w} HJEGTFTSDV (SEQ ID NO:50) or HGEGTFTSDV (SEQ ID NO:54),
   [Xaa]ₓ is SYLEGQ (SEQ ID NO:51),
   [Xaa]_{y} is AKEFIA (SEQ ID NO:52),
   [Xaa]_{z} is LVKGR (SEQ ID NO:53) or LVKGRG (SEQ ID NO:56),
(b) [Xaa]_{w} HGEGTFTSDVS (SEQ ID NO:95) or HJEGTFTSDVS (SEQ ID NO:96),
   [Xaa]ₓ is YLEGQA (SEQ ID NO:89),
   [Xaa]_{y} is KEFIAW (SEQ ID NO:90),
   [Xaa]_{z} is VKGR (SEQ ID NO:97) or VKGRG (SEQ ID NO:98), or
(c) [Xaa]_{w} is HGEGT (SEQ ID NO:91) or HJEGT (SEQ ID NO:92),
   [Xaa]ₓ is TSDVSS (SEQ ID NO:87),
   [Xaa]_{y} is LEGQAA (SEQ ID NO:88),
   [Xaa]_{z} is EFIAWLVKGR (SEQ ID NO:93) or EFIAWLVKGRG (SEQ ID NO:94),
   each R₁ and R₄ is independently H, alkyl, alkenyl, alkynyl, arylalkyl, cycloalkylalkyl, heteroarylalkyl, or heterocyclylalkyl, any of which is substituted or unsubstituted;
each R₂ and R₃ is independently alkylene, alkenylene, or alkynylene, any of which is substituted or unsubstituted;
wherein J is 2-aminoisobutryic acid,
wherein the stitched amino acid sequence binds to glucagon-like peptide 1 receptor (SEQ ID NO:5), and
wherein the stitched amino acid sequence has an alpha helical conformation. In some instances, the pharmaceutically acceptable salt is an acetate, a sulfate, or a chloride.

Also provided herein is a stitched peptide comprising a modified amino acid sequence of the sequence set forth in SEQ ID NO:38 or 31, wherein the peptide comprises a stitch between amino acids corresponding to (a) positions 11, 18, and 25 of SEQ ID NO:38 or 31, (b) positions 12, 19, and 26 of SEQ ID NO:38 or 31, or (c) 6, 13, and 20, and wherein the stitched peptide binds to glucagon-like peptide 1 receptor (SEQ ID NO:5)

Also provided herein is a pharmaceutical composition comprising any one of the foregoing peptides and a pharmaceutically acceptable carrier.

Also provided herein is a pharmaceutical composition comprising: (a) a means for treating diabetes, hyperglycemia, rapid gastric emptying, insulin resistance, cardiovascular disease, Alzheimer’s disease, or Huntington’s disease, and (b) a pharmaceutically acceptable carrier. In some instances, the means for treating diabetes, hyperglycemia, rapid gastric emptying, insulin resistance, cardiovascular disease, Alzheimer’s disease, or Huntington’s disease are stitched GLP-1 peptides.

Also provided herein is a pharmaceutical composition comprising: (a) a means for increasing cAMP levels, and (b) a pharmaceutically acceptable carrier. In some instances, the means for increasing cAMP levels are stitched GLP-1 peptides. In some instances, the cAMP levels are in GLP-1R-expressing cells. In some instances, the cAMP levels are in GLP-1R-expressing cells in a human subject in need thereof.

The disclosure also features a pharmaceutical composition comprising: (a) a means for binding to and agonizing GLP-1 receptor, and (b) a pharmaceutically acceptable carrier. In some instances, the means for binding to GLP-1 receptor are stitched GLP-1 peptides.

Also provided herein is a method of treating diabetes in a human subject in need thereof, the method comprising administering a therapeutically-effective amount of any one of the foregoing peptides to the subject.

Also provided herein is a method of treating hyperglycemia in a human subject in need thereof, the method comprising administering a therapeutically-effective amount of any one of the foregoing peptides to the subject.

Also provided herein is a method of treating rapid gastric emptying, insulin resistance, or cardiovascular disease in a human subject in need thereof, the method comprising administering a therapeutically-effective amount of any one of the foregoing peptides to the subject.

Also provided herein is a method of treating Alzheimer’s disease in a human subject in need thereof, the method comprising administering a therapeutically-effective amount of any one of the foregoing peptides to the subject.

Also provided herein is a method of treating Huntington’s disease in a human subject in need thereof, the method comprising administering a therapeutically-effective amount of any one of the foregoing peptides to the subject.

Also provided herein is a method of increasing cAMP levels in a human subject, the method comprising administering a therapeutically-effective amount of any one of the foregoing peptides to the subject. In some instances, the increase in cAMP levels in the human subject is in GLP-1R-expressing cells in the human subject.

Also provided herein is a method of making a stitched peptide, the method comprising: (a) providing a peptide having the sequence set forth in SEQ ID NO:61, 40, 62, or 33, and (b) cross-linking the peptide. In some instances, the method comprises performing a ring closing metathesis reaction.

Also provided herein is a method of making a stitched peptide, the method comprising: (a) providing a peptide having the sequence set forth in SEQ ID NO: 34, 41, 59-68, 71, 73, 75, 77, 79, 81, 83, and 85, and (b) cross-linking the peptide.

This disclosure also features a method of screening for a stabilized (e.g., stitched) GLP-1 peptide. The method comprises providing a cell expressing a detectable GLP-1R and incubating or exposing the cell to a stabilized (e.g., stitched) peptide. Also included in the method is detecting whether the detectable GLP-1R is internalized, wherein a stabilized (e.g., stitched) peptide that is internalized is selected. In some instances, the cell is a U2OS cell, a CHO cell, a COS cell, a 293 cell, or a Hela cell. In some instances, the GLP-1R is “detectable” by virtue of it being linked, attached, or covalently fused to a detectable label. In certain cases, the detectable label is a fluorescent label. In some cases, the fluorescent label is one of: GFP, YFP, BFP, CFP, EGFP, EYFP, PA-GFP, dsRed, mFruits, mCherry, TagRFP, EosFP, Dronpa, or eqFP611. In one case, the fluorescent label is GFP. In some cases, the selected stabilized (e.g., stitched) peptide is internalized to a greater extent than the unstapled/unstitched GLP-1.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1A: Structures of GLP-1 in complex with GLP-1R demonstrating the burial of the peptide’s N-terminus (PDB: 5NX2) (left) and its helix-in-groove interaction with the extracellular domain of GLP-1R (PDB: 3IOL) (right). X₁X₂GTX₃TSDX₄X₅ is SEQ ID NO: 108, wherein X₁ is N-(2-(1H-imidazol-5-yl)ethyl)-2,2-dimethyl-3-oxobutanyl, X₂ is tetrazolyl-alanine, X₃ is alpha-methyl ortho flurophenylalanine, X₄ is 3-(4′-methoxy-2′-ethyl[1,1′biphenyl]-4-yl, and X₅ is 5-(3,5-dimethylphenyl)-L-norvaline. GTFTSDVSSYLEGQAAKEFIAWLVKG is SEQ ID NO: 109.

FIG. 1B: Amino acid sequences of an i, i+7 staple scanning library of GLP-1, designed to identify optimal location(s) for staple insertion by functional screening. From top to bottom: SEQ ID NOs:6-30 (8 is (R)-α-(7′-octenyl)alanine; X is (S)-α-(4′-pentenyl)alanine).

FIG. 2A-FIG. 2G: GLP-1R internalization assay employed for functional screening of an i, i+7 staple scanning library of GLP-1. FIG. 2A-FIG. 2F are images of fluorescent microscopy of GFP-GLP-1R expressing U2OS cells treated with vehicle (FIG. 2A, FIG. 2C, and FIG. 2E) or GLP-1 peptide (FIG. 2B, FIG. 2D, and FIG. 2F). FIG. 2A and FIG. 2B show hoeschst 33342 staining of nuclei. FIG. 2C and FIG. 2D show GFP-GLP-1R expression. FIG. 2E and FIG. 2F depict a binary mask showing nuclei and vesicles containing internalized GFP-GLP-1R for FIG. 2A and FIG. 2C, and of FIG. 2B and FIG. 2D, respectively. FIG. 2G is a graph showing percentage of cells containing internalized GFP-GLP-1R under each condition (vehicle vs. 0.5 µM GLP-1); data are mean ± s.d. of experiments performed in technical triplicate and repeated three times with similar results.

FIG. 3: (top and middle) Percentage of U2OS cells with internalized GLP-1R in response to treatment with differentially i, i+7-stapled GLP-1 peptides (10, 2.5, or 0.625 µM, data from right to left for each construct). Data are mean ± s.d. for experiments performed in technical quadruplicate and repeated at least twice with similar results. Two biological replicates are shown. (bottom) Helical wheel depiction of GLP-1 (aa 15-35) (SEQ ID NO:110). Residues that engage in direct interactions with GLP-1R are marked by an asterisk.

FIG. 4: Synthesis of i, i+7-stitched peptides by insertion, from C-terminus to N-terminus, S-octenyl alanine (S8; referred to as Z in sequences), bis-pentenyl glycine (Bis-5; referred to as # in sequences), and R-octenyl alanine (R8; referred to as 8 in sequences) at sequential i, i+7 positions within the GLP-1 peptide template.

FIG. 5: Amino acid sequences of double i, i+7-stitched peptides. Ala8 was replaced by Gly (G) or 2-aminoisobutyric acid (Aib, J) to prevent DPP4 proteolysis at this site. From top to bottom: SEQ ID NOs: 31-44 (8 is (R)-α-(7′-octenyl)alanine; # is α,α-Bis(4′-pentenyl)glycine; Z is (S)-α-(7′-octenyl)alanine).

FIG. 6A: Percentage of U2OS cells with internalized GLP-1R in response to treatment with differentially i, i+7-stitched GLP-1 peptides (serial dilution from 5 to 0.15 µM, from right to left). Data are mean ± s.d. for experiments performed in technical quadruplicate and repeated at least twice with similar results. From left to right: SEQ ID NOs:31-44.

FIG. 6B: Percentage of U2OS cells with internalized GLP-1R in response to treatment with differentially i, i+7-stitched GLP-1 peptides (1 µM, 100 nM, 10 nM, from right to left). Data are mean ± s.d. for experiments done in technical quadruplicate and performed at least twice with similar results. An exemplary peptide, SAH-GLP-1(A8G)(16,23,30) is shown to be more active in this lower dose-range than the corresponding unstapled peptide SAH-GLP-1(A8G). From left to right: SEQ ID NOs:31-44.

FIG. 6C: Helix-in-groove depiction of the complex between GLP-1 (amino acids 10-35 of SEQ ID NO:2; helical structure) and the extracellular domain of GLP-1R. Balls and dotted lines are a depiction of the 16,23,30 stitch position, which best preserves the biological activity of GLP-land is localized on the helical surface opposite to the binding interface.

FIG. 6D: Circular dichroism demonstrates that a lead stitched GLP-1 construct (16,23,30), and the corresponding single i, i+7 stapled peptides, maintain the alpha-helical structure of the natural unstapled GLP-1 peptide in solution.

FIG. 6E: Induction of cAMP upon treatment of GLP-1R expressing CHO cells with SAH-GLP-1-A8J(16,23,30), as measured by the cAMP Hunter™ eXpress GLP1R CHO-K1 assay (Eurofins). Based on the cAMP standard curve (EC50: observed, 18 nM; expected, 18.2 nM), the EC50 of SAH-GLP-1-A8J(16,23,30) activity is 160 pM. Data are mean ± s.d. for experiments performed in biological duplicate.

FIG. 7A: SAH-GLP-1(16,23,30), its single-stapled analogs, and wild-type GLP-1 were subjected to proteinase K digestion and intact peptide was quantified over time by LCMS analysis. Data are mean ± s.d. of experiments performed in technical triplicate. t_{½} = 9 minutes for GLP-1, 120 minutes for SAH-GLP-1(16,23), 30 minutes for SAH-GLP-1(23,30), and 220 minutes for SAH-GLP-1(16,23,30).

FIG. 7B: The FDA-approved GLP-1 peptide drug semaglutide demonstrates rapid proteolysis compared to the structurally-stabilized and protease-resistant stapled and stitched GLP-1 analogs shown in FIG. 7A.

FIG. 7C: HXMS of the four-peptide panel performed in triplicate at the indicated deuterium labeling time points. After 10 sec of D₂O exposure, GLP-1 peptides bearing single or stitched i, i + 7 staples showed reduced deuterium exchange by 2.4-3-fold compared to the template peptide, highlighting the conformational rigidity conferred by the staples. Monitoring deuterium exchange over time revealed that i, i + 7 stitching consistently conferred more protection at 3, 25, and 60 min when compared to single i, i +7 stapling alone.

FIG. 8: Glucose tolerance testing was performed after overnight fast by administering the corresponding peptides and vehicle control by IP injection (10 nmol/kg dosing) followed by an IP dose of glucose (2 g/kg) 30 minutes later. Data are mean ± s.d. of serum glucose values measured over time for n=8 mice per treatment condition.

FIG. 9A: Comparative plasma stability of SAH-GLP-1(16,23,30) A8J and semaglutide. Ex vivo mouse plasma stability testing of GLP-1, SAH-GLP-1(16,23,30) A8J, and semaglutide revealed half-lives of 14, 320, and 170 min, respectively. Dotted line, ln(50%).

FIG. 9B and FIG. 9C: Comparative glycemic control by SAH-GLP-1(16,23,30) and semaglutide in a mouse model of diabetes. (FIG. 9B) Diabetic Lepr^{db} mice were treated with a single 10 nmol/kg IP dose of semaglutide, SAH-GLP-1(16,23,30), GLP-1 or vehicle control, followed by serum glucose monitoring over 12 hours. Data are mean ± standard error of mean (s.e.m.) for n=8 mice per treatment condition. (FIG. 9C) The region between 0 and 60 minutes is expanded to better visualize the data at early time points. *, SAH-GLP-1(16,23,30) vs. semaglutide: 15 min, p=0.0032; 30 min, p=0.0001.

FIG. 10A, FIG. 10B, and FIG. 10C: HPLC profiles of i, i+7 staple scanning (FIG. 10A), and double i, i+7 stitched A8G (FIG. 10B) and A8J (FIG. 10C) peptides.

FIG. 11: Nomenclature, sequence compositions, and masses of the synthesized GLP-1 peptides. X is S-pentenyl alanine; 8 is R-octenyl alanine; # is Bis-pentenyl glycine; Z is S-octenyl alanine. From top to bottom: SEQ ID NOs: 4, 6-44,106, and 107.

### DETAILED DESCRIPTION

Glucagon-like peptide 1 (GLP-1) is a natural peptide agonist of the GLP-1 receptor (GLP-1R) found on pancreatic β-cells. Engagement of its receptor stimulates insulin release in a glucose-dependent fashion and increases β-cell mass, two ideal features for pharmacologic management of diabetes. Thus, intensive efforts have focused on developing GLP-1-based peptide agonists of GLP-1R for therapeutic application. A primary challenge has been the naturally short half-life of GLP-1 due to its rapid proteolytic degradation in vivo. This disclosure describes the development of a unique approach to preserving the structure and function of GLP-1 by all-hydrocarbon i, i+7 stitching. The “stitch” is especially well-suited for reinforcing and protecting the particular structure-activity relationship of GLP-1 for GLP-1R interaction. The stitched GLP-1 peptides described herein demonstrated potent biological activity and striking proteolytic stability in vitro and in vivo. This disclosure also features methods for using such stitched peptides alone or in combination with other therapeutic agents in the treatment of type 2 diabetes and/or hyperglycemia. This disclosure also features methods for using such stitched peptides alone or in combination with other therapeutic agents in the treatment of Alzheimer’s disease and Huntington’s disease, or a side-effect or complication thereof. The disclosure also features compositions comprising such stitched peptides and methods of making the stitched peptides. Also provided are methods of screening for stitched peptides for e.g., for use in the methods disclosed herein.

### GLP-1 Peptides

The amino acid sequence of human GLP-1 precursor is provided below (GenBank Accession No. CAA24759):

MKSIYFVAGLFVMLVQGSWQRSLQDTEEKSRSFSASQADPLSDPDQMNED
KRHSQGTFTSDYSKYLDSRRAQDFVQWLMNTKRNRNNIAKRHDEFERHAE
GTFTSDVSSYLEGQAAKEFIAWLVKGRGRDFPEEVAIVEELGRRHADGSF
SDEMNTILDNLAARDFINWLIQTKITDR (SEQ ID NO: 1).

The GLP-1 precursor is processed into an initial peptide product, a 37 amino acid peptide having the sequence

HDEFERHAEGTFTSDVSSYLEGQAAKEFIAWLVK
GRG (SEQ ID NO:2).

This 37 amino acid peptide is susceptible to amidation and proteolytic cleavage, giving rise to two truncated biologically active forms (referred to herein as “GLP-1 peptide”): GLP-1 (7-37) (

HAEGTFTSDVSSYLEGQAAKEFIAWLVKGRG,
SEQ ID NO:3

) and GLP-1 (7-36 amide) (

HAEGTFTSDVSSYLEGQAAKEFIAWLVKGR,
SEQ ID NO:4

). To reduce proteolytic cleavage at Ala8-Glu9 (numbered according to SEQ IDNO:2), Ala8 can be may be substituted with glycine (G) or 2-aminoisobutyric acid (Aib). Exemplary GLP-1 peptides of the disclosure are provided in Table 1, below.

TABLE 1

Exemplary GLP-1 Peptides

| SEQ ID NO | DESCRIPTION | SEQUENCE |
|---|---|---|
| 3 | GLP-1 (7-37) | HAEGTFTSDVSSYLEGQAAKEFIAWLVKGRG |
| 4 | GLP-1 (7-36) | HAEGTFTSDVSSYLEGQAAKEFIAWLVKGR |
| 69 | GLP-1 (7-37) A8G | HGEGTFTSDVSSYLEGQAAKEFIAWLVKGRG |
| 70 | GLP-1 (7-37) A8J | HJEGTFTSDVSSYLEGQAAKEFIAWLVKGRG, wherein J is 2-aminoisobutyric acid |
| 31 | GLP-1 (7-36) A8G | HGEGTFTSDVSSYLEGQAAKEFIAWLVKGR |
| 38 | GLP-1 (7-36) A8J | HJEGTFTSDVSSYLEGQAAKEFIAWLVKGR, wherein J is 2-aminoisobutyric acid |

GLP-1 peptide binds to the GLP-1 receptor (GLP-1R). The GLP-1 peptide residues in the C-terminal portion of GLP-1 (i.e., amino acids 16-37 of SEQ ID NO:2) that engage in direct interactions with GLP-1R (i.e., are on the “GLP-1R-interacting face of the C-terminal portion of GLP-1”) are: Ser18, Glu21, Ala24, Ala25, Lys26, Phe28, Ile29, Leu32, and Val33 (numbered with respect to the positions in SEQ ID NO:2). The rest of the GLP-1 peptide residues in the C-terminal poriton of GLP-1 do not engage in direct interactions with GLP-1R (i.e., are on the “non-GLP-1R-interacting face of the C-terminal portion of GLP-1”). The amino acid sequence of mature human GLP-1R is provided below (amino acids 24-463 of GenBank Accession No. NP_002053.3):

RPQGATVSLWETVQKWREYRRQCQRSLTEDPPPATDLFCNRTFDEYACWP
DGEPGSFVNVSCPWYLPWASSVPQGHVYRFCTAEGLWLQKDNSSLPWRDL
SECEESKRGERSSPEEQLLFLYIIYTVGYALSFSALVIASAILLGFRHLH
CTRNYIHLNLFASFILRALSVFIKDAALKWMYSTAAQQHQWDGLLSYQDS
LSCRLVFLLMQYCVAANYYWLLVEGVYLYTLLAFSVLSEQWIFRLYVSIG
WGVPLLFVVPWGIVKYLYEDEGCWTRNSNMNYWLIIRLPILFAIGVNFLI
FVRVICIVVSKLKANLMCKTDIKCRLAKSTLTLIPLLGTHEVIFAFVMDE
HARGTLRFIKLFTELSFTSFQGLMVAILYCFVNNEVQLEFRKSWERWRLE
HLHIQRDSSMKPLKCPTSSLSSGATAGSSMYTATCQASCS
(SEQ ID NO:5).

Provided herein are peptides comprising a modified amino acid sequence of a GLP-1 peptide described herein. The peptides are modified to introduce structural stabilization to the peptide (e.g., to maintain alpha-helicity of the peptide). The structural stabilization is by “stitching” the peptide. In some cases, the stitch is a hydrocarbon stitch. The modifications to introduce structural stabilization (e.g., internal cross-linking, e.g., stitching) into the GLP-1 peptides described herein are positioned at: (i) the amino acid positions in the GLP-1 peptide corresponding to residues 16, 23, and 30 of the amino acid sequence set forth in SEQ ID NO:2; (ii) the amino acid positions in the GLP-1 peptide corresponding to residues 17, 24, and 31 of the amino acid sequence set forth in SEQ ID NO:2; (iii) the amino acid positions in the GLP-1 peptide corresponding to residues 18, 25, and 32 of the amino acid sequence set forth in SEQ ID NO:2; or (iv) the amino acid positions in the GLP-1 peptide corresponding to residues 12, 19, and 26 of the amino acid sequence set forth in SEQ ID NO:2. In some instances, the stitch is located at the amino acid positions in the GLP-1 peptide corresponding to residues 16, 23, and 30 of the amino acid sequence set forth in SEQ ID NO:2. In some instances, the stitch is located at the amino acid positions in the GLP-1 peptide corresponding to residues 17, 24, and 31 of the amino acid sequence set forth in SEQ ID NO:2. In some instances, the stitch is located at the amino acid positions in the GLP-1 peptide corresponding to residues 18, 25, and 32 of the amino acid sequence set forth in SEQ ID NO:2. In some instances, the stitch is located at the amino acid positions in the GLP-1 peptide corresponding to residues 12, 19, and 26 of the amino acid sequence set forth in SEQ ID NO:2. In certain instances, the structurally-stabilized (e.g., internally cross-linked, e.g., stitched) GLP-1 peptides described herein may also contain one or more (e.g., 1, 2, 3, 4, 5, 6, 7) additional amino acid substitutions (relative to the wild type GLP-1 peptide sequence), e.g., one or more (e.g., 1, 2, 3, 4, 5, 6, 7) conservative and/or non-conservative amino acid substitutions (i.e., one or more amino acid substitutions in addition to the amino acid substitutions made to the GLP-1 to impart the structural stabilization). In certain instances, the structurally-stabilized (e.g., internally cross-linked, e.g., stitched) GLP-1 peptide comprises a glycine at the amino acid position corresponding to position 8 of the amino acid sequence set forth in SEQ ID NO:2. In certain instances, the structurally-stabilized (e.g., internally cross-linked, e.g., stitched) GLP-1 peptide comprises a 2-aminoisobutyric acid at the amino acid position corresponding to position 8 of the amino acid sequence set forth in SEQ ID NO:2. In certain instances, these additional substitution(s) are of amino acids in the C-terminal portion of GLP-1 that directly interact with GLP-1R (e.g., one or more of Ser18, Glu21, Ala24, Ala25, Lys26, Phe28, Ile29, Leu32, and Val33 (numbered with respect to the positions in SEQ ID NO:2)). In certain instances, these additional substitution(s) are of amino acids in the C-terminal portion of GLP-1 that do not engage in direct interaction with GLP-1R. In certain instances, these additional substitutions are of both amino acids in the C-terminal portion of GLP-1 that directly interact with GLP-1R and amino acids in the C-terminal portion of GLP-1 that do not engage in direct interaction with GLP-1R. In certain instances, the structurally-stabilized (e.g., internally cross-linked, e.g., stitched) GLP-1 peptides described herein may also contain one or more (e.g., 1, 2, 3, 4, 5, 6, 7, 8, 9, 10) deletions from the N- and/or C-terminus of the GLP-1 peptide. For example, the structurally-stabilized (e.g., internally cross-linked, e.g., stitched) GLP-1 peptides may be at least 15 amino acids in length (to accommodate the stitches at (i) the amino acid positions corresponding to residues 16, 23, and 30 of the amino acid sequence set forth in SEQ ID NO:2, (ii) the amino acid positions corresponding to residues 17, 24, and 31 of the amino acid sequence set forth in SEQ ID NO:2, (iii) the amino acid positions in the GLP-1 peptide corresponding to residues 18, 25, and 32 of the amino acid sequence set forth in SEQ ID NO:2; or (iv) the amino acid positions in the GLP-1 peptide corresponding to residues 12, 19, and 26 of the amino acid sequence set forth in SEQ ID NO:2) but less than 30 (e.g., 29, 28, 27, 26, 25, 24, 23, 22, 21, 20, 19, 18, 17, 16, 15) amino acids in length. In certain instances, the structurally-stabilized (e.g., internally cross-linked, e.g., stitched) GLP-1 peptides are 15-50 amino acids in length. In certain instances, the structurally-stabilized (e.g., internally cross-linked, e.g., stitched) GLP-1 peptides are 15-40 amino acids in length. In certain instances, the structurally-stabilized (e.g., internally cross-linked, e.g., stitched) GLP-1 peptides are 15-31 amino acids in length. In certain instances, the structurally-stabilized (e.g., internally cross-linked, e.g., stitched) GLP-1 peptides are 15-30 amino acids in length. In certain instances, the structurally-stabilized (e.g., internally cross-linked, e.g., stitched) GLP-1 peptides are 15-25 amino acids in length. In certain instances, the structurally-stabilized (e.g., internally cross-linked, e.g., stitched) GLP-1 peptides are 15-20 amino acids in length. In certain instances, the structurally-stabilized (e.g., internally cross-linked, e.g., stitched) GLP-1 peptides are 30-50 amino acids in length. In certain instances, the structurally-stabilized (e.g., internally cross-linked, e.g., stitched) GLP-1 peptides are 30-40 amino acids in length. In certain instances, the structurally-stabilized (e.g., internally cross-linked, e.g., stitched) GLP-1 peptides are 31 amino acids in length. In certain instances, the structurally-stabilized (e.g., internally cross-linked, e.g., stitched) GLP-1 peptides are 30 amino acids in length.

In certain instances, the GLP-1 peptides of this disclosure can have 1, 2, 3, 4, or 5 amino acid substitutions in the amino acid sequence of any one of SEQ ID NOs: 3, 4, 31, 38, 69, and 70 (e.g., 1, 2, 3, 4, or 5 amino acids are conservatively or nonconservatively substituted). For example, in certain instances, the GLP-1 peptide of this disclosure comprises a modified amino acid sequence of the sequence set forth in SEQ ID NO:38, wherein the modified amino acid sequence comprises the amino acid sequence set forth in SEQ ID NO:38 having 1, 2, 3, 4, or 5 amino acid substitutions in the SEQ ID NO:38 sequence (e.g., the modified amino acid sequence comprises the amino acid sequence set forth in SEQ ID NO:38, except that 1, 2, 3, 4, or 5 amino acids of the amino acid sequence set forth in SEQ ID NO:38 are conservatively or nonconservatively substituted). In another example, in certain instances, the GLP-1 peptide of this disclosure comprises a modified amino acid sequence of the sequence set forth in SEQ ID NO:31, wherein the modified amino acid sequence comprises the amino acid sequence set forth in SEQ ID NO:31 having 1, 2, 3, 4, or 5 amino acid substitutions in the SEQ ID NO:31 sequence (e.g., the modified amino acid sequence comprises the amino acid sequence set forth in SEQ ID NO:31, except that 1, 2, 3, 4, or 5 amino acids of the amino acid sequence set forth in SEQ ID NO:31 are conservatively or nonconservatively substituted). In another example, in certain instances, the GLP-1 peptide of this disclosure comprises a modified amino acid sequence of the sequence set forth in SEQ ID NO:69, wherein the modified amino acid sequence comprises the amino acid sequence set forth in SEQ ID NO:69 having 1, 2, 3, 4, or 5 amino acid substitutions in the SEQ ID NO:69 sequence (e.g., the modified amino acid sequence comprises the amino acid sequence set forth in SEQ ID NO:69, except that 1, 2, 3, 4, or 5 amino acids of the amino acid sequence set forth in SEQ ID NO:69 are conservatively or nonconservatively substituted). In another example, in certain instances, the GLP-1 peptide of this disclosure comprises a modified amino acid sequence of the sequence set forth in SEQ ID NO:70, wherein the modified amino acid sequence comprises the amino acid sequence set forth in SEQ ID NO:70 having 1, 2, 3, 4, or 5 amino acid substitutions in the SEQ ID NO:70 sequence (e.g., the modified amino acid sequence comprises the amino acid sequence set forth in SEQ ID NO:70, except that 1, 2, 3, 4, or 5 amino acids of the amino acid sequence set forth in SEQ ID NO:70 are conservatively or nonconservatively substituted). A “conservative amino acid substitution” means that the substitution replaces one amino acid with another amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The amino acid substitutions in the amino acid sequence set forth in any one of SEQ ID NOs: 3, 4, 31, 38, 69, and 70 can be of amino acids that directly interact or do not directly interact with GLP-1R. Examples of amino acids in the C-terminal portion of GLP-1 that directly interact with GLP-1R are Ser18, Glu21, Ala24, Ala25, Lys26, Phe28, Ile29, Leu32, and Val33 (numbered with respect to the positions in SEQ ID NO:2). Much greater variability is permitted in the GLP-1R amino acids in the C-terminal portion of GLP-1 that do not directly interact with GLP-1R than in the amino acids in the C-terminal portion of GLP-1 that directly interact with GLP-1. In fact, just about every one of the amino acids in the C-terminal portion of GLP-1 that do not directly interact with GLP-1 can be substituted (e.g., conservative or non-conservative amino acid substitutions or substitution with alanine). In certain instances, 1, 2, or 3 GLP-1 amino acids in the C-terminal portion of GLP-1 that directly interact with GLP-1R are substituted with another amino acid. In some instances, the substitution(s) is/are a conservative amino acid substitution. In other instances, the substitution(s) is/are a non-conservative amino acid substitution. In some instances, where there are more than one amino acid substitutions, the substitutions are both conservative and non-conservative amino acid substitutions. In some instances, where there are more than one amino acid substitutions, each of the substitutions are conservative amino acid substitutions. In some cases, where one to three amino acids (e.g., 1, 2, or 3) of the amino acid sequence of any one of SEQ ID NOs: 3, 4, 31, 38, 69, and 70 are substituted, the substitutions are all of the GLP-1 peptide residues in the C-terminal portion of GLP-1 that do not directly interact with GLP-1R, so long as the modified GLP-1 peptide retains the ability to interact with GLP-1R. In some cases, where one to three amino acids (e.g., 1, 2, or 3) of the amino acid sequence of any one of SEQ ID NOs: 3, 4, 31, 38, 69, and 70 are substituted, the substitutions are all of GLP-1 peptide amino acids in the C-terminal portion of GLP-1 that directly interact with GLP-1R, so long as the modified GLP-1 peptide retains the ability to interact with GLP-1R. In some cases, where one to three amino acids (e.g., 1, 2, or 3) of the amino acid sequence of any one of SEQ ID NOs:3, 4, 31, 38, 69, and 70 are substituted, the substitutions are of both GLP-1 peptide amino acids in the C-terminal portion of GLP-1 that directly interact with GLP-1R and GLP-1 peptide amino acids in the C-terminal portion of GLP-1 that do not directly interact with GLP-1R. In certain instances, the substituted amino acid(s) are selected from the group consisting of L-Ala, D-Ala, Aib, Sar, Ser, a substituted alanine, or a substituted glycine derivative.

In certain instances, the GLP-1 peptides of this disclosure can have 1, 2, or 3 amino acids removed/deleted from the C-terminus of the sequence set forth in any one of SEQ ID NOs: 3, 4, 31, 38, 69, and 70. In certain instances, the GLP-1 peptides of this disclosure can have 1, 2, 3, 4, or 5, amino acids removed/deleted from the N-terminus of the sequence set forth in any one of SEQ ID NOs: 3, 4, 31, 38, 69, and 70. In certain instances, these removed amino acids can be replaced with 1-6 (e.g., 1, 2, 3, 4, 5, or 6) amino acids selected from the group consisting of L-Ala, D-Ala, Aib, Sar, Ser, a substituted alanine, or a substituted glycine derivative.

The disclosure also encompasses GLP-1 peptides that are at least 14% (e.g., at least 14% to 50%, at least 14% to 45%, at least 14% to 40%, at least 14% to 35%, at least 14% to 30%, at least 14% to 25%, at least 14% to 20%, at least 20% to 50%, at least 20% to 45%, at least 20% to 40%, at least 20% to 35%, at least 20% to 30%, at least 20% to 25%, at least 15%, at least 20%, at least 27%, at least 34%, at least 40% at least 45%, at least 50%, at least 55%, at least 60%, at least 65%, at least 70%, at least 75%, at least 80%, at least 85%, at least 90%, at least 95%, at least 98%, at least 99%, or 100%) identical to the amino acid sequence of any one of SEQ ID NOs: 3, 4, 31, 38, 69, and 70. The variability in amino acid sequence of any one of SEQ ID NOs: 3, 4, 31, 38, 69, and 70 can be in the N-terminal portion (i.e., amino acids 6-15 of SEQ ID NO:2) of GLP-1, on the GLP-1R-interacting face of the C-terminal portion (i.e., amino acids 16-37 of SEQ ID NO:2) of GLP-1, and/or on the GLP-1R-non-interacting face of the C-terminal portion of GLP-1. Just about every one of the GLP-1 peptide C-terminal amino acids that do not directly interact with GLP-1R can be varied. The GLP-1 peptide amino acids that directly interact with GLP-1R can also be varied. Examples of GLP-1 amino acids in the C-terminal portion of GLP-1 that directly interact with GLP-1R are Ser18, Glu21, Ala24, Ala25, Lys26, Phe28, Ile29, Leu32, and Val33 (numbered with respect to the positions in SEQ ID NO:2). In specific instances, the GLP-1 peptide comprises an amino acid sequence that is at least 90% identical to the amino acid sequence of any one of SEQ ID NOs: 3, 4, 31, 38, 69, and 70. In specific instances, the GLP-1 peptide comprises an amino acid sequence that is at least 95%, at least 96%, at least 97%, at least 98%, at least 99% identical to the amino acid sequence of any one of SEQ ID NOs: 3, 4, 31, 38, 69, and 70. In specific instances, the GLP-1 peptide comprises the amino acid sequence of any one of SEQ ID NOs: 3, 4, 31, 38, 69, and 70. In specific instances, the GLP-1 peptide consists of the amino acid sequence of any one of SEQ ID NOs: 3, 4, 31, 38, 69, and 70. Methods for determining percent identity between amino acid sequences are known in the art. For example, the sequences are aligned for optimal comparison purposes (e.g., gaps can be introduced in one or both of a first and a second amino acid or nucleic acid sequence for optimal alignment and nonhomologous sequences can be disregarded for comparison purposes). In a preferred instance, the length of a reference sequence aligned for comparison purposes is at least 30%, preferably at least 40%, more preferably at least 50%, even more preferably at least 60%, and even more preferably at least 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The determination of percent identity between two amino acid sequences is accomplished using the BLAST 2.0 program. Sequence comparison is performed using an ungapped alignment and using the default parameters (Blossom 62 matrix, gap existence cost of 11, per residue gapped cost of 1, and a lambda ratio of 0.85). The mathematical algorithm used in BLAST programs is described in Altschul et al. (Nucleic Acids Res. 25:3389-3402, 1997).

In some instances, the disclosure features variants of the amino acid sequence of any one of SEQ ID NOs: 3, 4, 31, 38, 69, and 70, wherein the peptide variants noncovalently bind to GLP-1R.

This disclosure also features structurally-stabilized versions (e.g., internally cross-linked, e.g., stitched) of these GLP-1 peptides. Three or more residues of the GLP-1 peptides described herein separated by 6 amino acids (i.e., residues i, i+7, and i+14), are replaced with non-natural amino acids that can form a cross-link by olefin methathesis. The cross-link is positioned in these variants at locations that do not disrupt binding of the GLP-1 peptide to GLP-1R. In some instances, the GLP-1 peptides are structurally-stabilized by a hydrocarbon stitch, a lactam stitch; a UV-cycloaddition stitch; an oxime stitch; a thioether stitch; a double-click stitch; a bis-lactam stitch; a bis-arylation stitch; or a combination of any two or more thereof. In some instances, the GLP-1 peptides are structurally-stabilized by a hydrocarbon stitch.

The GLP-1 peptides described herein can be optimized for therapeutic use. For example, if any of the above-described GLP-1 peptides cause membrane disruption (cell lysis), the peptides can be optimized by lowering the overall peptide hydrophobicity. This can for example be achieved by substituting especially hydrophobic residues with an amino acid with lower hydrophobicity (e.g., alanine). Membrane disruption can also be lowered by reducing the overall positive charge of the peptide. This can be accomplished by substituting basic residues with uncharged or acidic residues. In certain instances, both the overall peptide hydrophobicity and the overall positive charge of the peptide are lowered.

### Stitched Peptides

A peptide helix is an important mediator of key protein-protein interactions that regulate many important biological processes; however, when such a helix is taken out of its context within a protein and prepared in isolation, it usually adopts a random coil conformation, leading to a drastic reduction in biological activity and thus diminished therapeutic potential. The present disclosure provides stitched GLP-1 peptides. The term “peptide stitching,” as used herein, refers to multiple and tandem “stapling” events in a single polypeptide chain to provide a “stitched” (e.g., tandem or multiply stapled) polypeptide, in which two staples, for example, are linked to a common residue. “Peptide stapling” is a term coined from a synthetic methodology wherein two olefin-containing side-chains (e.g., cross-linkable side chains) present in a polypeptide chain are covalently joined (e.g., “stapled together”) using a ring-closing metathesis (RCM) reaction to form a cross-linked ring (see, e.g., Blackwell et al., J. Org. Chem., 66: 5291-5302, 2001; Angew et al., Chem. Int. Ed. 37:3281, 1994). As used herein, the term “peptide stapling” includes the joining of two (e.g., at least one pair of) double bond-containing side-chains, triple bond-containing side-chains, or double bond-containing and triple bond-containing side chains, which may be present in a polypeptide chain, using any number of reaction conditions and/or catalysts to facilitate such a reaction, to provide a singly “stapled” polypeptide. Peptide stitching is disclosed, e.g., in WO 2008/121767 and WO 2010/068684, which are both hereby incorporated by reference in their entirety. In some instances, staples, as used herein, can retain the unsaturated bond or can be reduced.

The present disclosure includes stitched GLP-1 peptides (such as those described above) comprising three modified amino acids joined by two internal (intramolecular) cross-links, thereby forming a “stitch”. See, e.g., Balaram P. Cur. Opin. Struct. Biol. 1992;2:845; Kemp DS, et al., J. Am. Chem. Soc. 1996;118:4240; Orner BP, et al., J. Am. Chem. Soc. 2001;123:5382; Chin JW, et al., Int. Ed. 2001;40:3806; Chapman RN, et al., J. Am. Chem. Soc. 2004; 126:12252; Home WS, et al., Chem., Int. Ed. 2008;47:2853; Madden et al., Chem Commun (Camb). 2009 Oct 7; (37): 5588-5590; Lau et al., Chem. Soc. Rev., 2015,44:91-102; and Gunnoo et al., Org. Biomol. Chem., 2016,14:8002-8013; all of which are incorporated by reference herein in its entirety, for examples of stapling and stitching mechanisms.

In certain instances, one or more of the GLP-1 peptides described herein can be structurally-stabilized by peptide stitching. A peptide is “structurally-stabilized” in that it maintains its native secondary structure. For example, stitching allows a peptide, predisposed to have an α-helical secondary structure, to maintain its native α-helical conformation. This secondary structure increases resistance of the peptide to proteolytic cleavage and heat, and also may increase target binding affinity, hydrophobicity, and cell permeability. Accordingly, the stitched (cross-linked) peptides described herein have improved biological activity relative to a corresponding non-stitched (un-cross-linked) polypeptide.

In some instances, the GLP-1 peptides of this disclosure are structurally-stabilized by a hydrocarbon stitch, a lactam stitch; a UV-cycloaddition stitch; an oxime stitch; a thioether stitch; a double-click stitch; a bis-lactam stitch; a bis-arylation stitch; or a combination of any two or more thereof. In one instance, the GLP-1 peptides of this disclosure are structurally-stabilized by a hydrocarbon stitch. In some instances, the stitched peptide is a cross-linked version of a polypeptide comprising or consisting of any one of the amino acids sequences of SEQ ID NOs: 3, 4, 31, 38, 69, and 70. In some instances, the stitched peptide is a hydrocarbon stitched version of a polypeptide comprising or consisting of the amino acid sequence of any one of SEQ ID NOs: 3, 4, 31, 38, 69, and 70. In some instances, the stitched peptide is a peptide comprising or consisting of the amino acid sequence of any one of SEQ ID NOs: 3, 4, 31, 38, 69, and 70, except that three amino acids of the amino acid sequence of any one of SEQ ID NOs: 3, 4, 31, 38, 69, and 70, respectively, are each replaced with a non-natural amino acid capable of forming a stitch (i.e., “stitching amino acids”, e.g., non-natural amino acids with olefinic side chains, e.g., S5 (i.e., S-pentenyl alanine), R8 (i.e., R-octenyl alanine), bis-5 (i.e., bis-pentenyl glycine)). The three amino acids capable of forming a stitch (i.e., “stitching amino acids”) are separated by six amino acids (between the first and second amino acids of the stitch) or by 13 amino acids (between the first and third amino acids of the stitch) (i.e., are at positions i, i+7, and i+14, i.e., an “i+7 stitch”). In certain instances, the stitched peptide includes at least three (e.g., 3, 4, 5, 6) amino acid substitutions, wherein the substituted amino acids are separated by six amino acids, and wherein the substituted amino acids are non-natural amino acids with olefinic side chains. There are many known non-natural or unnatural amino acids that may be used as stitching amino acids, any of which may be included in the peptides of the present disclosure. Some examples of stitching amino acids are R-octenyl alanine (“R8”, e.g., (R)-α-(7′-octenyl)alanine), S-octenyl alanine (“S8”, e.g., (S)-α-(7′-octenyl)alanine), bis-pentenyl glycine (“bis-5”, e.g., α,α-Bis(4′-pentenyl)glycine), S-pentenyl alanine (“S5”, e.g., (S)-α-(4′-pentenyl)alanine), R-pentenyl alanine (“R5”, e.g., (R)-α-(4′-pentenyl)alanine), Bis-octenyl glycine (“bis-8”, e.g., α,α-Bis(7′-octenyl)glycine), 4-hydroxyproline, desmosine, gamma-aminobutyric acid, beta-cyanoalanine, norvaline, 4-(E)-butenyl-4(R)-methyl-N- methyl-L-threonine, N-methyl-L-leucine, 1-amino-cyclopropanecarboxylic acid, 1- amino-2-phenyl-cyclopropanecarboxylic acid, 1-amino-cyclobutanecarboxylic acid, 4- amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3- diaminopropionic acid, 2,4-diaminobutyric acid, 2-aminoheptanedioic acid, 4- (aminomethyl)benzoic acid, 4-aminobenzoic acid, ortho-, meta- and /para-substituted phenylalanines (e.g., substituted with -C(=O)C₆H₅; -CF₃; -CN; -halo; -NO₂; CH₃), disubstituted phenylalanines, substituted tyrosines (e.g., further substituted with -C=O)C₆H₅; -CF₃; -CN; -halo; -NO₂; CH₃), and statine. Additionally, amino acids can be derivatized to include amino acid residues that are hydroxylated, phosphorylated, sulfonated, acylated, or glycosylated.

In some instances, the amino acids forming the stitch (also referred to as the “stitching amino acids”) are R8, bis-5, and S8. In some instances, the amino acids forming the stitch (also referred to as the “stitching amino acids”) are (R)-α-(7′-octenyl)alanine), α,α-Bis(4′-pentenyl)glycine, and (S)-α-(7′-octenyl)alanine. In some instances, the amino acids forming the stitch (also referred to as the “stitching amino acids”) are R8, bis-5, and R8, at positions i, i+7, and i+14, respectively. In some instances, the amino acids forming the stitch (also referred to as the “stitching amino acids”) are (R)-α-(7′-octenyl)alanine), α,α-Bis(4′-pentenyl)glycine, and (R)-α-(7′-octenyl)alanine.

In some instances, the amino acids forming the stitch (also referred to as the “stitching amino acids”) are S8, bis-5, and R8, at positions i, i+7, and i+14, respectively. In some instances, the amino acids forming the stitch (also referred to as the “stitching amino acids”) are (S)-α-(7′-octenyl)alanine), α,α-Bis(4′-pentenyl)glycine, and (R)-α-(7′-octenyl)alanine, at positions i, i+7, and i+14, respectively. In some instances, the amino acids forming the stitch are S8, bis-5, and S8, at positions i, i+7, and i+14, respectively. In some instances, the amino acids forming the stitch (also referred to as the “stitching amino acids”) are (S)-α-(7′-octenyl)alanine), α,α-Bis(4′-pentenyl)glycine, and (S)-α-(7′-octenyl)alanine, at positions i, i+7, and i+14, respectively.

In some instances, the amino acids forming the stitch (also referred to as the “stitching amino acids”) are R5, bis-8, and S5, at positions i, i+7, and i+14, respectively. In some instances, the amino acids forming the stitch (also referred to as the “stitching amino acids”) are (R)-α-(4′-pentenyl)alanine), α,α-Bis(7′-octenyl)glycine, and (S)-α-(4′-pentenyl)alanine, at positions i, i+7, and i+14, respectively. In some instances, the amino acids forming the stitch (also referred to as the “stitching amino acids”) are R5, bis-8, and R5, at positions i, i+7, and i+14, respectively. In some instances, the amino acids forming the stitch (also referred to as the “stitching amino acids”) are (R)-α-(4′-pentenyl)alanine), α,α-Bis(7′-octenyl)glycine, and (R)-α-(4′-pentenyl)alanine, at positions i, i+7, and i+14, respectively.

In some instances, the amino acids forming the stitch (also referred to as the “stitching amino acids”) are S5, bis-8, and R5, at positions i, i+7, and i+14, respectively. In some instances, the amino acids forming the stitch (also referred to as the “stitching amino acids”) are (S)-α-(4′-pentenyl)alanine), α,α-Bis(7′-octenyl)glycine, and (R)-α-(4′-pentenyl)alanine, at positions i, i+7, and i+14, respectively. In some instances, the amino acids forming the stitch are S5, bis-8, and S5, at positions i, i+7, and i+14, respectively. In some instances, the amino acids forming the stitch (also referred to as the “stitching amino acids”) are (S)-α-(4′-pentenyl)alanine), α,α-Bis(7′-octenyl)glycine, and (S)-α-(4′-pentenyl)alanine, at positions i, i+7, and i+14, respectively.

Hydrocarbon stitched peptides include at least two tethers (linkages) between three non-natural amino acids (e.g., non-natural amino acids with olefinic side chains, e.g., S8, R8, and bis-5), which tethers significantly enhance the α-helical secondary structure and stability of the peptide. Generally, the tether extends across the length of one or two helical turns (i.e., about 3.4 or about 7 amino acids). Accordingly, amino acids positioned at i, i+7, and i+14 are ideal candidates for chemical modification and cross-linking (forming an “i+7 stitch”). Thus, for example, where a peptide has the sequence ... X1, X2, X3, X4, X5, X6, X7, X8, X9, X10, X11, X12, X13, X14, X15 ... , cross-links between X1 and X7 and between X7 and X14 are useful hydrocarbon stitched forms of that peptide, as are cross-links between X2 and X8 and between X8 and X15, etc. The use of multiple cross-links is very effective at stabilizing and optimizing the peptide, especially with increasing peptide length. Additional description regarding making and use of hydrocarbon stapled polypeptides can be found, e.g., in U.S. Pat. Publication Nos. 2012/0172285, 2010/0286057, and 2005/0250680, the contents of all of which are incorporated by reference herein in their entireties.

In a peptide to be stitched, amino acids that interfere with (e.g., inhibit or reduce the efficiency of) the stitching reaction should be substituted with amino acids that do not interfere with (e.g., do not inhibit or do not substantially reduce the efficiency of) the stapling reaction.

In some instances, the stitch is located at the amino acid positions in a GLP-1 peptide corresponding to positions 16, 23, and 30 of the amino acid sequence set forth in SEQ ID NO:2. In some instances in which the GLP-1 peptide comprises or consists of the amino acid sequence of SEQ ID NO:38, the stitch is located at the amino acid positions in the GLP-1 peptide corresponding to positions 16, 23, and 30 of the amino acid sequence set forth in SEQ ID NO:2 (i.e., positions 10, 17, and 24 of the amino acid sequence set forth in SEQ ID NO:38). In some instances in which the GLP-1 peptide comprises or consists of the amino acid sequence of SEQ ID NO:31, the stitch is located at the amino acid positions in the GLP-1 peptide corresponding to positions 16, 23, and 30 of the amino acid sequence set forth in SEQ ID NO:2 (i.e., positions 10, 17, and 24 of the amino acid sequence set forth in SEQ ID NO:31). In some instances in which the GLP-1 peptide comprises or consists of the amino acid sequence of SEQ ID NO:69, the stitch is located at the amino acid positions in the GLP-1 peptide corresponding to positions 16, 23, and 30 of the amino acid sequence set forth in SEQ ID NO:2 (i.e., positions 10, 17, and 24 of the amino acid sequence set forth in SEQ ID NO:69). In some instances in which the GLP-1 peptide comprises or consists of the amino acid sequence of SEQ ID NO:70, the stitch is located at the amino acid positions in the GLP-1 peptide corresponding to positions 16, 23, and 30 of the amino acid sequence set forth in SEQ ID NO:2 (i.e., positions 10, 17, and 24 of the amino acid sequence set forth in SEQ ID NO:70).

In some instances, the stitch is located at the amino acid positions in the GLP-1 peptide corresponding to positions 17, 24, and 31 of the amino acid sequence set forth in SEQ ID NO:2. In some instances in which the GLP-1 peptide comprises or consists of the amino acid sequence of SEQ ID NO:38, the stitch is located at the amino acid positions in the GLP-1 peptide corresponding to positions 17, 24, and 31 of the amino acid sequence set forth in SEQ ID NO:2 (i.e., positions 11, 18, and 25 of the amino acid sequence set forth in SEQ ID NO:38). In some instances in which the GLP-1 peptide comprises or consists of the amino acid sequence of SEQ ID NO:31, the stitch is located at the amino acid positions in the GLP-1 peptide corresponding to positions 17, 24, and 31 of the amino acid sequence set forth in SEQ ID NO:2 (i.e., positions 11, 18, and 25 of the amino acid sequence set forth in SEQ ID NO:31). In some instances in which the GLP-1 peptide comprises or consists of the amino acid sequence of SEQ ID NO:69, the stitch is located at the amino acid positions in the GLP-1 peptide corresponding to positions 17, 24, and 31 of the amino acid sequence set forth in SEQ ID NO:2 (i.e., positions 11, 18, and 25 of the amino acid sequence set forth in SEQ ID NO:69). In some instances in which the GLP-1 peptide comprises or consists of the amino acid sequence of SEQ ID NO:70, the stitch is located at the amino acid positions in the GLP-1 peptide corresponding to positions 17, 24, and 31 of the amino acid sequence set forth in SEQ ID NO:2 (i.e., positions 11, 18, and 25 of the amino acid sequence set forth in SEQ ID NO:70).

In some instances, the stitch is located at the amino acid positions in a GLP-1 peptide corresponding to positions 18, 25, and 32 of the amino acid sequence set forth in SEQ ID NO:2. In some instances in which the GLP-1 peptide comprises or consists of the amino acid sequence of SEQ ID NO:38 , the stitch is located at the amino acid positions in the GLP-1 peptide corresponding to positions 18, 25, and 32 of the amino acid sequence set forth in SEQ ID NO:2 (i.e., positions 12, 19, and 26 of the amino acid sequence set forth in SEQ ID NO:38). In some instances in which the GLP-1 peptide comprises or consists of the amino acid sequence of SEQ ID NO:31 , the stitch is located at the amino acid positions in the GLP-1 peptide corresponding to positions 18, 25, and 32 of the amino acid sequence set forth in SEQ ID NO:2 (i.e., positions 12, 19, and 26 of the amino acid sequence set forth in SEQ ID NO:31). In some instances in which the GLP-1 peptide comprises or consists of the amino acid sequence of SEQ ID NO:69 , the stitch is located at the amino acid positions in the GLP-1 peptide corresponding to positions 18, 25, and 32 of the amino acid sequence set forth in SEQ ID NO:2 (i.e., positions 12, 19, and 26 of the amino acid sequence set forth in SEQ ID NO:69). In some instances in which the GLP-1 peptide comprises or consists of the amino acid sequence of SEQ ID NO:70, the stitch is located at the amino acid positions in the GLP-1 peptide corresponding to positions 18, 25, and 32 of the amino acid sequence set forth in SEQ ID NO:2 (i.e., positions 12, 19, and 26 of the amino acid sequence set forth in SEQ ID NO:70).

In some instances, the stitch is located at the amino acid positions in a GLP-1 peptide corresponding to positions 12, 19, and 26 of the amino acid sequence set forth in SEQ ID NO:2. In some instances in which the GLP-1 peptide comprises or consists of the amino acid sequence of SEQ ID NO:38, the stitch is located at the amino acid positions in the GLP-1 peptide corresponding to positions 12, 19, and 26 of the amino acid sequence set forth in SEQ ID NO:2 (i.e., positions 6, 13, and 20 of the amino acid sequence set forth in SEQ ID NO:38). In some instances in which the GLP-1 peptide comprises or consists of the amino acid sequence of SEQ ID NO:31 , the stitch is located at the amino acid positions in the GLP-1 peptide corresponding to positions 12, 19, and 26 of the amino acid sequence set forth in SEQ ID NO:2 (i.e., positions 6, 13, and 20 of the amino acid sequence set forth in SEQ ID NO:31). In some instances in which the GLP-1 peptide comprises or consists of the amino acid sequence of SEQ ID NO:69 , the stitch is located at the amino acid positions in the GLP-1 peptide corresponding to positions 12, 19, and 26 of the amino acid sequence set forth in SEQ ID NO:2 (i.e., positions 6, 13, and 20 of the amino acid sequence set forth in SEQ ID NO:69). In some instances in which the GLP-1 peptide comprises or consists of the amino acid sequence of SEQ ID NO:70 , the stitch is located at the amino acid positions in the GLP-1 peptide corresponding to positions 12, 19, and 26 of the amino acid sequence set forth in SEQ ID NO:2 (i.e., positions 6, 13, and 20 of the amino acid sequence set forth in SEQ ID NO:70).

In some instances, the stitched peptide comprises the amino acid sequence of SEQ ID NO:38, wherein each of positions 10, 17, and 24 of the amino acid sequence set forth in SEQ ID NO:38 is replaced with a stapling amino acid, wherein a sidechain of the stapling amino acid at position 10 is cross-linked to a sidechain of the stapling amino acid at position 17 and a sidechain of the stapling amino acid at position 17 is cross-linked to a side chain of the stapling amino acid at position 24, and wherein the peptide binds to glucagon-like peptide 1 receptor (SEQ ID NO:5). In some instances, the stitched peptide comprises the amino acid sequence of SEQ ID NO:31, wherein each of positions 10, 17, and 24 of the amino acid sequence set forth in SEQ ID NO:31 is replaced with a stapling amino acid, wherein a sidechain of the stapling amino acid at position 10 is cross-linked to a sidechain of the stapling amino acid at position 17 and a sidechain of the stapling amino acid at position 17 is cross-linked to a side chain of the stapling amino acid at position 24, and wherein the peptide binds to GLP-1R (SEQ ID NO:5).

In some instances, the stitched peptide comprises the amino acid sequence of SEQ ID NO: 38, wherein each of positions 11, 18, and 25 of the amino acid sequence set forth in SEQ ID NO:38 is replaced with a stapling amino acid, wherein a sidechain of the stapling amino acid at position 11 is cross-linked to a sidechain of the stapling amino acid at position 18 and a sidechain of the stapling amino acid at position 18 is cross-linked to a side chain of the stapling amino acid at position 25, and wherein the peptide binds to GLP-1R (SEQ ID NO:5). In some instances, the stitched peptide comprises the amino acid sequence of SEQ ID NO:31, wherein each of positions 11, 18, and 25 of the amino acid sequence set forth in SEQ ID NO:31 is replaced with a stapling amino acid, wherein a sidechain of the stapling amino acid at position 11 is cross-linked to a sidechain of the stapling amino acid at position 18 and a sidechain of the stapling amino acid at position 18 is cross-linked to a side chain of the stapling amino acid at position 25, and wherein the peptide binds to GLP-1R (SEQ ID NO:5).

In some instances, the stitched peptide comprises the amino acid sequence of SEQ ID NO:38, wherein each of positions 12, 19, and 26 of the amino acid sequence set forth in SEQ ID NO:38 is replaced with a stapling amino acid, wherein a sidechain of the stapling amino acid at position 12 is cross-linked to a sidechain of the stapling amino acid at position 19 and a sidechain of the stapling amino acid at position 19 is cross-linked to a side chain of the stapling amino acid at position 26, and wherein the peptide binds to GLP-1R (SEQ ID NO:5). In some instances, the stitched peptide comprises the amino acid sequence of SEQ ID NO:31, wherein each of positions 12, 19, and 26 of the amino acid sequence set forth in SEQ ID NO:31 is replaced with a stapling amino acid, wherein a sidechain of the stapling amino acid at position 12 is cross-linked to a sidechain of the stapling amino acid at position 19 and a sidechain of the stapling amino acid at position 19 is cross-linked to a side chain of the stapling amino acid at position 26, and wherein the peptide binds to GLP-1R (SEQ ID NO:5).

In some instances, the stitched peptide comprises the amino acid sequence of SEQ ID NO:38, wherein each of positions 6, 13, and 20 of the amino acid sequence set forth in SEQ ID NO:38 is replaced with a stapling amino acid, wherein a sidechain of the stapling amino acid at position 6 is cross-linked to a sidechain of the stapling amino acid at position 13 and a sidechain of the stapling amino acid at position 13 is cross-linked to a side chain of the stapling amino acid at position 20, and wherein the peptide binds to GLP-1R (SEQ ID NO:5). In some instances, the stitched peptide comprises the amino acid sequence of SEQ ID NO:31, wherein each of positions 6, 13, and 20 of the amino acid sequence set forth in SEQ ID NO:31 is replaced with a stapling amino acid, wherein a sidechain of the stapling amino acid at position 6 is cross-linked to a sidechain of the stapling amino acid at position 13 and a sidechain of the stapling amino acid at position 13 is cross-linked to a side chain of the stapling amino acid at position 20, and wherein the peptide binds to GLP-1R (SEQ ID NO:5).

In some instances, the stitched GLP-1 peptide comprises a stitched form of a peptide described in Table 2 (i.e., the stitched peptide is the product of ring-closing metathesis reaction on a peptide of Table 2).

TABLE 2

Exemplary stitched GLP-1 peptides.

| SEQ ID NO | DESCRIPTION | SEQUENCE |
|---|---|---|
| GLP-1 (7-36) | | |
| 61 | SAH-GLP-1(16,23,30) A8J | HJEGTFTSDX₁SSYLEGX₂AAKEFIX₃WLVKGR, wherein each of X₁, X₂, and X₃ is independently a stapling amino acid, and wherein J is 2-aminoisobutyric acid |
| 40 | SAH-GLP-1(16,23,30) A8J | HJEGTFTSD8SSYLEG#AAKEFIZWLVKGR, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine, and J is 2-aminoisobutyric acid |
| 62 | SAH-GLP-1(16,23,30) A8G | HGEGTFTSDX₁SSYLEGX₂AAKEFIX₃WLVKGR, wherein each of X₁, X₂, and X₃ is independently a stapling amino acid |
| 33 | SAH-GLP-1(16,23,30) A8G | HGEGTFTSD8SSYLEG#AAKEFIZWLVKGR, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine |
| 65 | SAH-GLP-1(17,24,31) A8J | HJEGTFTSDVX₁SYLEGQX₂AKEFIAX₃LVKGR, wherein each of X₁, X₂, and X₃ is independently a stapling amino acid, and wherein J is 2-aminoisobutyric acid |
| 41 | SAH-GLP-1(17,24,31) A8J | HJEGTFTSDV8SYLEGQ#AKEFIAZLVKGR, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine, and J is 2-aminoisobutyric acid |
| 66 | SAH-GLP-1(17,24,31) A8G | HGEGTFTSDVX₁SYLEGQX₂AKEFIAX₃LVKGR, wherein each of X₁, X₂, and X₃ is independently a stapling amino acid |
| 34 | SAH-GLP-1(17,24,31) A8G | HGEGTFTSDV8SYLEGQ#AKEFIAZLVKGR, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine |
| 71 | SAH-GLP-1(18,25,32) A8J | HJEGTFTSDVSX₁YLEGQAX₂KEFIAWX₃VKGR, wherein each of X₁, X₂, and X₃ is independently a stapling amino acid, and wherein J is 2-aminoisobutyric acid |
| 72 | SAH-GLP-1(18,25,32) A8J | HJEGTFTSDVS8YLEGQA#KEFIAWZVKGR, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine, and J is 2-aminoisobutyric acid |
| 73 | SAH-GLP-1(18,25,32) A8G | HGEGTFTSDVSX₁YLEGQAX₂KEFIAWX₃VKGR, wherein each of X₁, X₂, and X₃ is independently a stapling amino acid |
| 74 | SAH-GLP-1(18,25,32) A8G | HGEGTFTSDVS8YLEGQA#KEFIAWZVKGR, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine |
| 79 | SAH-GLP-1(12,19,26) A8J | HJEGTX₁TSDVSSX₂LEGQAAX₃EFIAWLVKGR, wherein each of X₁, X₂, and X₃ is independently a stapling amino acid, and wherein J is 2-aminoisobutyric acid |
| 80 | SAH-GLP-1(12,19,26) A8J | HJEGT8TSDVSS#LEGQAAZEFIAWLVKGR, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine, and J is 2-aminoisobutyric acid |
| 81 | SAH-GLP-1(12,19,26) A8G | HGEGTX₁TSDVSSX₂LEGQAAX₃EFIAWLVKGR, wherein each of X₁, X₂, and X₃ is independently a stapling amino acid |
| 82 | SAH-GLP-1(12,19,26) A8G | HGEGT8TSDVSS#LEGQAAZEFIAWLVKGR, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine |
| GLP-1 (7–37) | | |
| 63 | SAH-GLP-1(16,23,30) A8J | HJEGTFTSDX₁SSYLEGX₂AAKEFIX₃WLVKGRG, wherein each of X₁, X₂, and X₃ is independently a stapling amino acid, and wherein J is 2-aminoisobutyric acid |
| 58 | SAH-GLP-1(16,23,30) A8J | HJEGTFTSD8SSYLEG#AAKEFIZWLVKGRG, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine, and J is 2-aminoisobutyric acid |
| 64 | SAH-GLP-1(16,23,30) A8G | HGEGTFTSDX₁SSYLEGX₂AAKEFIX₃WLVKGRG, wherein each of X₁, X₂, and X₃ is independently a stapling amino acid |
| 57 | SAH-GLP-1(16,23,30) A8G | HGEGTFTSD8SSYLEG#AAKEFIZWLVKGRG, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine |
| 67 | SAH-GLP-1(17,24,31) A8J | HJEGTFTSDVX₁SYLEGQX₂AKEFIAX₃LVKGRG, wherein each of X₁, X₂, and X₃ is independently a stapling amino acid, and wherein J is 2-aminoisobutyric acid |
| 60 | SAH-GLP-1(17,24,31) A8J | HJEGTFTSDV8SYLEGQ#AKEFIAZLVKGRG, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine, and J is 2-aminoisobutyric acid |
| 68 | SAH-GLP-1(17,24,31) A8G | HGEGTFTSDVX₁SYLEGQX₂AKEFIAX₃LVKGRG, wherein each of X₁, X₂, and X₃ is independently a stapling amino acid |
| 59 | SAH-GLP-1(17,24,31) A8G | HGEGTFTSDV8SYLEGQ#AKEFIAZLVKGRG, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine |
| 75 | SAH-GLP-1(18,25,32) A8J | HJEGTFTSDVSX₁YLEGQAX₂KEFIAWX₃VKGRG, wherein each of X₁, X₂, and X₃ is independently a stapling amino acid, and wherein J is 2-aminoisobutyric acid |
| 76 | SAH-GLP-1(18,25,32) A8J | HJEGTFTSDVS8YLEGQA#KEFIAWZVKGRG, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine, and J is 2-aminoisobutyric acid |
| 77 | SAH-GLP-1(18,25,32) A8G | HGEGTFTSDVSX₁YLEGQAX₂KEFIAWX₃VKGRG, wherein each of X₁, X₂, and X₃ is independently a stapling amino acid |
| 78 | SAH-GLP-1(18,25,32) A8G | HGEGTFTSDVS8YLEGQA#KEFIAWZVKGRG, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine |
| 83 | SAH-GLP-1(12,19,26) A8J | HJEGTX₁TSDVSSX₂LEGQAAX₃EFIAWLVKGRG, wherein each of X₁, X₂, and X₃ is independently a stapling amino acid, and wherein J is 2-aminoisobutyric acid |
| 84 | SAH-GLP-1(12,19,26) A8J | HJEGT8TSDVSS#LEGQAAZEFIAWLVKGRG, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine, and J is 2-aminoisobutyric acid |
| 85 | SAH-GLP-1(12,19,26) A8G | HGEGTX₁TSDVSSX₂LEGQAAX₃EFIAWLVKGRG, wherein each of X₁, X₂, and X₃ is independently a stapling amino acid |
| 86 | SAH-GLP-1(12,19,26) A8G | HGEGT8TSDVSS#LEGQAAZEFIAWLVKGRG, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine |

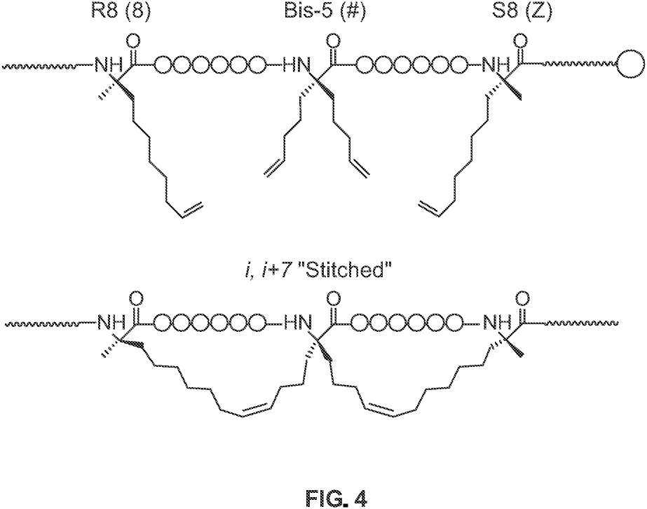

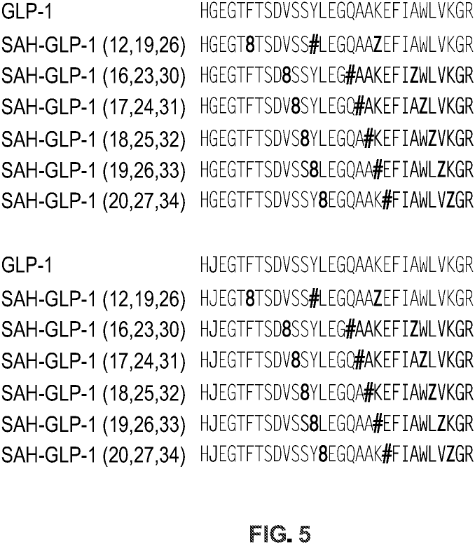

of non-natural amino acids ((R)-α-(7′-octenyl)alanine, α,α-Bis(4′-pentenyl)glycine, and (S)-α-(7′-octenyl)alanine) that can be used to generate various cross-linked compounds. FIG. 4 bottom panel illustrates a peptide with an [i,i+7, i+14] stitch. FIG. 5 shows various GLP-1 peptide sequences with an [i, i+7, i+14] stitch.

In some instances, the disclosure features internally cross-linked (“stitched”) peptides comprising or consisting of the amino acid sequence of any one of SEQ ID NOs:61-64 (or a modified version thereof), wherein a sidechain of the amino acid of position 10 of the amino acid sequence of any one of SEQ ID NOs:61-64 is cross-linked to a sidechain of the amino acid of position 17 of the amino acid sequence of any one of SEQ ID NOs:61-64, respectively, and a sidechain of the amino acid of position 17 of the amino acid sequence of any one of SEQ ID NOs:61-64, respectively, is cross-linked to a sidechain of the amino acid of position 24 of the amino acid sequence of any one of SEQ ID NOs:61-64, respectively, thereby forming a stitch between positions 10, 17, and 24 of the amino acid sequence of any one of SEQ ID NOs:61-64, respectively. In some instances, the disclosure feature an internally cross-linked (“stitched”) peptide comprising the amino acid sequence of SEQ ID NO:61 (or a modified version thereof), wherein a sidechain of the amino acid of position 10 of the amino acid sequence set forth in SEQ ID NO:61 is cross-linked to a sidechain of the amino acid of position 17 of the amino acid sequence set forth in SEQ ID NO:61 and a sidechain of the amino acid of position 17 of the amino acid sequence set forth in SEQ ID NO:61 is cross-linked to a sidechain of the amino acid of position 24 of the amino acid sequence set forth in SEQ ID NO:61, thereby forming a stitch between positions 10, 17, and 24 of the amino acid sequence set forth in SEQ ID NO:61. In some instances, the disclosure features an internally cross-linked (“stitched”) peptide consisting of the amino acid sequence of SEQ ID NO:61 (or a modified version thereof), wherein a sidechain of the amino acid of position 10 of the amino acid sequence set forth in SEQ ID NO:61 is cross-linked to a sidechain of the amino acid of position 17 of the amino acid sequence set forth in SEQ ID NO:61 and a sidechain of the amino acid of position 17 of the amino acid sequence set forth in SEQ ID NO:61 is cross-linked to a sidechain of the amino acid of position 24 of the amino acid sequence set forth in SEQ ID NO:61, thereby forming a stitch between positions 10, 17, and 24 of the amino acid sequence set forth in SEQ ID NO:61. In some instances, the disclosure feature an internally cross-linked (“stitched”) peptide comprising the amino acid sequence of SEQ ID NO:62 (or a modified version thereof), wherein a sidechain of the amino acid of position 10 of the amino acid sequence set forth in SEQ ID NO:62 is cross-linked to a sidechain of the amino acid of position 17 of the amino acid sequence set forth in SEQ ID NO:62 and a sidechain of the amino acid of position 17 of the amino acid sequence set forth in SEQ ID NO:62 is cross-linked to a sidechain of the amino acid of position 24 of the amino acid sequence set forth in SEQ ID NO:62, thereby forming a stitch between positions 10, 17, and 24 of the amino acid sequence set forth in SEQ ID NO:62. In some instances, the disclosure feature an internally cross-linked (“stitched”) peptide comprising the amino acid sequence of SEQ ID NO:63 (or a modified version thereof), wherein a sidechain of the amino acid of position 10 of the amino acid sequence set forth in SEQ ID NO:63 is cross-linked to a sidechain of the amino acid of position 17 of the amino acid sequence set forth in SEQ ID NO:63 and a sidechain of the amino acid of position 17 of the amino acid sequence set forth in SEQ ID NO:63 is cross-linked to a sidechain of the amino acid of position 24 of the amino acid sequence set forth in SEQ ID NO:63, thereby forming a stitch between positions 10, 17, and 24 of the amino acid sequence set forth in SEQ ID NO:63. In some instances, the disclosure feature an internally cross-linked (“stitched”) peptide comprising the amino acid sequence of SEQ ID NO:64 (or a modified version thereof), wherein a sidechain of the amino acid of position 10 of the amino acid sequence set forth in SEQ ID NO:64 is cross-linked to a sidechain of the amino acid of position 17 of the amino acid sequence set forth in SEQ ID NO:64 and a sidechain of the amino acid of position 17 of the amino acid sequence set forth in SEQ ID NO:64 is cross-linked to a sidechain of the amino acid of position 24 of the amino acid sequence set forth in SEQ ID NO:64, thereby forming a stitch between positions 10, 17, and 24 of the amino acid sequence set forth in SEQ ID NO:64. In some instances, X₁ is R-octenyl alanine, X₂ is bis-pentenyl glycine, and X₃ is S-octenyl alanine. In some instances, X₁ is S-octenyl alanine, X₂ is bis-pentenyl glycine, and X₃ is R-octenyl alanine. In some instances, X₁ is (R)-α-(7′-octenyl)alanine, X₂ is α,α-Bis(4′-pentenyl)glycine, and X₃ is (S)-α-(7′-octenyl)alanine. In some instances, X1 is (S)-α-(7′-octenyl)alanine, X2 is α,α-Bis(4′-pentenyl)glycine, and X3 is (R)-α-(7′-octenyl)alanine.

In some instances, the disclosure features internally cross-linked (“stitched”) peptides comprising the amino acid sequence of any one of SEQ ID NOs:65-68 (or a modified version thereof), wherein a sidechain of the amino acid of position 11 of the amino acid sequence of any one of SEQ ID NOs: 65-68 is cross-linked to a sidechain of the amino acid of position 18 of the amino acid sequence of any one of SEQ ID NOs: 65-68, respectively, and a sidechain of the amino acid of position 18 of the amino acid sequence of any one of SEQ ID NOs: 65-68, respectively, is cross-linked to a sidechain of the amino acid of position 25 of the amino acid sequence of any one of SEQ ID NOs: 65-68, respectively, thereby forming a stitch between positions 11, 18, and 25 of the amino acid sequence of any one of SEQ ID NOs:65-68, respectively. In some instances, X₁ is R-octenyl alanine, X₂ is bis-pentenyl glycine, and X₃ is S-octenyl alanine. In some instances, X₁ is S-octenyl alanine, X₂ is bis-pentenyl glycine, and X₃ is R-octenyl alanine. In some instances, X₁ is (R)-α-(7′-octenyl)alanine, X₂ is α,α-Bis(4′-pentenyl)glycine, and X₃ is (S)-α-(7′-octenyl)alanine. In some instances, X₁ is (S)-α-(7′-octenyl)alanine, X₂ is α,α-Bis(4′-pentenyl)glycine, and X₃ is (R)-α-(7′-octenyl)alanine.

In some instances, the disclosure features internally cross-linked (“stitched”) peptides comprising the amino acid sequence of any one of SEQ ID NOs:71, 73, 75, and 77 (or a modified version thereof), wherein a sidechain of the amino acid of position 12 of the amino acid sequence of any one of SEQ ID NOs: 71, 73, 75, and 77 is cross-linked to a sidechain of the amino acid of position 19 of the amino acid sequence of any one of SEQ ID NOs: 71, 73, 75, and 77, respectively, and a sidechain of the amino acid of position 19 of the amino acid sequence of any one of SEQ ID NOs: 71, 73, 75, and 77, respectively, is cross-linked to a sidechain of the amino acid of position 26 of the amino acid sequence of any one of SEQ ID NOs: 71, 73, 75, and 77, respectively, thereby forming a stitch between positions 12, 19, and 26 of the amino acid sequence of any one of SEQ ID NOs:71, 73, 75, and 77, respectively. In some instances, X₁ is R-octenyl alanine, X₂ is bis-pentenyl glycine, and X₃ is S-octenyl alanine. In some instances, X₁ is S-octenyl alanine, X₂ is bis-pentenyl glycine, and X₃ is R-octenyl alanine. In some instances, X₁ is (R)-α-(7′-octenyl)alanine, X₂ is α,α-Bis(4′-pentenyl)glycine, and X₃ is (S)-α-(7′-octenyl)alanine. In some instances, X₁ is (S)-α-(7′-octenyl)alanine, X₂ is α,α-Bis(4′-pentenyl)glycine, and X₃ is (R)-α-(7′-octenyl)alanine.

In some instances, the disclosure features internally cross-linked (“stitched”) peptides comprising the amino acid sequence of any one of SEQ ID NOs:79, 81, 83, and 85 (or a modified version thereof), wherein a sidechain of the amino acid of position 6 of the amino acid sequence of any one of SEQ ID NOs:79, 81, 83, and 85 is cross-linked to a sidechain of the amino acid of position 13 of the amino acid sequence of any one of SEQ ID NOs: 79, 81, 83, and 85, respectively, and a sidechain of the amino acid of position 13 of the amino acid sequence of any one of SEQ ID NOs:79, 81, 83, and 85, respectively, is cross-linked to a sidechain of the amino acid of position 20 of the amino acid sequence of any one of SEQ ID NOs: 79, 81, 83, and 85, respectively, thereby forming a stitch between positions 6, 13, and 20 of the amino acid sequence of any one of SEQ ID NOs:79, 81, 83, and 85, respectively. In some instances, X₁ is R-octenyl alanine, X₂ is bis-pentenyl glycine, and X₃ is S-octenyl alanine. In some instances, X₁ is S-octenyl alanine, X₂ is bis-pentenyl glycine, and X₃ is R-octenyl alanine. In some instances, X₁ is (R)-α-(7′-octenyl)alanine, X₂ is α,α-Bis(4′-pentenyl)glycine, and X₃ is (S)-α-(7′-octenyl)alanine. In some instances, X₁ is (S)-α-(7′-octenyl)alanine, X₂ is α,α-Bis(4′-pentenyl)glycine, and X₃ is (R)-α-(7′-octenyl)alanine.

In some instances, the disclosure features internally cross-linked (“stitched”) peptides comprising or consisting of the amino acid sequence of SEQ ID NO:40, 33, 57, or 58 (or a modified version thereof), wherein a sidechain of the amino acid of position 10 of the amino acid sequence of any one of SEQ ID NOs: 40, 33, 57, or 58, respectively, is cross-linked to a sidechain of the amino acid of position 17 of the amino acid sequence of any one of SEQ ID NOs:40, 33, 57, or 58, respectively, and a sidechain of the amino acid of position 17 of the amino acid sequence of any one of SEQ ID NOs:40, 33, 57, or 58, respectively, is cross-linked to a sidechain of the amino acid of position 24 of the amino acid sequence of any one of SEQ ID NOs:40, 33, 57, or 58, respectively, thereby forming a stitch between positions 10, 17, and 24 of the amino acid sequence of SEQ ID NO: 40, 33, 57, or 58, respectively. In some instances, the disclosure features internally cross-linked (“stitched”) peptides comprising the amino acid sequence of SEQ ID NO: 40, wherein a sidechain of the amino acid of position 10 of the amino acid sequence of SEQ ID NO:40 is cross-linked to a sidechain of the amino acid of position 17 of the amino acid sequence set forth in SEQ ID NO:40 and a sidechain of the amino acid of position 17 of the amino acid sequence set forth in SEQ ID NO:40 is cross-linked to a sidechain of the amino acid of position 24 of the amino acid sequence set forth in SEQ ID NO:40, thereby forming a stitch between positions 10, 17, and 24 of the amino acid sequence set forth in SEQ ID NO:40. In some instances, the disclosure features internally cross-linked (“stitched”) peptides consisting of the amino acid sequence of SEQ ID NO: 40, wherein a sidechain of the amino acid of position 10 of the amino acid sequence set forth in SEQ ID NO:40 is cross-linked to a sidechain of the amino acid of position 17 of the amino acid sequence set forth in SEQ ID NO:40 and a sidechain of the amino acid of position 17 of the amino acid sequence set forth in SEQ ID NO:40 is cross-linked to a sidechain of the amino acid of position 24 of the amino acid sequence set forth in SEQ ID NO:40, thereby forming a stitch between positions 10, 17, and 24 of the amino acid sequence set forth in SEQ ID NO:40. In some instances, the disclosure features internally cross-linked (“stitched”) peptides comprising or consisting of the amino acid sequence of SEQ ID NO:33, wherein a sidechain of the amino acid of position 10 of the amino acid sequence set forth in SEQ ID NO:33 is cross-linked to a sidechain of the amino acid of position 17 of the amino acid sequence set forth in SEQ ID NO:33 and a sidechain of the amino acid of position 17 of the amino acid sequence set forth in SEQ ID NO:33 is cross-linked to a sidechain of the amino acid of position 24 of the amino acid sequence set forth in SEQ ID NO:33. In some instances, the disclosure features internally cross-linked (“stitched”) peptides comprising or consisting of the amino acid sequence of SEQ ID NO:57, wherein a sidechain of the amino acid of position 10 of the amino acid sequence set forth in SEQ ID NO:57 is cross-linked to a sidechain of the amino acid of position 17 of the amino acid sequence set forth in SEQ ID NO:57 and a sidechain of the amino acid of position 17 of the amino acid sequence set forth in SEQ ID NO:57 is cross-linked to a sidechain of the amino acid of position 24 of the amino acid sequence set forth in SEQ ID NO:57, thereby forming a stitch between positions 10, 17, and 24 of the amino acid sequence set forth in SEQ ID NO:57. In some instances, the disclosure features internally cross-linked (“stitched”) peptides comprising or consisting of the amino acid sequence of SEQ ID NO:58, wherein a sidechain of the amino acid of position 10 of the amino acid sequence set forth in SEQ ID NO:58 is cross-linked to a sidechain of the amino acid of position 17 of the amino acid sequence set forth in SEQ ID NO:58 and a sidechain of the amino acid of position 17 of the amino acid sequence set forth in SEQ ID NO:58 is cross-linked to a sidechain of the amino acid of position 24 of the amino acid sequence set forth in SEQ ID NO:58, thereby forming a stitch between positions 10, 17, and 24 of the amino acid sequence set forth in SEQ ID NO:58.

In some instances, the disclosure features internally cross-linked (“stitched”) peptides comprising or consisting of the amino acid sequence of any one of SEQ ID NOs:34, 41, 59, or 60 (or a modified version thereof), wherein a sidechain of the amino acid of position 11 of the amino acid sequence of any one of SEQ ID NOs: 34, 41, 59, or 60, respectively, is cross-linked to a sidechain of the amino acid of position 18 of the amino acid sequence of any one of SEQ ID NOs: 34, 41, 59, or 60, respectively, and a sidechain of the amino acid of position 18 of the amino acid sequence of any one of SEQ ID NOs: 34, 41, 59, or 60, respectively, is cross-linked to a sidechain of the amino acid of position 25 of the amino acid sequence of any one of SEQ ID NOs: 34, 41, 59, or 60, respectively, thereby forming a stitch between positions 11, 18, and 25 of the amino acid sequence set forth in SEQ ID NO:34, 41, 59, or 60, respectively. In some instances, the disclosure features internally cross-linked (“stitched”) peptides comprising or consisting of the amino acid sequence of SEQ ID NO:34, wherein a sidechain of the amino acid of position 11 of the amino acid sequence set forth in SEQ ID NO:34 is cross-linked to a sidechain of the amino acid of position 18 of the amino acid sequence set forth in SEQ ID NO:34 and a sidechain of the amino acid of position 18 of the amino acid sequence set forth in SEQ ID NO:34 is cross-linked to a sidechain of the amino acid of position 25 of the amino acid sequence set forth in SEQ ID NO:34, thereby forming a stitch between positions 11, 18, and 25 of the amino acid sequence set forth in SEQ ID NO:34. In some instances, the disclosure features internally cross-linked (“stitched”) peptides comprising or consisting of the amino acid sequence of SEQ ID NO:41, wherein a sidechain of the amino acid of position 11 of the amino acid sequence set forth in SEQ ID NO:41 is cross-linked to a sidechain of the amino acid of position 18 of the amino acid sequence set forth in SEQ ID NO:41 and a sidechain of the amino acid of position 18 of the amino acid sequence set forth in SEQ ID NO:41 is cross-linked to a sidechain of the amino acid of position 25 of the amino acid sequence set forth in SEQ ID NO:41, thereby forming a stitch between positions 11, 18, and 25 of the amino acid sequence set forth in SEQ ID NO:41. In some instances, the disclosure features internally cross-linked (“stitched”) peptides comprising or consisting of the amino acid sequence of SEQ ID NO:59, wherein a sidechain of the amino acid of position 11 of the amino acid sequence set forth in SEQ ID NO:59 is cross-linked to a sidechain of the amino acid of position 18 of the amino acid sequence set forth in SEQ ID NO:59 and a sidechain of the amino acid of position 18 of the amino acid sequence set forth in SEQ ID NO:59 is cross-linked to a sidechain of the amino acid of position 25 of the amino acid sequence set forth in SEQ ID NO:59, thereby forming a stitch between positions 11, 18, and 25 of the amino acid sequence set forth in SEQ ID NO:59. In some instances, the disclosure features internally cross-linked (“stitched”) peptides comprising or consisting of the amino acid sequence of SEQ ID NO:60, wherein a sidechain of the amino acid of position 11 of the amino acid sequence set forth in SEQ ID NO:60 is cross-linked to a sidechain of the amino acid of position 18 of the amino acid sequence set forth in SEQ ID NO:60 and a sidechain of the amino acid of position 18 of the amino acid sequence set forth in SEQ ID NO:60 is cross-linked to a sidechain of the amino acid of position 25 of the amino acid sequence set forth in SEQ ID NO:60, thereby forming a stitch between positions 11, 18, and 25 of the amino acid sequence set forth in SEQ ID NO:60.

In some instances, the disclosure features internally cross-linked (“stitched”) peptides comprising or consisting of the amino acid sequence of any one of SEQ ID NOs:72, 74, 76, and 78 (or a modified version thereof), wherein a sidechain of the amino acid of position 12 of the amino acid sequence of any one of SEQ ID NOs: 72, 74, 76, and 78, respectively, is cross-linked to a sidechain of the amino acid of position 19 of the amino acid sequence of any one of SEQ ID NOs: 72, 74, 76, and 78, respectively, and a sidechain of the amino acid of position 19 of the amino acid sequence of any one of SEQ ID NOs: 72, 74, 76, and 78, respectively, is cross-linked to a sidechain of the amino acid of position 26 of the amino acid sequence of any one of SEQ ID NOs: 72, 74, 76, and 78, respectively, thereby forming a stitch between positions 12, 19, and 26 of the amino acid sequence set forth in SEQ ID NO: 72, 74, 76, and 78, respectively. In some instances, the disclosure features internally cross-linked (“stitched”) peptides comprising or consisting of the amino acid sequence of SEQ ID NO:72, wherein a sidechain of the amino acid of position 12 of the amino acid sequence set forth in SEQ ID NO:72 is cross-linked to a sidechain of the amino acid of position 19 of the amino acid sequence set forth in SEQ ID NO:72 and a sidechain of the amino acid of position 19 of the amino acid sequence set forth in SEQ ID NO:72 is cross-linked to a sidechain of the amino acid of position 26 of the amino acid sequence set forth in SEQ ID NO:72, thereby forming a stitch between positions 12, 19, and 26 of the amino acid sequence set forth in SEQ ID NO:72. In some instances, the disclosure features internally cross-linked (“stitched”) peptides comprising or consisting of the amino acid sequence of SEQ ID NO:74, wherein a sidechain of the amino acid of position 12 of the amino acid sequence set forth in SEQ ID NO:74 is cross-linked to a sidechain of the amino acid of position 19 of the amino acid sequence set forth in SEQ ID NO:74 and a sidechain of the amino acid of position 19 of the amino acid sequence set forth in SEQ ID NO:74 is cross-linked to a sidechain of the amino acid of position 26 of the amino acid sequence set forth in SEQ ID NO:74, thereby forming a stitch between positions 12, 19, and 26 of the amino acid sequence set forth in SEQ ID NO:74. In some instances, the disclosure features internally cross-linked (“stitched”) peptides comprising or consisting of the amino acid sequence of SEQ ID NO:76, wherein a sidechain of the amino acid of position 12 of the amino acid sequence set forth in SEQ ID NO:76 is cross-linked to a sidechain of the amino acid of position 19 of the amino acid sequence set forth in SEQ ID NO:76 and a sidechain of the amino acid of position 19 of the amino acid sequence set forth in SEQ ID NO:76 is cross-linked to a sidechain of the amino acid of position 26 of the amino acid sequence set forth in SEQ ID NO:76, thereby forming a stitch between positions 12, 19, and 26 of the amino acid sequence set forth in SEQ ID NO:76. In some instances, the disclosure features internally cross-linked (“stitched”) peptides comprising or consisting of the amino acid sequence of SEQ ID NO:78, wherein a sidechain of the amino acid of position 12 of the amino acid sequence set forth in SEQ ID NO:78 is cross-linked to a sidechain of the amino acid of position 19 of the amino acid sequence set forth in SEQ ID NO:78 and a sidechain of the amino acid of position 19 of the amino acid sequence set forth in SEQ ID NO:78 is cross-linked to a sidechain of the amino acid of position 26 of the amino acid sequence set forth in SEQ ID NO:78, thereby forming a stitch between positions 12, 19, and 26 of the amino acid sequence set forth in SEQ ID NO:78.

In some instances, the disclosure features internally cross-linked (“stitched”) peptides comprising or consisting of the amino acid sequence of any one of SEQ ID NOs:80, 82, 84, and 86 (or a modified version thereof), wherein a sidechain of the amino acid of position 6 of the amino acid sequence of any one of SEQ ID NOs: 80, 82, 84, and 86, respectively, is cross-linked to a sidechain of the amino acid of position 13 of the amino acid sequence of any one of SEQ ID NOs: 80, 82, 84, and 86, respectively, and a sidechain of the amino acid of position 13 of the amino acid sequence of any one of SEQ ID NOs: 80, 82, 84, and 86, respectively, is cross-linked to a sidechain of the amino acid of position 20 of the amino acid sequence of any one of SEQ ID NOs: 80, 82, 84, and 86, respectively, thereby forming a stitch between positions 6, 13, and 20 of the amino acid sequence of SEQ ID NO: 80, 82, 84, and 86, respectively. In some instances, the disclosure features internally cross-linked (“stitched”) peptides comprising or consisting of the amino acid sequence of SEQ ID NO:80, wherein a sidechain of the amino acid of position 6 of the amino acid sequence set forth in SEQ ID NO:80 is cross-linked to a sidechain of the amino acid of position 13 of the amino acid sequence set forth in SEQ ID NO:80 and a sidechain of the amino acid of position 13 of the amino acid sequence set forth in SEQ ID NO:80 is cross-linked to a sidechain of the amino acid of position 20 of the amino acid sequence set forth in SEQ ID NO:80, thereby forming a stitch between positions 6, 13, and 20 of the amino acid sequence set forth in SEQ ID NO:80. In some instances, the disclosure features internally cross-linked (“stitched”) peptides comprising or consisting of the amino acid sequence of SEQ ID NO:82, wherein a sidechain of the amino acid of position 6 of the amino acid sequence set forth in SEQ ID NO:82 is cross-linked to a sidechain of the amino acid of position 13 of the amino acid sequence set forth in SEQ ID NO:82 and a sidechain of the amino acid of position 13 of the amino acid sequence set forth in SEQ ID NO:82 is cross-linked to a sidechain of the amino acid of position 20 of the amino acid sequence set forth in SEQ ID NO:82, thereby forming a stitch between positions 6, 13, and 20 of the amino acid sequence set forth in SEQ ID NO:82. In some instances, the disclosure features internally cross-linked (“stitched”) peptides comprising or consisting of the amino acid sequence of SEQ ID NO:84, wherein a sidechain of the amino acid of position 6 of the amino acid sequence set forth in SEQ ID NO:84 is cross-linked to a sidechain of the amino acid of position 13 of the amino acid sequence set forth in SEQ ID NO:84 and a sidechain of the amino acid of position 13 of the amino acid sequence set forth in SEQ ID NO:84 is cross-linked to a sidechain of the amino acid of position 20 of the amino acid sequence set forth in SEQ ID NO:84, thereby forming a stitch between positions 6, 13, and 20 of the amino acid sequence set forth in SEQ ID NO:84. In some instances, the disclosure features internally cross-linked (“stitched”) peptides comprising or consisting of the amino acid sequence of SEQ ID NO:86, wherein a sidechain of the amino acid of position 6 of the amino acid sequence set forth in SEQ ID NO:86 is cross-linked to a sidechain of the amino acid of position 13 of the amino acid sequence set forth in SEQ ID NO:86 and a sidechain of the amino acid of position 13 of the amino acid sequence set forth in SEQ ID NO:86 is cross-linked to a sidechain of the amino acid of position 20 of the amino acid sequence set forth in SEQ ID NO:86, thereby forming a stitch between positions 6, 13, and 20 of the amino acid sequence set forth in SEQ ID NO:86.

In one aspect, provided herein is a :

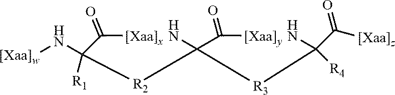

wherein:
each R₁ and R₄ is independently H or a C₁₋₁₀ alkyl, alkenyl, alkynyl, arylalkyl, cycloalkylalkyl, heteroarylalkyl, or heterocyclylalkyl, any of which is substituted or unsubstituted;
each of R₂ and R₃ is independently a C₅₋₂₀ alkyl, alkenyl, alkynyl; or [R₄—K—R₄]n; each of which is substituted with 0-6 R₅;
R₅ is halo, alkyl, OR₆, N(R₆)₂, SR₆, SOR₆, SO₂R₆, CO₂R₆, R₆, a fluorescent moiety, or a radioisotope;
K is O, S, SO, SO₂, CO, CO₂, CONR₆, or

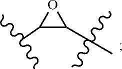

R₆ is H, alkyl, or a therapeutic agent;
n is an integer from 1-4;
[Xaa]_{w} is 1, 2, 3, 4, 5, 6, 7, 8, 9, or 10 amino acids;
[Xaa]z is 1, 2, 3, 4, 5, 6, or 7 amino acids;
   (a) [Xaa]ₓ is SSYLEG (SEQ ID NO:46) and [Xaa]_{y} is AAKEFI (SEQ ID NO:47),
   (b) [Xaa]ₓ is SYLEGQ (SEQ ID NO:51) and [Xaa]_{y} is AKEFIA (SEQ ID NO:52),
   (c) [Xaa]ₓ is TSDVSS (SEQ ID NO:87) and [Xaa]_{y} is LEGQAA (SEQ ID NO:88), or
   (d) [Xaa]ₓ is YLEGQA (SEQ ID NO:89) and [Xaa]_{y} is KEFIAW (SEQ ID NO:90); and
the amino acid sequence has at least 80% or at least 85% identity to the amino acid sequence of SEQ ID NO:3 or 4. In some instances, [Xaa]ₓ is SSYLEG (SEQ ID NO:46) and [Xaa]_{y} is AAKEFI (SEQ ID NO:47).

In some instances of Formula (I),
[Xaa]_{w} is HJEGTFTSD (SEQ ID NO:45) or HGEGTFTSD (SEQ ID NO:49), [Xaa]ₓ is SSYLEG (SEQ ID NO:46), [Xaa]_{y} is AAKEFI (SEQ ID NO:47), [Xaa]_{z} is WLVKGR (SEQ ID NO:48), wherein J is 2-aminoisobutyric acid,
each R₁ and R₄ is independently H, alkyl, alkenyl, alkynyl, arylalkyl, cycloalkylalkyl, heteroarylalkyl, or heterocyclylalkyl, any of which is substituted or unsubstituted; each R₂ and R₃ is independently alkylene, alkenylene, or alkynylene, any of which is substituted or unsubstituted;
the stitched amino acid sequence binds to GLP-1R (SEQ ID NO:5), and the cross-linked amino acid sequence has an alpha helical conformation. In some instances of Formula (I), R₁ and R₄ are independently H or a C₁₋₁₀ alkyl, alkenyl, alkynyl, arylalkyl, cycloalkylalkyl, heteroarylalkyl, or heterocyclylalkyl. In some instances of Formula (I), R₂ and R₃ are independently a C₅₋₂₀ alkyl, alkenyl, alkynyl; or [R₄—K—R₄]n; each of which is substituted with 0-6 R₅, wherein R₅ is halo, alkyl, OR₆,
N(R₆)₂, SR₆, SOR₆, SO₂R₆, CO₂R₆, R₆, a fluorescent moiety, or a radioisotope; K is O,
S, SO, SO₂, CO, CO₂, CONR₆, or

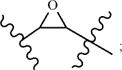

R₆ is H, alkyl, or a therapeutic agent; and
n is an integer from 1-4. In some instances of Formula (I), R₁ is an alkyl. In some instances of Formula (I), R₁ is a methyl group. In some instances of Formula (I), R₄ is an alkyl. In some instances of Formula (I), R₄ is a methyl group. In some instances of Formula (I), R₂ is an alkenyl. In some instances of Formula (I), R₃ is an alkenyl. In some instances of Formula (I), R₁ is a methyl group, R₂ is (CH₂)₆-CH=CH-(CH₂)₃, R₃ is(CH₂)₃-CH=CH-(CH₂)₆, and R₄ is a methyl group. In some instances of Formula (I), R₁ is a methyl group, R₂ is (CH₂)₃—CH=CH—(CH₂)₆, R₃ is (CH₂)₆—CH=CH-(CH₂)₃, and R₄ is a methyl group.

In some instances of Formula (I),
[Xaa]_{w} is HJEGTFTSD (SEQ ID NO:45) or HGEGTFTSD (SEQ ID NO:49), [Xaa]ₓ is SSYLEG (SEQ ID NO:46), [Xaa]_{y} is AAKEFI (SEQ ID NO:47), [Xaa]_{z} is WLVKGRG (SEQ ID NO:55), wherein J is 2-aminoisobutyric acid,
each R₁ and R₄ is independently H, alkyl, alkenyl, alkynyl, arylalkyl, cycloalkylalkyl, heteroarylalkyl, or heterocyclylalkyl, any of which is substituted or unsubstituted;
each R₂ and R₃ is independently alkylene, alkenylene, or alkynylene, any of which is substituted or unsubstituted;
the stitched amino acid sequence binds to GLP-1R (SEQ ID NO:5), and the cross-linked amino acid sequence has an alpha helical conformation. In some instances of Formula (I), R₁ and R₄ are independently H or a C₁-₁₀ alkyl, alkenyl, alkynyl, arylalkyl, cycloalkylalkyl, heteroarylalkyl, or heterocyclylalkyl. In some instances of Formula (I), R₂ and R₃ are independently a C₅-₂₀ alkyl, alkenyl, alkynyl; or [R₄—K—R₄]n; each of which is substituted with 0-6 R₅, wherein R₅ is halo, alkyl, OR₆, N(R₆)₂, SR₆, SOR₆, SO₂R₆, CO₂R₆, R₆, a fluorescent moiety, or a radioisotope; K is O,
S, SO, SO₂, CO, CO₂, CONR₆, or

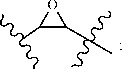

R₆ is H, alkyl, or a therapeutic agent; and
n is an integer from 1-4. In some instances of Formula (I), R₁ is an alkyl. In some instances of Formula (I), R₁ is a methyl group. In some instances of Formula (I), R₄ is an alkyl. In some instances of Formula (I), R₄ is a methyl group. In some instances of Formula (I), R₂ is an alkenyl. In some instances of Formula (I), R₃ is an alkenyl. In some instances of Formula (I), R₁ is a methyl group, R₂ is (CH₂)₆-CH=CH-(CH₂)₃, R₃ is (CH₂)₃-CH=CH-(CH₂)₆, and R₄ is a methyl group. In some instances of Formula (I), R₁ is a methyl group, R₂ is (CH₂)₃-CH=CH-(CH₂)₆, R₃ is (CH₂)₆-CH=CH-(CH₂)₃, and R₄ is a methyl group.

In some instances of Formula (I),
[Xaa]_{w} is HJEGTFTSDV (SEQ ID NO:50) or HGEGTFTSDV (SEQ ID NO:54), [Xaa]ₓ is SYLEGQ (SEQ ID NO:51), [Xaa]_{y} is AKEFIA (SEQ ID NO:52), and [Xaa]_{z} is LVKGR (SEQ ID NO:53) or LVKGRG (SEQ ID NO:56), wherein J is 2-aminoisobutyric acid,
each R₁ and R₄ is independently H, alkyl, alkenyl, alkynyl, arylalkyl, cycloalkylalkyl, heteroarylalkyl, or heterocyclylalkyl, any of which is substituted or unsubstituted;
each R₂ and R₃ is independently alkylene, alkenylene, or alkynylene, any of which is substituted or unsubstituted;
the stitched amino acid sequence binds to GLP-1R (SEQ ID NO:5), and
the cross-linked amino acid sequence has an alpha helical conformation. In some instances of Formula (I), R₁ and R₄ are independently H or a C₁₋₁₀ alkyl, alkenyl, alkynyl, arylalkyl, cycloalkylalkyl, heteroarylalkyl, or heterocyclylalkyl. In some instances of Formula (I), R₂ and R₃ are independently a C₅₋₂₀ alkyl, alkenyl, alkynyl; or [R₄—K—R₄]n; each of which is substituted with 0-6 R₅, wherein R₅ is halo, alkyl, OR₆, N(R₆)₂, SR₆, SOR₆, SO₂R₆, CO₂R₆, R₆, a fluorescent moiety, or a radioisotope; K is O,
S, SO, SO₂, CO, CO₂, CONR₆, or

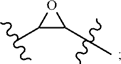

R₆ is H, alkyl, or a therapeutic agent; and
n is an integer from 1-4. In some instances of Formula (I), R₁ is an alkyl. In some instances of Formula (I), R₁ is a methyl group. In some instances of Formula (I), R₄ is an alkyl. In some instances of Formula (I), R₄ is a methyl group. In some instances of Formula (I), R₂ is an alkenyl. In some instances of Formula (I), R₃ is an alkenyl. In some instances of Formula (I), R₁ is a methyl group, R₂ is (CH₂)₆-CH=CH-(CH₂)₃, R₃ is (CH₂)₃-CH=CH-(CH₂)₆, and R₄ is a methyl group. In some instances of Formula (I), R₁ is a methyl group, R₂ is (CH₂)₃-CH=CH-(CH₂)₆, R₃ is (CH₂)₆-CH=CH-(CH₂)₃, and R₄ is a methyl group.

In some instances of Formula (I),
[Xaa]_{w} is HGEGT (SEQ ID NO:91) or HJEGT (SEQ ID NO:92), [Xaa]ₓ is TSDVSS (SEQ ID NO:87), [Xaa]_{y} is LEGQAA (SEQ ID NO:88), and [Xaa]_{z} is EFIAWLVKGR (SEQ ID NO:93) or EFIAWLVKGRG (SEQ ID NO:94), wherein J is 2-aminoisobutryic acid,
each R₁ and R₄ is independently H, alkyl, alkenyl, alkynyl, arylalkyl, cycloalkylalkyl, heteroarylalkyl, or heterocyclylalkyl, any of which is substituted or unsubstituted;
each R₂ and R₃ is independently alkylene, alkenylene, or alkynylene, any of which is substituted or unsubstituted;
the stitched amino acid sequence binds to GLP-1R (SEQ ID NO:5), and the cross-linked amino acid sequence has an alpha helical conformation. In some instances of Formula (I), R₁ and R₄ are independently H or a C₁-₁₀ alkyl, alkenyl, alkynyl, arylalkyl, cycloalkylalkyl, heteroarylalkyl, or heterocyclylalkyl. In some instances of Formula (I), R₂ and R₃ are independently a C₅-₂₀ alkyl, alkenyl, alkynyl; or [R₄—K—R₄]n; each of which is substituted with 0-6 R₅, wherein R₅ is halo, alkyl, OR₆, N(R₆)₂, SR₆, SOR₆, SO₂R₆, CO₂R₆, R₆, a fluorescent moiety, or a radioisotope; K is O,
S, SO, SO₂, CO, CO₂, CONR₆, or

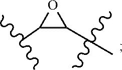

R₆ is H, alkyl, or a therapeutic agent; and
n is an integer from 1-4. In some instances of Formula (I), R₁ is an alkyl. In some instances of Formula (I), wherein R₁ is a methyl group. In some instances of Formula (I), R₄ is an alkyl. In some instances of Formula (I), R₄ is a methyl group. In some instances of Formula (I), R₂ is an alkenyl. In some instances of Formula (I), R₃ is an alkenyl. In some instances of Formula (I), R₁ is a methyl group, R₂ is (CH₂)₆-CH=CH-(CH₂)₃, R₃ is (CH₂)₃-CH=CH-(CH₂)₆, and R₄ is a methyl group. In some instances of Formula (I), R₁ is a methyl group, R₂ is (CH₂)₃-CH=CH-(CH₂)₆, R₃ is (CH₂)₆-CH=CH-(CH₂)₃, and R₄ is a methyl group.

In some instances of Formula (I),
[Xaa]_{w} is HGEGTFTSDVS (SEQ ID NO:95) or HJEGTFTSDVS (SEQ ID NO:96),
[Xaa]ₓ is YLEGQA (SEQ ID NO:89), [Xaa]_{y} is KEFIAW (SEQ ID NO:90), and [Xaa]_{z} is VKGR (SEQ ID NO:97) or VKGRG (SEQ ID NO:98), wherein J is 2-aminoisobutryic acid,
each R₁ and R₄ is independently H, alkyl, alkenyl, alkynyl, arylalkyl, cycloalkylalkyl, heteroarylalkyl, or heterocyclylalkyl, any of which is substituted or unsubstituted; each R₂ and R₃ is independently alkylene, alkenylene, or alkynylene, any of which is substituted or unsubstituted;
the stitched amino acid sequence binds to GLP-1R (SEQ ID NO:5), and the cross-linked amino acid sequence has an alpha helical conformation. In some instances of Formula (I), R₁ and R₄ are independently H or a C₁-₁₀ alkyl, alkenyl, alkynyl, arylalkyl, cycloalkylalkyl, heteroarylalkyl, or heterocyclylalkyl. In some instances of Formula (I), R₂ and R₃ are independently a C₅-₂₀ alkyl, alkenyl, alkynyl; or [R₄—K—R₄]n; each of which is substituted with 0-6 R₅, wherein R₅ is halo, alkyl, OR₆, N(R₆)₂, SR₆, SOR₆, SO₂R₆, CO₂R₆, R₆, a fluorescent moiety, or a radioisotope; K is O,
S, SO, SO₂, CO, CO₂, CONR₆, or

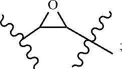

R₆ is H, alkyl, or a therapeutic agent; and
n is an integer from 1-4. In some instances of Formula (I), wherein R₁ is an alkyl. In some instances of Formula (I), R₁ is a methyl group. In some instances of Formula (I),
R₄ is an alkyl. In some instances of Formula (I), R₄ is a methyl group. In some instances of Formula (I), R₂ is an alkenyl. In some instances of Formula (I), R₃ is an alkenyl. In some instances of Formula (I), R₁ is a methyl group, R₂ is (CH₂)₆-CH=CH-(CH₂)₃, R₃ is (CH₂)₃-CH=CH-(CH₂)₆, and R₄ is a methyl group. In some instances of Formula (I), R₁ is a methyl group, R₂ is (CH₂)₃-CH=CH-(CH₂)₆, R₃ is (CH₂)₆-CH=CH-(CH₂)₃, and R₄ is a methyl group.

In some instances of Formula (I), [Xaa]_{w} is HJEGTFTSD (SEQ ID NO:45), [Xaa]ₓ is SSYLEG (SEQ ID NO:46), [Xaa]_{y} is AAKEFI (SEQ ID NO:47), and [Xaa]_{z} is WLVKGR (SEQ ID NO:48), wherein J is 2-aminoisobutryic acid. In some instances of Formula (I), [Xaa]_{w} is HJEGTFTSD (SEQ ID NO:45), [Xaa]ₓ is SSYLEG (SEQ ID NO:46), [Xaa]_{y} is AAKEFI (SEQ ID NO:47), and [Xaa]_{z} is WLVKGRG (SEQ ID NO:55), wherein J is 2-aminoisobutryic acid. In some instances of Formula (I), [Xaa]_{w} is HGEGTFTSD (SEQ ID NO:49), [Xaa]ₓ is SSYLEG (SEQ ID NO:46), [Xaa]_{y} is AAKEFI (SEQ ID NO:47), and [Xaa]_{z} is WLVKGR (SEQ ID NO:48). In some instances of Formula (I), [Xaa]_{w} is HGEGTFTSD (SEQ ID NO:49), [Xaa]ₓ is SSYLEG (SEQ ID NO:46), [Xaa]_{y} is AAKEFI (SEQ ID NO:47), and [Xaa]_{z} is WLVKGRG (SEQ ID NO:55).

In some instances of Formula (I), [Xaa]_{w} is HJEGTFTSDV (SEQ ID NO:50), [Xaa]ₓ is SYLEGQ (SEQ ID NO:51), [Xaa]_{y} is AKEFIA (SEQ ID NO:52), and [Xaa]_{z} is LVKGR (SEQ ID NO:53), wherein J is 2-aminoisobutryic acid. In some instances of Formula (I), [Xaa]_{w} is HJEGTFTSDV (SEQ ID NO:50), [Xaa]ₓ is SYLEGQ (SEQ ID NO:51), [Xaa]_{y} is AKEFIA (SEQ ID NO:52), and [Xaa]_{z} is LVKGRG (SEQ ID NO:56), wherein J is 2-aminoisobutryic acid. In some instances of Formula (I), [Xaa]_{w} is HGEGTFTSDV (SEQ ID NO:54), [Xaa]ₓ is SYLEGQ (SEQ ID NO:51), [Xaa]_{y} is AKEFIA (SEQ ID NO:52), and [Xaa]_{z} is LVKGR (SEQ ID NO:53). In some instances of Formula (I), [Xaa]_{w} is HGEGTFTSDV (SEQ ID NO:54), [Xaa]ₓ is SYLEGQ (SEQ ID NO:51), [Xaa]_{y} is AKEFIA (SEQ ID NO:52), and [Xaa]_{z} is LVKGRG (SEQ ID NO:56).

In some instances of Formula (I), [Xaa]_{w} is HGEGT (SEQ ID NO:91) or HJEGT (SEQ ID NO:92), [Xaa]ₓ is TSDVSS (SEQ ID NO:87), [Xaa]_{y} is LEGQAA (SEQ ID NO:88), and [Xaa]_{z} is EFIAWLVKGR (SEQ ID NO:93) or EFIAWLVKGRG (SEQ ID NO:94), wherein J is 2-aminoisobutryic acid.

In some instances of Formula (I), [Xaa]_{w} is HGEGTFTSDVS (SEQ ID NO:95) or HJEGTFTSDVS (SEQ ID NO:96), [Xaa]ₓ is YLEGQA (SEQ ID NO:89), [Xaa]_{y} is KEFIAW (SEQ ID NO:90), and [Xaa]_{z} is VKGR (SEQ ID NO:97) or VKGRG (SEQ ID NO:98), wherein J is 2-aminoisobutryic acid.

In certain instances of Formula (I), the sequences set forth above for [Xaa]_{w}, [Xaa]ₓ, [Xaa]_{y}, and [Xaa]_{z} can have at least one (e.g., 1, 2, 3, 4, 5, 6) amino acid substitution or deletion. In certain instances of Formula (I), there are no more than 1, 2, or 3 amino acid substitutions or deletions in [Xaa]_{w}, [Xaa]ₓ, [Xaa]_{y}, and [Xaa]_{z} combined (e.g., if there are three amino acid substitutions or deletions in [Xaa]_{w}, then there are no amino acid substitutions in any of [Xaa]ₓ, [Xaa]_{y}, and [Xaa]_{z}). The stitched GLP-1 peptides can include any amino acid sequence described herein.

The tether can include an alkyl, alkenyl, or alkynyl moiety (e.g., C₅, C₈, C₁₁, or C₁₂ alkyl, a C₅, C₈, or C₁₁ alkenyl, or C₅, C₈, C₁₁, or C₁₂ alkynyl). The tethered amino acid can be alpha disubstituted (e.g., C₁-C₃ or methyl).

In some instances, R₁ and R₄ are each independently H or C₁-C₆ alkyl. In some instances, R₁ and R₄ are each independently C₁-C₃ alkyl. In some instances, at least one of R₁ and R₄ are methyl. For example, R₁ and R₄ can both be methyl. In some instances, R₂ and R₃ are each independently alkyl (e.g., C₁₂ alkyl). In some instances, R₂ and R₃ are each independently a C₁₂ alkyl. In some instances, R₂ and R₃ are each independently a straight chain alkyl, alkenyl, or alkynyl (e.g., a straight chain C₁₂ alkyl, alkenyl, or alkynyl. In some instances, R₂ is -CH₂-CH₂-CH₂-CH₂-CH₂-CH₂-CH=CH-CH₂-CH₂-CH₂-. In some instances, R₃ is -CH₂-CH₂-CH₂-CH=CH-CH₂-CH₂-CH₂-CH₂-CH₂-CH₂-.

In another aspect, of the three alpha, alpha disubstituted stereocenters: (i) two stereocenters are in the R configuration and one stereocenter is in the S configuration; or (ii) two stereocenters are in the S configuration and one stereocenter is in the R configuration. Thus, where formula I is depicted as:

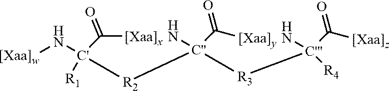

the C′ and C‴ disubstituted stereocenters can both be in the R configuration or they can both be in the S configuration. When both C′ and C‴ are in the R configuration, C″ is in the S configuration. When both C′ and C‴ are in the S configuration, C″ is in the R configuration. The double bond in each of R₂ and R₃ can be in the E or Z stereochemical configuration.

In some instances, R₃ is [R₄—K—R₄]n; and R₄ is a straight chain alkyl, alkenyl, or alkynyl.

As used herein, the term “C_{i-j,}” where i and j are integers, employed in combination with a chemical group, designates a range of the number of carbon atoms in the chemical group with i-j defining the range. For example, C₁-₆ alkyl refers to an alkyl group having 1, 2, 3, 4, 5, or 6 carbon atoms.

As used herein, the term “alkyl,” employed alone or in combination with other terms, refers to a saturated hydrocarbon group that may be straight-chain or branched. In some instances, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some instances, the alkyl group is methyl, ethyl, or propyl. The term “alkylene” refers to a linking alkyl group.

As used herein, “alkenyl,” employed alone or in combination with other terms, refers to an alkyl group having one or more carbon-carbon double bonds. In some instances, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some instances, the alkynyl moiety contains 2 to 6 or 2 to 4 carbon atoms.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some instances, the alkynyl moiety contains 2 to 6 or 2 to 4 carbon atoms.

As used herein, the term “cycloalkylalkyl,” employed alone or in combination with other terms, refers to a group of formula cycloalkyl-alkyl-. In some instances, the alkyl portion has 1 to 4, 1 to 3, 1 to 2, or 1 carbon atom(s). In some instances, the alkyl portion is methylene. In some instances, the cycloalkyl portion has 3 to 10 ring members or 3 to 7 ring members. In some instances, the cycloalkyl group is monocyclic or bicyclic. In some instances, the cycloalkyl portion is monocyclic. In some instances, the cycloalkyl portion is a C₃-₇ monocyclic cycloalkyl group.

As used herein, the term “heteroarylalkyl,” employed alone or in combination with other terms, refers to a group of formula heteroaryl-alkyl-. In some instances, the alkyl portion has 1 to 4, 1 to 3, 1 to 2, or 1 carbon atom(s). In some instances, the alkyl portion is methylene. In some instances, the heteroaryl portion is a monocyclic or bicyclic group having 1, 2, 3, or 4 heteroatoms independently selected from nitrogen, sulfur and oxygen. In some instances, the heteroaryl portion has 5 to 10 carbon atoms.

As used herein, the term “substituted” means that a hydrogen atom is replaced by a non-hydrogen group. It is to be understood that substitution at a given atom is limited by valency.

As used herein, “halo” or “halogen”, employed alone or in combination with other terms, includes fluoro, chloro, bromo, and iodo. In some instances, halo is F or C1.

In some instances, the stitched GLP-1 peptide comprising a stitched amino acid sequence of Formula (I) is a compound comprising a stitched amino acid sequence:

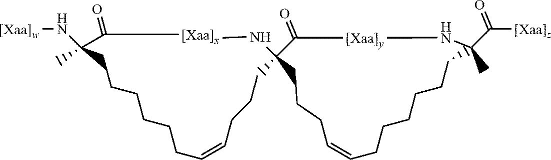

wherein:
[Xaa]_{w} is 1, 2, 3, 4, 5, 6, 7, 8, 9, or 10 amino acids;
[Xaa]_{z} is 1, 2, 3, 4, 5, 6, or 7 amino acids;
   (a) [Xaa]ₓ is SSYLEG (SEQ ID NO:46) and [Xaa]_{y} is AAKEFI (SEQ ID NO:47),
   (b) [Xaa]ₓ is SYLEGQ (SEQ ID NO:51) and [Xaa]_{y} is AKEFIA (SEQ ID NO:52);
   (c) [Xaa]ₓ is TSDVSS (SEQ ID NO:87) and [Xaa]_{y} is LEGQAA (SEQ ID NO:88),
   (d) [Xaa]ₓ is YLEGQA (SEQ ID NO:89) and [Xaa]_{y} is KEFIAW (SEQ ID NO:90),
   (e) [Xaa]ₓ is SSYLEG (SEQ ID NO:46) and [Xaa]_{y} is AAK,
   (f) [Xaa]ₓ is SYLEGQ (SEQ ID NO:51) and [Xaa]_{y} is AKE, or
   (g) [Xaa]ₓ is YLEGQA (SEQ ID NO:89) and [Xaa]_{y} is KEF, and
the amino acid sequence has at least 80% or at least 85% identity to the amino acid sequence of SEQ ID NO:3 or 4. In some instances, [Xaa]ₓ is SSYLEG (SEQ ID NO:46) and [Xaa]_{y} is AAKEFI (SEQ ID NO:47).

In some instances of Compound (1), [Xaa]_{w} is HJEGTFTSD (SEQ ID NO:45), [Xaa]ₓ is SSYLEG (SEQ ID NO:46), [Xaa]_{y} is AAKEFI (SEQ ID NO:47), and [Xaa]_{z} is WLVKGR (SEQ ID NO:48), wherein J is 2-aminoisobutyric acid. In some instances of Compound (1), [Xaa]_{w} is HJEGTFTSD (SEQ ID NO:45), [Xaa]ₓ is SSYLEG (SEQ ID NO:46), [Xaa]_{y} is AAKEFI (SEQ ID NO:47), and [Xaa]_{z} is WLVKGRG (SEQ ID NO:55), wherein J is 2-aminoisobutyric acid. In some instances of Compound (1), [Xaa]_{w} is HGEGTFTSD (SEQ ID NO:49), [Xaa]ₓ is SSYLEG (SEQ ID NO:46), [Xaa]_{y} is AAKEFI (SEQ ID NO:47), and [Xaa]_{z} is WLVKGR (SEQ ID NO:48). In some instances of Compound (1), [Xaa]_{w} is HGEGTFTSD (SEQ ID NO:49), [Xaa]ₓ is SSYLEG (SEQ ID NO:46), [Xaa]_{y} is AAKEFI (SEQ ID NO:47), and [Xaa]_{z} is WLVKGRG (SEQ ID NO:55).

In some instances of Compound (1), [Xaa]_{w} is HJEGTFTSDV (SEQ ID NO:50), [Xaa]ₓ is SYLEGQ (SEQ ID NO:51), [Xaa]_{y} is AKEFIA (SEQ ID NO:52), and [Xaa]_{z} is LVKGR (SEQ ID NO:53), wherein J is 2-aminoisobutyric acid. In some instances of Compound (1), [Xaa]_{w} is HJEGTFTSDV (SEQ ID NO:50), [Xaa]ₓ is SYLEGQ (SEQ ID NO:51), [Xaa]_{y} is AKEFIA (SEQ ID NO:52), and [Xaa]_{z} is LVKGRG (SEQ ID NO:56), wherein J is 2-aminoisobutyric acid. In some instances of Compound (1), [Xaa]_{w} is HGEGTFTSDV (SEQ ID NO:54), [Xaa]ₓ is SYLEGQ (SEQ ID NO:51), [Xaa]_{y} is AKEFIA (SEQ ID NO:52), and [Xaa]_{z} is LVKGR (SEQ ID NO:53). In some instances of Compound (1), [Xaa]_{w} is HGEGTFTSDV (SEQ ID NO:54), [Xaa]ₓ is SYLEGQ (SEQ ID NO:51), [Xaa]_{y} is AKEFIA (SEQ ID NO:52), and [Xaa]_{z} is LVKGRG (SEQ ID NO:56).

In some instances of Compound (1), [Xaa]_{z} is HGEGT (SEQ ID NO:91) or HJEGT (SEQ ID NO:92), [Xaa]ₓ is TSDVSS (SEQ ID NO:87), [Xaa]_{y} is LEGQAA (SEQ ID NO:88), and [Xaa]_{z} is EFIAWLVKGR (SEQ ID NO:93) or EFIAWLVKGRG (SEQ ID NO:94), wherein J is 2-aminoisobutryic acid.

In some instances of Compound (1), [Xaa]_{z} is HGEGTFTSDVS (SEQ ID NO:95) or HJEGTFTSDVS (SEQ ID NO:96), [Xaa]ₓ is YLEGQA (SEQ ID NO:89), [Xaa]_{y} is KEFIAW (SEQ ID NO:90), and [Xaa]_{z} is VKGR (SEQ ID NO:97) or VKGRG (SEQ ID NO:98), wherein J is 2-aminoisobutryic acid.

In certain instances of Compound (1), the sequences set forth above for [Xaa]_{w}, [Xaa]ₓ, [Xaa]_{y}, and [Xaa]_{z} can have at least one (e.g., 1, 2, 3, 4, 5, 6) amino acid substitution or deletion. In certain instances of Compound (1), there are no more than 1, 2, or 3 amino acid substitutions or deletions in [Xaa]_{w}, [Xaa]ₓ, [Xaa]_{y}, and [Xaa]_{z} combined (e.g., if there are three amino acid substitutions or deletions in [Xaa]_{w}, then there are no amino acid substitutions in any of [Xaa]ₓ, [Xaa]_{y}, and [Xaa]_{z}). The stitched GLP-1 peptides can include any amino acid sequence described herein.

The stitched peptide can be 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In a specific instance, the stitched peptide is 30, 31, 32, 33, 34, 35, 36, 37, 38, 39, 40, 41, 42, 43, 44, 45, 46, 47, 48, 49, or 50 amino acids in length. In a specific instance, the stitched peptide is 15, 16, 17, 18, 19, 20, 21, 22, 23, 24, 25, 26, 27, 28, 29, 30, 31, 32, 33, 34, or 35 amino acids in length. In a specific instance, the stitched peptide is 30 amino acids in length. In a specific instance, the stitched peptide is 31 amino acids in length. Exemplary GLP-1 peptides comprising amino acids that may be stitched are shown in FIG. 5. In one instance, the stitched peptide comprises or consists of a stitched version of the amino acid sequence set forth in SEQ ID NO:40, e.g., the product of a ring-closing metathesis reaction performed on a peptide comprising the amino acid sequence of SEQ ID NO:40.

GLP-1 stitched peptides are shown in Table 2. In one instance, the GLP-1 stitched peptide comprises or consists of the amino acid sequence set forth in SEQ ID NO:61 or 62. In one instance, the GLP-1 stitched peptide comprises or consists of the amino acid sequence set forth in SEQ ID NO:33 or 40. In one instance, the GLP-1 stitched peptide comprises the amino acid sequence set forth in SEQ ID NO:40. In one instance, the GLP-1 stitched peptide consists of the amino acid sequence set forth in SEQ ID NO:40. In one instance, the GLP-1 stitched peptide comprises or consists of the amino acid sequence set forth in SEQ ID NO:63 or 64. In one instance, the GLP-1 stitched peptide comprises or consists of the amino acid sequence set forth in SEQ ID NO:57 or 58.

In one instance, the GLP-1 stitched peptide comprises or consists of the amino acid sequence set forth in SEQ ID NO:65 or 66. In one instance, the GLP-1 stitched peptide comprises or consists of the amino acid sequence set forth in SEQ ID NO:34 or 41. In one instance, the GLP-1 stitched peptide comprises or consists of the amino acid sequence set forth in SEQ ID NO:67 or 68. In one instance, the GLP-1 stitched peptide comprises or consists of the amino acid sequence set forth in SEQ ID NO:59 or 60.

In one instance, the GLP-1 stitched peptide comprises or consists of the amino acid sequence set forth in SEQ ID NO:71 or 73. In one instance, the GLP-1 stitched peptide comprises or consists of the amino acid sequence set forth in SEQ ID NO:72 or 74. In one instance, the GLP-1 stitched peptide comprises or consists of the amino acid sequence set forth in SEQ ID NO:75 or 77. In one instance, the GLP-1 stitched peptide comprises or consists of the amino acid sequence set forth in SEQ ID NO:76 or 78.

In one instance, the GLP-1 stitched peptide comprises or consists of the amino acid sequence set forth in SEQ ID NO:79 or 81. In one instance, the GLP-1 stitched peptide comprises or consists of the amino acid sequence set forth in SEQ ID NO:80 or 82. In one instance, the GLP-1 stitched peptide comprises or consists of the amino acid sequence set forth in SEQ ID NO:83 or 85. In one instance, the GLP-1 stitched peptide comprises or consists of the amino acid sequence set forth in SEQ ID NO:84 or 86.

In certain instances, the stapled polypeptide comprises or consists of a variant of the amino acid sequence set forth in any one of SEQ ID NOs:3, 4, 31, 38, 69, and 70, wherein three amino acids each separated by 6 amino acids (i.e., positions i, i+7, and i+14) are modified to structurally-stabilize the peptide (e.g., by substituting them with non-natural amino acids to permit hydrocarbon stitching). In certain instances, the three amino acids each separated by 6 amino acids are at the amino acid positions in the GLP-1 peptide corresponding to positions 16, 23, and 30 of the amino acid sequence set forth in SEQ ID NO:2. In certain instances, the three amino acids each separated by 6 amino acids are at the amino acid position sin the GLP-1 peptide corresponding to positions 17, 24, and 31 of the amino acid sequence set forth in SEQ ID NO:2. In certain instances, the three amino acids each separated by 6 amino acids are at the amino acid position sin the GLP-1 peptide corresponding to positions 12, 19, and 26 of the amino acid sequence set forth in SEQ ID NO:2. In certain instances, the three amino acids each separated by 6 amino acids are at the amino acid position sin the GLP-1 peptide corresponding to positions 18, 25, and 32 of the amino acid sequence set forth in SEQ ID NO:2.

While hydrocarbon tethers are common, other tethers can also be employed in the stitched GLP-1 peptides described herein. For example, the tether can include one or more of an ether, thioether, ester, amine, or amide, or triazole moiety. In some cases, a naturally occurring amino acid side chain can be incorporated into the tether. For example, a tether can be coupled with a functional group such as the hydroxyl in serine, the thiol in cysteine, the primary amine in lysine, the acid in aspartate or glutamate, or the amide in asparagine or glutamine. Accordingly, it is possible to create a tether using naturally occurring amino acids rather than using a tether that is made by coupling two non-naturally occurring amino acids. It is also possible to use a single non-naturally occurring amino acid together with a naturally occurring amino acid. Triazole-containing (e.g., 1, 4 triazole or 1, 5 triazole) crosslinks can be used (see, e.g., Kawamoto et al. 2012 Journal of Medicinal Chemistry 55:1137; WO 2010/060112). In addition, other methods of performing different types of stapling are well known in the art and can be employed with the stitched GLP-1 peptides described herein (see, e.g., Lactam stapling: Shepherd et al., J. Am. Chem. Soc., 127:2974-2983 (2005); UV-cycloaddition stapling: Madden et al., Bioorg. Med. Chem. Lett., 21:1472-1475 (2011); Disulfide stapling: Jackson et al., Am. Chem. Soc., 113:9391-9392 (1991); Oxime stapling: Haney et al., Chem. Commun., 47:10915-10917 (2011); Thioether stapling: Brunel and Dawson, Chem. Commun., 552-2554 (2005); Photoswitchable stapling: J. R. Kumita et al., Proc. Natl. Acad. Sci. U. S. A., 97:3803-3808 (2000); Double-click stapling: Lau et al., Chem. Sci., 5: 1804-1809 (2014); Bis-lactam stapling: J. C. Phelan et al.,, J. Am. Chem. Soc., 119:455-460 (1997); and Bis-arylation stapling: A. M. Spokoyny et al., J. Am. Chem. Soc., 135:5946-5949 (2013)).

It is further envisioned that the length of the tether can be varied. For instance, a shorter length of tether can be used where it is desirable to provide a relatively high degree of constraint on the secondary alpha-helical structure, whereas, in some instances, it is desirable to provide less constraint on the secondary alpha-helical structure, and thus a longer tether may be desired.

In some instances, the hydrocarbon tethers (i.e., cross-links) described herein can be further manipulated. In one instance, a double bond of a hydrocarbon alkenyl tether, (e.g., as synthesized using a ruthenium-catalyzed ring closing metathesis (RCM)) can be oxidized (e.g., via epoxidation, aminohydroxylation or dihydroxylation) to provide one of compounds below.

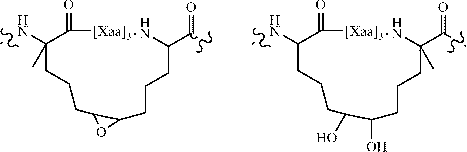

Either the epoxide moiety or one of the free hydroxyl moieties can be further functionalized. For example, the epoxide can be treated with a nucleophile, which provides additional functionality that can be used, for example, to attach a therapeutic agent. Such derivatization can alternatively be achieved by synthetic manipulation of the amino or carboxy-terminus of the polypeptide or via the amino acid side chain. Other agents can be attached to the functionalized tether, e.g., an agent that facilitates entry of the polypeptide into cells.

In some instances, alpha disubstituted amino acids are used in the polypeptide to improve the stability of the alpha helical secondary structure. However, alpha disubstituted amino acids are not required, and instances using mono-alpha substituents (e.g., in the tethered amino acids) are also envisioned.

The stitched peptides can include a drug, a toxin, a derivative of polyethylene glycol; a second polypeptide; a carbohydrate, etc. Where a polymer or other agent is linked to the stitched peptide it can be desirable for the composition to be substantially homogeneous.

The addition of polyethelene glycol (PEG) molecules can improve the pharmacokinetic and pharmacodynamic properties of the stitched peptide. For example, PEGylation can reduce renal clearance and can result in a more stable plasma concentration. PEG is a water soluble polymer and can be represented as linked to the stitched peptide as formula:

XO—(CH₂CH₂O)ₙ—CH₂CH₂—Y where n is 2 to 10,000 and X is H or a terminal modification, e.g., a C₁-₄ alkyl; and Y is an amide, carbamate or urea linkage to an amine group (including but not limited to, the epsilon amine of lysine or the N-terminus) of the stitched peptide. Y may also be a maleimide linkage to a thiol group (including but not limited to, the thiol group of cysteine). Other methods for linking PEG to a stitched peptide, directly or indirectly, are known to those of ordinary skill in the art. The PEG can be linear or branched. Various forms of PEG including various functionalized derivatives are commercially available.

\PEG having degradable linkages in the backbone can be used. For example, PEG can be prepared with ester linkages that are subject to hydrolysis. Conjugates having degradable PEG linkages are described in WO 99/34833; WO 99/14259, and U.S. 6,348,558.

In certain instances, a macromolecular polymer (e.g., PEG) is attached to a stitched peptide described herein through an intermediate linker. In certain instances, the linker is made up of from 1 to 20 amino acids linked by peptide bonds, wherein the amino acids are selected from the 20 naturally occurring amino acids. Some of these amino acids may be glycosylated, as is well understood by those in the art. In other instances, the 1 to 20 amino acids are selected from glycine, alanine, proline, asparagine, glutamine, and lysine. In other instances, a linker is made up of a majority of amino acids that are sterically unhindered, such as glycine and alanine. Non-peptide linkers are also possible. For example, alkyl linkers such as -NH(CH₂)ₙC(O)-, wherein n = 2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc. U.S. Pat. No. 5,446,090 describes a bifunctional PEG linker and its use in forming conjugates having a peptide at each of the PEG linker termini.

The stitched peptides can also be modified, e.g., to increase in vivo stability, in some instances. For example, acylating or PEGylating a structurally-stabilized (e.g., stitched) peptide facilitates increases bioavailability, increases blood circulation, alters pharmacokinetics, decreases immunogenicity and/or decreases the needed frequency of administration.

Methods of synthesizing the stitched peptides described herein are known in the art. Nevertheless, the following exemplary method may be used. It will be appreciated that the various steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T.W. Greene and P.G.M. Wuts, Protective Groups in Organic Synthesis, 3d. Ed., John Wiley and Sons (1999); L. Fieser and M. Fieser, Fieser and Fieser’s Reagents for Organic Synthesis, John Wiley and Sons (1994); and L. Paquette, ed., Encyclopedia of Reagents for Organic Synthesis, John Wiley and Sons (1995), and subsequent editions thereof.

The peptides of this invention can be made by chemical synthesis methods, which are well known to the ordinarily skilled artisan. See, for example, Fields et al., Chapter 3 in Synthetic Peptides: A User’s Guide, ed. Grant, W. H. Freeman & Co., New York, N.Y., 1992, p. 77. Hence, peptides can be synthesized using the automated Merrifield techniques of solid phase synthesis with the α-NH₂ protected by either t-Boc or Fmoc chemistry using side chain protected amino acids on, for example, an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Insertion of a stitching amino acid may be performed as described in, e.g., Young and Schultz, J Biol Chem. 2010 Apr 9; 285(15): 11039-11044. Alternatively, the longer synthetic peptides can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptides can be made in a high-throughput, combinatorial fashion, e.g., using a high-throughput multiple channel combinatorial synthesizer available from, e.g., Advanced Chemtech or Symphony X. Peptide bonds can be replaced, e.g., to increase physiological stability of the peptide, by: a retro-inverso bonds (C(O)-NH); a reduced amide bond (NH—CH₂); a thiomethylene bond (S—CH₂ or CH₂—S); an oxomethylene bond (O—CH₂ or CH₂—O); an ethylene bond (CH₂—CH₂); a thioamide bond (C(S)—NH); a trans-olefin bond (CH═CH); a fluoro substituted trans-olefin bond (CF═CH); a ketomethylene bond (C(O)—CHR) or CHR—C(O) wherein R is H or CH₃; and a fluoro-ketomethylene bond (C(O)—CFR or CFR—C(O) wherein R is H or F or CH₃.

The peptides can be further modified by: acetylation, amidation, biotinylation, cinnamoylation, farnesylation, fluoresceination, formylation, myristoylation, palmitoylation, phosphorylation (Ser, Tyr or Thr), stearoylation, succinylation and sulfurylation. As indicated above, peptides can be conjugated to, for example, polyethylene glycol (PEG); alkyl groups (e.g., C1-C20 straight or branched alkyl groups); fatty acid radicals; and combinations thereof, α, α-Disubstituted non-natural amino acids containing olefinic side chains of varying length can be synthesized by known methods (Williams et al. J. Am. Chem. Soc., 113:9276, 1991; Schafmeister et al., J. Am. Chem Soc., 122:5891, 2000; and Bird et al., Methods Enzymol., 446:369, 2008; Bird et al, Current Protocols in Chemical Biology, 2011). In some instances for peptides where an i linked to i+7, i+7 linked to i+14 stitch is used (four turns of the helix stabilized): one R-octenyl alanine (e.g., (R)-α-(7′-octenyl)alanine), one one bis-pentenyl glycine (e.g., α,α-Bis(4′-pentenyl)glycine), and one R-octenyl alanine (e.g., (R)-α-(7′-octenyl)alanine) is used. In some instances for peptides where an i linked to i+7, i+7 linked to i+14 stitch is used (four turns of the helix stabilized): one S-octenyl alanine (e.g., (S)-α-(7′-octenyl)alanine), one one bis-pentenyl glycine (e.g., α,α-Bis(4′-pentenyl)glycine), and one R-octenyl alanine (e.g., (R)-α-(7′-octenyl)alanine) is used. In some instances for peptides where an i linked to i+7, i+7 linked to i+14 stitch is used (four turns of the helix stabilized): one S-octenyl alanine (e.g., (S)-α-(7′-octenyl)alanine), one bis-pentenyl glycine (e.g., α,α-Bis(4′-pentenyl)glycine), and one S-octenyl alanine (e.g., (S)-α-(7′-octenyl)alanine) is used. In some instances for peptides where an i linked to i+7, i+7 linked to i+14 stitch is used (four turns of the helix stabilized): one R-pentenyl alanine (e.g., (R)-α-(4′-pentenyl)alanine), one bis-octenyl glycine (e.g., α,α-Bis(7′-octenyl)glycine), and one S-pentenyl alanine (e.g., (S)-α-(4′-pentenyl)alanine) is used. In some instances for peptides where an i linked to i+7, i+7 linked to i+14 stitch is used (four turns of the helix stabilized): one R-pentenyl alanine (e.g., (R)-α-(4′-pentenyl)alanine), one bis-octenyl glycine (e.g., α,α-Bis(7′-octenyl)glycine), and one R-pentenyl alanine (e.g., (R)-α-(4′-pentenyl)alanine) is used. In some instances for peptides where an i linked to i+7, i+7 linked to i+14 stitch is used (four turns of the helix stabilized): one S-pentenyl alanine (e.g., (S)-α-(4′-pentenyl)alanine), one bis-octenyl glycine (e.g., α,α-Bis(7′-octenyl)glycine), and one R-pentenyl alanine (e.g., (R)-α-(4′-pentenyl)alanine) is used. In some instances for peptides where an i linked to i+7, i+7 linked to i+14 stitch is used (four turns of the helix stabilized): one S-pentenyl alanine (e.g., (S)-α-(4′-pentenyl)alanine), one bis-octenyl glycine (e.g., α,α-Bis(7′-octenyl)glycine), and one S-pentenyl alanine (e.g., (S)-α-(4′-pentenyl)alanine) is used. R-octenyl alanine is synthesized using the same route, except that the starting chiral auxiliary confers the R-alkyl-stereoisomer. Also, 8-iodooctene is used in place of 5-iodopentene. Inhibitors are synthesized on a solid support using solid-phase peptide synthesis (SPPS) on MBHA resin (see, e.g., WO 2010/148335).

Fmoc-protected α-amino acids (other than the olefinic amino acids N-Fmoc-α,α-Bis(4′-pentenyl)glycine, (S)-N-Fmoc-α-(4′-pentenyl)alanine, (R)-N-Fmoc-α-(7′-octenyl)alanine, (R)-N-Fmoc-α-(7′-octenyl)alanine, and (R)-N-Fmoc-α-(4′-pentenyl)alanine), 2-(6-chloro-1-H-benzotriazole-1-yl)-1,1,3,3-tetramethylaminium hexafluorophosphate (HCTU), and Rink Amide MBHA are commercially available from, e.g., Novabiochem (San Diego, CA). Dimethylformamide (DMF), N-methyl-2-pyrrolidinone (NMP), N,N-diisopropylethylamine (DIEA), trifluoroacetic acid (TFA), 1,2-dichloroethane (DCE), fluorescein isothiocyanate (FITC), and piperidine are commercially available from, e.g., Sigma-Aldrich. Olefinic amino acid synthesis is reported in the art (Williams et al., Org. Synth., 80:31, 2003).

Again, methods suitable for obtaining (e.g., synthesizing), stitching, and purifying the peptides disclosed herein are also known in the art (see, e.g., Bird et. al., Methods in Enzymol., 446:369-386 (2008); Bird et al, Current Protocols in Chemical Biology, 2011; Walensky et al., Science, 305:1466-1470 (2004); Schafmeister et al., J. Am. Chem. Soc., 122:5891-5892 (2000); U.S. Pat. Application No. 12/525,123, filed Mar. 18, 2010; and U.S. Pat. No. 7,723,468, issued May 25, 2010, each of which are hereby incorporated by reference in their entirety).

In some instances, the peptides are substantially free of non-stitched peptide contaminants or are isolated. Methods for purifying peptides include, for example, synthesizing the peptide on a solid-phase support. Following cyclization, the solid-phase support may be isolated and suspended in a solution of a solvent such as DMSO, DMSO/dichloromethane mixture, or DMSO/NMP mixture. The DMSO/dichloromethane or DMSO/NMP mixture may comprise about 30%, 40%, 50% or 60% DMSO. In a specific instance, a 50%/50% DMSO/NMP solution is used. The solution may be incubated for a period of 1, 6, 12 or 24 hours, following which the resin may be washed, for example with dichloromethane or NMP. In one instance, the resin is washed with NMP. Shaking and bubbling an inert gas into the solution may be performed.

Also provided herein is a method of producing a stitched GLP-1 peptide comprising: (a) stitching a GLP-1 peptide; and (b) isolating the stitched peptide.

Properties of the stitched (cross-linked) peptides of the invention can be assayed, for example, using the methods described below and in the Examples.

Assays to Determine α-Helicity: Compounds are dissolved in an aqueous solution (e.g., 5 mM potassium phosphate solution at pH 7, or distilled H₂O, to concentrations of 25-50 µM). Circular dichroism (CD) spectra are obtained on a spectropolarimeter (e.g., Jasco J-710, Aviv) using standard measurement parameters (e.g., temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity by the reported value for a model helical decapeptide (Yang et al., Methods Enzymol. 130:208 (1986)).

Assays to Determine Melting Temperature (Tm): Cross-linked or the unmodified template peptides are dissolved in distilled H₂O or other buffer or solvent (e.g., at a final concentration of 50 µM) and Tm is determined by measuring the change in ellipticity over a temperature range (e.g., 4 to 95° C.) on a spectropolarimeter (e.g., Jasco J-710, Aviv) using standard parameters (e.g., wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

In vitro Protease Resistance Assays: A key benefit of peptide stitching is the translation of in vitro protease resistance into markedly improved pharmacokinetics in vivo. The amide bond of the peptide backbone is susceptible to hydrolysis by proteases, thereby rendering peptidic compounds vulnerable to rapid degradation in vivo. Peptide helix formation, however, typically buries and/or twists and/or shields the amide backbone and therefore may prevent or substantially retard proteolytic cleavage. The stitched peptides of the disclosure may be subjected to in vitro enzymatic proteolysis (e.g., trypsin, chymotrypsin, pepsin) to assess for any change in degradation rate compared to a corresponding uncrosslinked or alternatively stitched peptide. For example, the stitched peptide and a corresponding uncrosslinked peptide are incubated with trypsin agarose and the reactions quenched at various time points by centrifugation and subsequent HPLC injection to quantitate the residual substrate by ultraviolet absorption at 280 nm. Briefly, the stitched peptide and peptide precursor (5 mcg) are incubated with trypsin agarose (Pierce) (S/E ~125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln[S] versus time.

Stitched peptides and/or a corresponding uncrosslinked peptide can be each incubated with fresh mouse, rat and/or human serum (e.g., 1-2 mL) at 37° C. for, e.g., 0, 1, 2, 4, 8, and 24 hours. Samples of differing stitched peptide concentration may be prepared by serial dilution with serum. To determine the level of intact compound, the following procedure may be used: The samples are extracted, for example, by transferring 100 µL of sera to 2 ml centrifuge tubes followed by the addition of 10 µL of 50% formic acid and 500 µL acetonitrile and centrifugation at 14,000 RPM for 10 min at 4+/-2° C. The supernatants are then transferred to fresh 2 ml tubes and evaporated on Turbovap under N₂<10 psi, 37° C. The samples are reconstituted in 100 µL of 50:50 acetonitrile:water and submitted to LC-MS/MS analysis. Equivalent or similar procedures for testing ex vivo stability are known and may be used to determine stability of stitched peptides in serum.

In vitro Binding Assays: To assess the binding and affinity of stitched peptides and their precursors to acceptor proteins, a fluorescence polarization assay (FPA) can be used, for example. The FPA technique measures the molecular orientation and mobility using polarized light and fluorescent tracer. When excited with polarized light, fluorescent tracers (e.g., FITC) attached to molecules with high apparent molecular weights (e.g., FITC-labeled peptides bound to a large protein) emit higher levels of polarized fluorescence due to their slower rates of rotation as compared to fluorescent tracers attached to smaller molecules (e.g., FITC-labeled peptides that are free in solution).

cAMP Assay. Cyclic AMP production may be measured using, e.g., the cAMP Hunter eXpress GLP1R CHO-K1 GPCR Assay according to the manufacturer’s instructions (Eurofins, 95-0062E2CP2S). Briefly, frozen cells are thawed and plated in 96 well format for overnight incubation at 37° C. in a humidified incubator, with the top two rows of the plate reserved for the cAMP standard. To generate the standard curve, the cAMP standard is diluted to achieve an initial concentration of 2.3 µM and then serially diluted 1:3 until reaching a final dose of 39 pM. GLP-1 peptide (e.g., a stitched GLP-1 peptide) is diluted to achieve a starting concentration of 3.7 nM and serially diluted 1:3 to reach a final dose of 0.56 pM. The dilutions are then added to the plated cells and allowed to incubate at 37° C. for 30 minutes. After workup with lysis buffer and cAMP antibody incubation per the manufacturer’s protocol, luminescence is read on a SpectraMax M5 microplate reader (Molecular Devices) at equilibrium. Nonlinear regression analysis is performed using Prism software (GraphPad) to obtain EC50s for the cAMP standard curve and cAMP induction by stitched GLP-1 peptide.

Also provided herein are pharmaceutically acceptable salts of a stitched peptide described herein. In some instances, the pharmaceutically acceptable salt is an acetate, a sulfate, or a chloride. Lists of other suitable salts are found in Remington’s Pharmaceutical Sciences, 17th Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002).

### Stitched GLP-1 Peptide Variants

In some instances, internally cross-linked peptides can be made by modifying (e.g., by amino acid substitution) a peptide of any one of SEQ ID NOs:3, 4, 31, 38, 69, and 70 or a modified version thereof. In some instances, an internal stitch replaces the side chains of 3 amino acids, e.g., the stitch comprises three cross-linked amino acids, each separated by 6 amino acids (e.g., at positions i, i+ 7, and i+ 14). In some instances, the internal stitch comprises two internal staples (replacing the side chains of 3 amino acids, e.g., each staple is between two amino acids separated by 6 amino acids at positions i, i+7, and i+ 14). In some instances, the internal stitch replaces the side chain of a first amino acid and a second and a third amino acid thereby cross-linking the first amino acid (which lies between the second and third amino acids) to the second and third amino acid via an internal cross-link, wherein the first and second amino acid are separated by six amino acids, the first and the third amino acids are separated by six amino acids, and the second and third amino acids are distinct amino acids.

The stitched GLP-1 peptide comprises three modified amino acids (relative to GLP-1 peptide) joined by two internal intramolecular cross-links, thereby forming a stitch, wherein three amino acids are at positions i, i+7, and i+14. The three modified amino acids (“stitching amino acids”) can be unnatural alpha-amino acids (including, but not limited to α,α-disubstituted and N-terminal alkylated amino acids). There are many known stitching amino acids, e.g., unnatural amino acids, any of which may be included in the peptides of the present invention. Some examples of unnatural amino acids are R-octenyl alanine (“R8”, e.g., (R)-α-(7′-octenyl)alanine), S-octenyl alanine (“S8”, e.g., (S)-α-(7′-octenyl)alanine), bis-pentenyl glycine (“bis-5”, e.g., α,α-Bis(4′-pentenyl)glycine), S-pentenyl alanine (“S5”, e.g., (S)-α-(4′-pentenyl)alanine), R-pentenyl alanine (“R5”, e.g., (R)-α-(4′-pentenyl)alanine), Bis-octenyl glycine (“bis-8”, e.g., α,α-Bis(7′-octenyl)glycine), 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-l-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and /para-substituted phenylalanines (e.g., substituted with -C(=O)C₆H₅; -CF₃; -CN; -halo; –NO2; CH₃), disubstituted phenylalanines, substituted tyrosines (e.g., further substituted with -Q=O)C₆H₅; -CF₃; -CN; -halo; -NO₂; CH₃), and statine. In some instances, the amino acids forming the stitch (also referred to as the “stitching amino acids”) are R8, bis-5, S8, R5, bis-8, and S5. In some instances, the amino acids forming the stitch (also referred to as the “stitching amino acids”) are (R)-α-(7′-octenyl)alanine), α,α-Bis(4′-pentenyl)glycine, and (S)-α-(7′-octenyl)alanine, R-pentenyl alanine (e.g., (R)-α-(4′-pentenyl)alanine), bis-octenyl glycine (e.g., α,α-Bis(7′-octenyl)glycine), and S-pentenyl alanine (e.g., (S)-α-(4′-pentenyl)alanine). In some instances, the amino acids forming the stitch (also referred to as the “stitching amino acids”) are R8, bis-5, and S8. In some instances, the amino acids forming the stitch (also referred to as the “stitching amino acids”) are (R)-α-(7′-octenyl)alanine), α,α-Bis(4′-pentenyl)glycine, and (S)-α-(7′-octenyl)alanine. In some instances, the amino acids forming the stitch (also referred to as the “stitching amino acids”) are R8, bis-5, and R8, at positions i, i+7, and i+14, respectively. In some instances, the amino acids forming the stitch (also referred to as the “stitching amino acids”) are (R)-α-(7′-octenyl)alanine), α,α-Bis(4′-pentenyl)glycine, and (R)-α-(7′-octenyl)alanine. In some instances, the amino acids forming the stitch (also referred to as the “stitching amino acids”) are S8, bis-5, and R8, at positions i, i+7, and i+14, respectively. In some instances, the amino acids forming the stitch (also referred to as the “stitching amino acids”) are (S)-α-(7′-octenyl)alanine), α,α-Bis(4′-pentenyl)glycine, and (R)-α-(7′-octenyl)alanine, at positions i, i+7, and i+14, respectively. In some instances, the amino acids forming the stitch are S8, bis-5, and S8, at positions i, i+7, and i+14, respectively. In some instances, the amino acids forming the stitch (also referred to as the “stitching amino acids”) are (S)-α-(7′-octenyl)alanine), α,α-Bis(4′-pentenyl)glycine, and (S)-α-(7′-octenyl)alanine, at positions i, i+7, and i+14, respectively. In some instances, the amino acids forming the stitch (also referred to as the “stitching amino acids”) are R5, bis-8, and S5, at positions i, i+7, and i+14, respectively. In some instances, the amino acids forming the stitch (also referred to as the “stitching amino acids”) are (R)-α-(4′-pentenyl)alanine), α,α-Bis(7′-octenyl)glycine, and (S)-α-(4′-pentenyl)alanine, at positions i, i+7, and i+14, respectively. In some instances, the amino acids forming the stitch (also referred to as the “stitching amino acids”) are R5, bis-8, and R5, at positions i, i+7, and i+14, respectively. In some instances, the amino acids forming the stitch (also referred to as the “stitching amino acids”) are (R)-α-(4′-pentenyl)alanine), α,α-Bis(7′-octenyl)glycine, and (R)-α-(4′-pentenyl)alanine, at positions i, i+7, and i+14, respectively. In some instances, the amino acids forming the stitch (also referred to as the “stitching amino acids”) are S5, bis-8, and R5, at positions i, i+7, and i+14, respectively. In some instances, the amino acids forming the stitch (also referred to as the “stitching amino acids”) are (S)-α-(4′-pentenyl)alanine), α,α-Bis(7′-octenyl)glycine,and (R)-α-(4′-pentenyl)alanine, at positions i, i+7, and i+14, respectively. In some instances, the amino acids forming the stitch are S5, bis-8, and S5, at positions i, i+7, and i+14, respectively In some instances, the amino acids forming the stitch (also referred to as the “stitching amino acids”) are (S)-α-(4′-pentenyl)alanine), α,α-Bis(7′-octenyl)glycine, and (S)-α-(4′-pentenyl)alanine, at positions i, i+7, and i+14, respectively.

In some instances, stitched GLP-1 peptide variants of the disclosure are prepared from a peptide of any one of SEQ ID NOs:3, 4, 31, 38, 69, and 70 and having e.g., 1, 2, 3, 4, or 5 amino acid substitutions (e.g., 1, 2, 3, 4, or 5 amino acids are conservatively or non-conservatively substituted) and/or having, e.g., 1, 2, 3, 4, 5, 6, 7, 8, or 9 amino acid deletions from the N- and/or C-terminus (e.g., 1, 2, 3, 4, 5, 6, 7, 8, or 9 amino acids from the N- and/or C-terminus are deleted). Exemplary GLP-1 peptides that may be stitched, including variants, are provided in Table 1. For example, in certain instances, the stitched GLP-1 peptide variants of this disclosure can have 1, 2, 3, 4, or 5 amino acid substitutions in the amino acid sequence of any one of SEQ ID NOs:3, 4, 31, 38, 69, and 70 (e.g., 1, 2, 3, 4, or 5 amino acids are conservatively or non-conservatively substituted) in addition to the three modifications introducing the stitch. In some instances, one to three amino acids of the amino acid sequence of any one of SEQ ID NOs: 3, 4, 31, 38, 69, and 70 are substituted. The amino acid substitutions in the amino acid sequence of any one of SEQ ID NOs: 3, 4, 31, 38, 69, and 70 can be of amino acids that directly interact with GLP-1R or do not directly interact with GLP-1R. Examples of amino acids in the C-terminal portion of GLP-1 that directly interact with GLP-1R are Ser18, Glu21, Ala24, Ala25, Lys26, Phe28, Ile29, Leu32, and Val33 (numbered with respect to the positions in SEQ ID NO:2). Much greater variability is permitted in the amino acids in the C-terminal portion of GLP-1 that do not directly interact with GLP-1R than in the amino acids in the C-terminal portion of GLP-1 that directly interact with GLP-1R. In fact, just about every one of the amino acids in the C-terminal portion of GLP-1 that do not directly interact with GLP-1R (e.g., 5, 4, 3, 2, or 1 amino acid of the non-directly interacting amino acids) can be substituted (e.g., conservative or non-conservative amino acid substitutions or substitution with alanine) so long as the modified GLP-1 peptide retains its ability to interact with GLP-1R. In certain instances, 1, 2, or 3 amino acids in the C-terminal portion of GLP-1 that directly interact with GLP-1R are substituted with another amino acid. In some instances, the substitution(s) is/are a conservative amino acid substitution. In other instances, the substitution(s) is/are a non-conservative amino acid substitution. In some instances, where there are more than one amino acid substitutions, the substitutions are both conservative and non-conservative amino acid substitutions. In some instances, where there are more than one amino acid substitutions, each of the substitutions are conservative amino acid substitutions. In some cases, where one to three amino acids (e.g., 1, 2, or 3) of the amino acid sequence of any one of SEQ ID NOs:3, 4, 31, 38, 69, and 70 are substituted, the substitutions are all of amino acids in the C-terminal portion of GLP-1 that do not directly interact with GLP-1R. In some cases, where one to three amino acids (e.g., 1, 2, or 3) of the amino acid sequence of any one of SEQ ID NOs:3, 4, 31, 38, 69, and 70 are substituted, the substitutions are all of amino acids in the C-terminal portion of GLP-1 that directly interact with GLP-1R. In some cases, where one to three amino acids (e.g., 1, 2, or 3) of the amino acid sequence of any one of SEQ ID NOs:3, 4, 31, 38, 69, and 70 are substituted, the substitutions are both of amino acids in the C-terminal portion of GLP-1 that directly interact with GLP-1R and of amino acids in the C-terminal portion of GLP-1 that do not directly interact with GLP-1R. In certain instances, the substituted amino acid(s) are selected from the group consisting of L-Ala, D-Ala, Aib, Sar, Ser, a substituted alanine, or a substituted glycine derivative.

In certain instances, the stitched GLP-1 peptide variants of this disclosure can have 1, 2, 3, 4, or 5, amino acid removed/deleted from the C-terminus of the sequence set forth in any one of SEQ ID NOs:3, 4, 31, 38, 69, and 70. For example, in certain instances, a stitched GLP-1 peptide variant of this disclosure comprises or consists of a modified amino acid sequence of the amino acid sequence set forth in SEQ ID NO:38, wherein 1, 2, 3, 4, or 5 amino acids are removed/deleted from the C-terminus of the sequence of SEQ ID NO:38. In certain instances, the stitched GLP-1 peptide variants of this disclosure can have 1, 2, 3, 4, or 5, amino acid removed/deleted from the N-terminus of the sequence set forth in any one of SEQ ID NOs:3, 4, 31, 38, 69, and 70. In certain instances, the stitched GLP-1 peptide variants of this disclosure can have 1, 2, 3, 4, or 5, amino acid removed/deleted from both the N-terminus and C-terminus of the sequence set forth in any one of SEQ ID NOs:3, 4, 31, 38, 69, and 70. In certain instances, these removed amino acids can be replaced with 1-6 (e.g., 1, 2, 3, 4, 5, or 6) amino acids selected from the group consisting of L-Ala, D-Ala, Aib, Sar, Ser, a substituted alanine, or a substituted glycine derivative.

In certain instances, the stitched GLP-1 peptide or variant has an amino acid sequence set forth in Table 2.

The stitched GLP-1 peptide variants described herein can be optimized for therapeutic use. For example, if any of the above-described stitched GLP-1 peptide variants cause membrane disruption (cell lysis), the peptides can be optimized by lowering the overall peptide hydrophobicity. This can for example be achieved by substituting especially hydrophobic residues with an amino acid with lower hydrophobicity (e.g., alanine). Membrane disruption can also be lowered by reducing the overall positive charge of the peptide. This can be accomplished by substituting basic residues with uncharged or acidic residues. In certain instances, both the overall peptide hydrophobicity and the overall positive charge of the peptide are lowered.

In certain instances, the stitched GLP-1 peptide variants described herein are from 15 to 50 amino acids in length, from 15 to 40 amino acids in length, from 15 to 35 amino acids in length, from 15 to 30 amino acids in length, from 15 to 25 amino acids in length, from 15 to 20 amino acids in length, from 30 to 50 amino acids in length, from 30 to 40 amino acids in length, or from 30 to 35 amino acids in length. In certain instances, the stitched GLP-1 peptide variants described herein are 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In a specific instance, stitched GLP-1 peptide variants described herein are 15, 16, 17, 18, 19, 20, 21, 22, 23, 24, 25, 26, 27, 28, 29, 30, 31, 32, 33, 34, or 35 amino acids in length. In certain instances, the stitched GLP-1 peptide variants described herein are 30, 31, 32, 33, 34, 35, 36, 37, 38, 39, 40, 41, 42, 43, 44, 45, 46, 47, 48, 49, or 50 amino acids in length. In a specific instance, stitched GLP-1 peptide variants described herein are 30 amino acids in length. In a specific instance, stitched GLP-1 peptide variants described herein are 31 amino acids in length.

In certain instances, the stitched GLP-1 peptide variant comprises or consists of the amino acid sequence set forth in Table 2.

Also provided herein are pharmaceutically acceptable salts of a stitched peptide variant described herein. In some instances, the pharmaceutically acceptable salt is an acetate, a sulfate, or a chloride. Lists of other suitable salts are found in Remington’s Pharmaceutical Sciences, 17th Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002).

### Exemplary Stitched GLP-1 Peptide

In a specific instance, the stitched GLP-1 peptide is based on the amino acid sequence of SEQ ID NO:40 with 0 to 6 (i.e., 0, 1, 2, 3, 4, 5, 6) amino acid substitutions, insertions, and/or deletions. In a specific instance, the stitched GLP-1 peptide is based on the amino acid sequence of SEQ ID NO:40 with 0 to 6 (i.e., 0, 1, 2, 3, 4, 5, 6) amino acid substitutions. In a specific instance, the stitched GLP-1 peptide is based on the amino acid sequence of SEQ ID NO:40 with 0 to 3 (i.e., 0, 1, 2, 3) amino acid deletions from the N-terminus. In a specific instance, the stitched GLP-1 peptide is based on the amino acid sequence of SEQ ID NO:40 with 0 to 3 (i.e., 0, 1, 2, 3) amino acid deletions from the C-terminus. In a particular instance, the stitched peptide further comprises one or more of the modifications described in the sections “GLP-1 Peptides” and “Stitched Peptides” above. In certain instances, the 1 to 6 (i.e., 0, 1, 2, 3, 4, 5, 6) amino acids of SEQ ID NO:40 that are substituted by another amino acid comprise a substitution at position 2 of the amino acid sequence set forth in SEQ ID NO:40. In certain instances, the 1 to 6 (i.e., 0, 1, 2, 3, 4, 5, 6) amino acids of the amino acid sequence set forth in SEQ ID NO:40 that are substituted by another amino acid are on the GLP-1R-non-interacting face of the C-terminal portion of the GLP-1 helix of the amino acid sequence set forth in SEQ ID NO:40. Examples of amino acids in the C-terminal portion of GLP-1 that directly interact with GLP-1R are Ser18, Glu21, Ala24, Ala25, Lys26, Phe28, Ile29, Leu32, and Val33 (numbered with respect to the positions in SEQ ID NO:2). In some instances, the 1 to 3 amino acids in the amino acid sequence set forth in SEQ ID NO:40 that are removed from the N-terminus or are removed and replaced with 1 to 6 amino acids from the group consisting of alanine, D-alanine, α-aminoisobutyric acid, N-methyl glycine, serine, a substituted alanine, and a glycine derivative. In some instances, the 1 to 3 amino acids in the amino acid sequence set forth in SEQ ID NO:40 that are removed from the C-terminus or are removed and replaced with 1 to 6 amino acids from the group consisting of alanine, D-alanine, α-aminoisobutyric acid, N-methyl glycine, serine, a substituted alanine, and a glycine derivative. In some instances, 0 to 6 amino acids on the GLP-1R-non-interacting face of the GLP-1 C-terminal portion of the amino acid sequence set forth in SEQ ID NO:40 are substituted with an amino acid selected from the group consisting of alanine, D-alanine, α-aminoisobutyric acid, N-methyl glycine, serine, a substituted alanine, and a glycine derivative. In certain instances, the 1 to 6 amino acids of the amino acid sequence set forth in SEQ ID NO:40 that are substituted by another amino acid are on the GLP-1R-interacting face of the C-terminal portion of the GLP-1 helix of the amino acid sequence set forth in SEQ ID NO:40. In other instances, the 1 to 6 amino acids of the amino acid sequence set forth in SEQ ID NO:40 that are substituted by another amino acid are on the GLP-1R-non-interacting face of the C-terminal portion and the GLP-1R-interacting face of the C-terminal portion of the GLP-1 helix of the amino acid sequence set forth in SEQ ID NO:40. In certain instances, the 1 to 6 amino acids of the amino acid sequence set forth in SEQ ID NO:40 are substituted by an amino acid or amino acids selected from the group consisting of L-alanine, D-alanine, α-aminoisobutyric acid, N-methyl glycine, serine, a substituted alanine, and a glycine derivative.

In certain instances, the stitched GLP-1 peptide comprises a stitched GLP-1 peptide comprising or consisting of Compound (1), wherein [Xaa]_{w} is HJEGTFTSD (SEQ ID NO:45), [Xaa]ₓ is SSYLEG (SEQ ID NO:46), [Xaa]_{y} is AAKEFI (SEQ ID NO:47), and [Xaa]_{z} is WLVKGR (SEQ ID NO:48), wherein J is 2-aminoisobutyric acid.

In another instance, the stitched GLP-1 peptide comprises a stitched (internally cross-linked) form of the amino acid sequence of SEQ ID NO:40 (e.g., the product of a ring-closing metathesis reaction on the amino acid sequence set forth in SEQ ID NO:40). In another instance, the stitched GLP-1 peptide consists of a stitched (internally cross-linked) form of the amino acid sequence of SEQ ID NO:40 (e.g., the product of a ring-closing metathesis reaction on the amino acid sequence set forth in SEQ ID NO:40).

In a specific instance, the stitched GLP-1 peptide is based on the amino acid sequence of SEQ ID NO:58 with 0 to 6 (i.e., 0, 1, 2, 3, 4, 5, 6) amino acid substitutions, insertions, and/or deletions. In a specific instance, the stitched GLP-1 peptide is based on the amino acid sequence of SEQ ID NO:58 with 0 to 6 (i.e., 0, 1, 2, 3, 4, 5, 6) amino acid substitutions. In a specific instance, the stitched GLP-1 peptide is based on the amino acid sequence of SEQ ID NO:58 with 0 to 3 (i.e., 0, 1, 2, 3) amino acid deletions from the N-terminus. In a specific instance, the stitched GLP-1 peptide is based on the amino acid sequence of SEQ ID NO:58 with 0 to 3 (i.e., 0, 1, 2, 3) amino acid deletions from the C-terminus. In a particular instance, the stitched peptide further comprises one or more of the modifications described in the sections “GLP-1 Peptides” and “Stitched Peptides” above. In certain instances, the 1 to 6 (i.e., 0, 1, 2, 3, 4, 5, 6) amino acids of the amino acid sequence set forth in SEQ ID NO:58 that are substituted by another amino acid comprise a substitution at position 2 of the amino acid sequence set forth in SEQ ID NO:58. In certain instances, the 1 to 6 (i.e., 0, 1, 2, 3, 4, 5, 6) amino acids of the amino acid sequence set forth in SEQ ID NO:58 that are substituted by another amino acid are on the GLP-1R-non-interacting face of the C-terminal portion of GLP-1 helix of the amino acid sequence set forth in SEQ ID NO:58. Examples of amino acids in the C-terminal portion of GLP-1 that directly interact with GLP-1R are Ser18, Glu21, Ala24, Ala25, Lys26, Phe28, Ile29, Leu32, and Val33 (numbered with respect to the positions in the amino acid sequence set forth in SEQ ID NO:2). In some instances, the 1 to 3 amino acids in the amino acid sequence set forth in SEQ ID NO:58 that are removed from the N-terminus or are removed and replaced with 1 to 6 amino acids from the group consisting of alanine, D-alanine, α-aminoisobutyric acid, N-methyl glycine, serine, a substituted alanine, and a glycine derivative. In some instances, the 1 to 3 amino acids in the amino acid sequence set forth in SEQ ID NO:58 that are removed from the C-terminus or are removed and replaced with 1 to 6 amino acids from the group consisting of alanine, D-alanine, α-aminoisobutyric acid, N-methyl glycine, serine, a substituted alanine, and a glycine derivative. In some instances, 0 to 6 amino acids on the GLP-1R-non-interacting face of the C-terminal portion of the GLP-1 amino acid sequence set forth in SEQ ID NO:58 are substituted with an amino acid selected from the group consisting of alanine, D-alanine, α-aminoisobutyric acid, N-methyl glycine, serine, a substituted alanine, and a glycine derivative. In certain instances, the 1 to 6 amino acids of the amino acid sequence set forth in SEQ ID NO:58 that are substituted by another amino acid are on the GLP-1R-interacting face of the C-terminal portion of the GLP-1 helix of the amino acid sequence set forth in SEQ ID NO:58. In other instances, the 1 to 6 amino acids of the amino acid sequence set forth in SEQ ID NO:58 that are substituted by another amino acid are on the GLP-1R-non-interacting face of the C-terminal portion and the GLP-1R-interacting face of the C-terminal portion of the GLP-1 helix of the amino acid sequence set forth in SEQ ID NO:58. In certain instances, the 1 to 6 amino acids of the amino acid sequence set forth in SEQ ID NO:58 are substituted by an amino acid or amino acids selected from the group consisting of L-alanine, D-alanine, α-aminoisobutyric acid, N-methyl glycine, serine, a substituted alanine, and a glycine derivative.

In certain instances, the stitched GLP-1 peptide comprises a stitched GLP-1 peptide comprising or consisting of Compound (1), wherein [Xaa]_{w} is HJEGTFTSD (SEQ ID NO:45), [Xaa]ₓ is SSYLEG (SEQ ID NO:46), [Xaa]_{y} is AAKEFI (SEQ ID NO:47), and [Xaa]_{z} is WLVKGRG (SEQ ID NO:55), wherein J is 2-aminoisobutyric acid.

In another instance, the stitched GLP-1 peptide comprises a stitched (internally cross-linked) form of the amino acid sequence of SEQ ID NO:58 (e.g., the product of a ring-closing metathesis reaction on the amino acid sequence set forth in SEQ ID NO:58). In another instance, the stitched GLP-1 peptide consists of a stitched (internally cross-linked) form of the amino acid sequence of SEQ ID NO:58 (e.g., the product of a ring-closing metathesis reaction on the amino acid sequence set forth in SEQ ID NO:58).

In a specific instance, the stitched GLP-1 peptide is based on the amino acid sequence of SEQ ID NO:33 with 0 to 6 (i.e., 0, 1, 2, 3, 4, 5, 6) amino acid substitutions, insertions, and/or deletions. In a specific instance, the stitched GLP-1 peptide is based on the amino acid sequence of SEQ ID NO:33 with 0 to 6 (i.e., 0, 1, 2, 3, 4, 5, 6) amino acid substitutions. In a specific instance, the stitched GLP-1 peptide is based on the amino acid sequence of SEQ ID NO:33 with 0 to 3 (i.e., 0, 1, 2, 3) amino acid deletions from the N-terminus. In a specific instance, the stitched GLP-1 peptide is based on the amino acid sequence of SEQ ID NO:33 with 0 to 3 (i.e., 0, 1, 2, 3) amino acid deletions from the C-terminus. In a particular instance, the stitched peptide further comprises one or more of the modifications described in the sections “GLP-1 Peptides” and “Stitched Peptides” above. In certain instances, the 1 to 6 (i.e., 0, 1, 2, 3, 4, 5, 6) amino acids of the amino acid sequence set forth in SEQ ID NO:33 that are substituted by another amino acid comprise a substitution at position 2 of the amino acid sequence set forth in SEQ ID NO:33. In certain instances, the 1 to 6 (i.e., 0, 1, 2, 3, 4, 5, 6) amino acids of the amino acid sequence set forth in SEQ ID NO:33 that are substituted by another amino acid are on the GLP-1R-non-interacting face of the C-terminal portion of the GLP-1 helix of the amino acid sequence set forth in SEQ ID NO:33. Examples of amino acids in the C-terminal portion of GLP-1 that directly interact with GLP-1R are Ser18, Glu21, Ala24, Ala25, Lys26, Phe28, Ile29, Leu32, and Val33 (numbered with respect to the positions in the amino acid sequence set forth in SEQ ID NO:2). In some instances, the 1 to 3 amino acids in the amino acid sequence set forth in SEQ ID NO:33 that are removed from the N-terminus or are removed and replaced with 1 to 6 amino acids from the group consisting of alanine, D-alanine, α-aminoisobutyric acid, N-methyl glycine, serine, a substituted alanine, and a glycine derivative. In some instances, the 1 to 3 amino acids in the amino acid sequence set forth in SEQ ID NO:33 that are removed from the C-terminus or are removed and replaced with 1 to 6 amino acids from the group consisting of alanine, D-alanine, α-aminoisobutyric acid, N-methyl glycine, serine, a substituted alanine, and a glycine derivative. In some instances, 0 to 6 amino acids on the GLP-1R-non-interacting face of the C-terminal portion of the amino acid sequence set forth in SEQ ID NO:33 are substituted with an amino acid selected from the group consisting of alanine, D-alanine, α-aminoisobutyric acid, N-methyl glycine, serine, a substituted alanine, and a glycine derivative. In certain instances, the 1 to 6 amino acids of the amino acid sequence set forth in SEQ ID NO:33 that are substituted by another amino acid are on the GLP-1R-interacting face of the C-terminal portion of the GLP-1 helix of the amino acid sequence set forth in SEQ ID NO:33. In other instances, the 1 to 6 amino acids of the amino acid sequence set forth in SEQ ID NO:33 that are substituted by another amino acid are on the GLP-1R-non-interacting face of the C-terminal portion and the GLP-1R-interacting face of the C-terminal portion of the GLP-1 helix of the amino acid sequence set forth in SEQ ID NO:33. In certain instances, the 1 to 6 amino acids of the amino acid sequence set forth in SEQ ID NO:33 are substituted by an amino acid or amino acids selected from the group consisting of L-alanine, D-alanine, α-aminoisobutyric acid, N-methyl glycine, serine, a substituted alanine, and a glycine derivative.

In certain instances, the stitched GLP-1 peptide comprises a stitched GLP-1 peptide comprising or consisting of Compound (1), wherein [Xaa]_{w} is HGEGTFTSD (SEQ ID NO:49), [Xaa]ₓ is SSYLEG (SEQ ID NO:46), [Xaa]_{y} is AAKEFI (SEQ ID NO:47), and [Xaa]_{z} is WLVKGR (SEQ ID NO:48).

In another instance, the stitched GLP-1 peptide comprises a stitched (internally cross-linked) form of the amino acid sequence of SEQ ID NO:33 (e.g., the product of a ring-closing metathesis reaction on the amino acid sequence set forth in SEQ ID NO:33). In another instance, the stitched GLP-1 peptide consists of a stitched (internally cross-linked) form of the amino acid sequence of SEQ ID NO:33 (e.g., the product of a ring-closing metathesis reaction on the amino acid sequence set forth in SEQ ID NO:33).

In a specific instance, the stitched GLP-1 peptide is based on the amino acid sequence of SEQ ID NO:57 with 0 to 6 (i.e., 0, 1, 2, 3, 4, 5, 6) amino acid substitutions, insertions, and/or deletions. In a specific instance, the stitched GLP-1 peptide is based on the amino acid sequence of SEQ ID NO:57 with 0 to 6 (i.e., 0, 1, 2, 3, 4, 5, 6) amino acid substitutions. In a specific instance, the stitched GLP-1 peptide is based on the amino acid sequence of SEQ ID NO:57 with 0 to 3 (i.e., 0, 1, 2, 3) amino acid deletions from the N-terminus. In a specific instance, the stitched GLP-1 peptide is based on the amino acid sequence of SEQ ID NO:57 with 0 to 3 (i.e., 0, 1, 2, 3) amino acid deletions from the C-terminus. In a particular instance, the stitched peptide further comprises one or more of the modifications described in the sections “GLP-1 Peptides” and “Stitched Peptides” above. In certain instances, the 1 to 6 (i.e., 0, 1, 2, 3, 4, 5, 6) amino acids of the amino acid sequence set forth in SEQ ID NO:57 that are substituted by another amino acid comprise a substitution at position 2 of the amino acid sequence set forth in SEQ ID NO:57. In certain instances, the 1 to 6 (i.e., 0, 1, 2, 3, 4, 5, 6) amino acids of the amino acid sequence set forth in SEQ ID NO:57 that are substituted by another amino acid are on the GLP-1R-non-interacting face of the C-terminal portion of the GLP-1 helix of the amino acid sequence set forth in SEQ ID NO:57. Examples of amino acids in the C-terminal portion of GLP-1 that directly interact with GLP-1R are Ser18, Glu21, Ala24, Ala25, Lys26, Phe28, Ile29, Leu32, and Val33 (numbered with respect to the positions in the amino acid sequence set forth in SEQ ID NO:2). In some instances, the 1 to 3 amino acids in the amino acid sequence set forth in SEQ ID NO:57 that are removed from the N-terminus or are removed and replaced with 1 to 6 amino acids from the group consisting of alanine, D-alanine, α-aminoisobutyric acid, N-methyl glycine, serine, a substituted alanine, and a glycine derivative. In some instances, the 1 to 3 amino acids in the amino acid sequence set forth in SEQ ID NO:57 that are removed from the C-terminus or are removed and replaced with 1 to 6 amino acids from the group consisting of alanine, D-alanine, α-aminoisobutyric acid, N-methyl glycine, serine, a substituted alanine, and a glycine derivative. In some instances, 0 to 6 amino acids on the GLP-1R-non-interacting face of the C-terminal portion of the amino acid sequence set forth in SEQ ID NO:57 are substituted with an amino acid selected from the group consisting of alanine, D-alanine, α-aminoisobutyric acid, N-methyl glycine, serine, a substituted alanine, and a glycine derivative. In certain instances, the 1 to 6 amino acids of the amino acid sequence set forth in SEQ ID NO:57 that are substituted by another amino acid are on the GLP-1R-interacting face of the C-terminal portion of the GLP-1 helix of the amino acid sequence set forth in SEQ ID NO:57. In other instances, the 1 to 6 amino acids of the amino acid sequence set forth in SEQ ID NO:57 that are substituted by another amino acid are on the GLP-1R-non-interacting face of the C-terminal portion and the GLP-1R-interacting face of the C-terminal portion of the GLP-1 helix of the amino acid sequence set forth in SEQ ID NO:57. In certain instances, the 1 to 6 amino acids of the amino acid sequence set forth in SEQ ID NO:57 are substituted by an amino acid or amino acids selected from the group consisting of L-alanine, D-alanine, α-aminoisobutyric acid, N-methyl glycine, serine, a substituted alanine, and a glycine derivative.

In certain instances, the stitched GLP-1 peptide comprises a stitched GLP-1 peptide comprising or consisting of Compound (1), wherein [Xaa]_{w} is HGEGTFTSD (SEQ ID NO:49), [Xaa]ₓ is SSYLEG (SEQ ID NO:46), [Xaa]_{y} is AAKEFI (SEQ ID NO:47), and [Xaa]_{z} is WLVKGRG (SEQ ID NO:55).

In another instance, the stitched GLP-1 peptide comprises a stitched (internally cross-linked) form of the amino acid sequence of SEQ ID NO:57 (e.g., the product of a ring-closing metathesis reaction on the amino acid sequence set forth in SEQ ID NO:57). In another instance, the stitched GLP-1 peptide consists of a stitched (internally cross-linked) form of the amino acid sequence of SEQ ID NO:57 (e.g., the product of a ring-closing metathesis reaction on the amino acid sequence set forth in SEQ ID NO:57).

In a specific instance, the stitched GLP-1 peptide is based on the amino acid sequence of SEQ ID NO:41 with 0 to 6 (i.e., 0, 1, 2, 3, 4, 5, 6) amino acid substitutions, insertions, and/or deletions. In a specific instance, the stitched GLP-1 peptide is based on the amino acid sequence of SEQ ID NO:41 with 0 to 6 (i.e., 0, 1, 2, 3, 4, 5, 6) amino acid substitutions. In a specific instance, the stitched GLP-1 peptide is based on the amino acid sequence of SEQ ID NO:41 with 0 to 3 (i.e., 0, 1, 2, 3) amino acid deletions from the N-terminus. In a specific instance, the stitched GLP-1 peptide is based on the amino acid sequence of SEQ ID NO:41 with 0 to 3 (i.e., 0, 1, 2, 3) amino acid deletions from the C-terminus. In a particular instance, the stitched peptide further comprises one or more of the modifications described in the sections “GLP-1 Peptides” and “Stitched Peptides” above. In certain instances, the 1 to 6 (i.e., 0, 1, 2, 3, 4, 5, 6) amino acids of the amino acid sequence set forth in SEQ ID NO:41 that are substituted by another amino acid comprise a substitution at position 2 of the amino acid sequence set forth in SEQ ID NO:41. In certain instances, the 1 to 6 (i.e., 0, 1, 2, 3, 4, 5, 6) amino acids of the amino acid sequence set forth in SEQ ID NO:41 that are substituted by another amino acid are on the GLP-1R-non-interacting face of the C-terminal portion of the GLP-1 helix of the amino acid sequence set forth in SEQ ID NO:41. Examples of amino acids in the C-terminal portion of GLP-1 that directly interact with GLP-1R are Ser18, Glu21, Ala24, Ala25, Lys26, Phe28, Ile29, Leu32, and Val33 (numbered with respect to the positions in the amino acid sequence set forth in SEQ ID NO:2). In some instances, the 1 to 3 amino acids in the amino acid sequence set forth in SEQ ID NO:41 that are removed from the N-terminus or are removed and replaced with 1 to 6 amino acids from the group consisting of alanine, D-alanine, α-aminoisobutyric acid, N-methyl glycine, serine, a substituted alanine, and a glycine derivative. In some instances, the 1 to 3 amino acids in the amino acid sequence set forth in SEQ ID NO:41 that are removed from the C-terminus or are removed and replaced with 1 to 6 amino acids from the group consisting of alanine, D-alanine, α-aminoisobutyric acid, N-methyl glycine, serine, a substituted alanine, and a glycine derivative. In some instances, 0 to 6 amino acids on the GLP-1R-non-interacting face of the C-terminal portion of the amino acid sequence set forth in SEQ ID NO:41 are substituted with an amino acid selected from the group consisting of alanine, D-alanine, α-aminoisobutyric acid, N-methyl glycine, serine, a substituted alanine, and a glycine derivative. In certain instances, the 1 to 6 amino acids of the amino acid sequence set forth in SEQ ID NO:41 that are substituted by another amino acid are on the GLP-1R-interacting face of the C-terminal portion of the GLP-1 helix of the amino acid sequence set forth in SEQ ID NO:41. In other instances, the 1 to 6 amino acids of the amino acid sequence set forth in SEQ ID NO:41 that are substituted by another amino acid are on the GLP-1R-non-interacting face of the C-terminal portion and the GLP-1R-interacting face of the C-terminal portion of the GLP-1 helix of the amino acid sequence set forth in SEQ ID NO:41. In certain instances, the 1 to 6 amino acids of the amino acid sequence set forth in SEQ ID NO:41 are substituted by an amino acid or amino acids selected from the group consisting of L-alanine, D-alanine, α-aminoisobutyric acid, N-methyl glycine, serine, a substituted alanine, and a glycine derivative.

In certain instances, the stitched GLP-1 peptide comprises a stitched GLP-1 peptide comprising or consisting of Compound (1), wherein [Xaa]_{w} is HJEGTFTSDV (SEQ ID NO:50), [Xaa]ₓ is SYLEGQ (SEQ ID NO:51), [Xaa]_{y} is AKEFIA (SEQ ID NO:52), and [Xaa]_{z} is LVKGR (SEQ ID NO:53), wherein J is 2-aminoisobutyric acid.

In another instance, the stitched GLP-1 peptide comprises a stitched (internally cross-linked) form of the amino acid sequence of SEQ ID NO:41 (e.g., the product of a ring-closing metathesis reaction on the amino acid sequence set forth in SEQ ID NO:41). In another instance, the stitched GLP-1 peptide consists of a stitched (internally cross-linked) form of the amino acid sequence of SEQ ID NO:41 (e.g., the product of a ring-closing metathesis reaction on the amino acid sequence set forth in SEQ ID NO:41).

In a specific instance, the stitched GLP-1 peptide is based on the amino acid sequence of SEQ ID NO:60 with 0 to 6 (i.e., 0, 1, 2, 3, 4, 5, 6) amino acid substitutions, insertions, and/or deletions. In a specific instance, the stitched GLP-1 peptide is based on the amino acid sequence of SEQ ID NO:60 with 0 to 6 (i.e., 0, 1, 2, 3, 4, 5, 6) amino acid substitutions. In a specific instance, the stitched GLP-1 peptide is based on the amino acid sequence of SEQ ID NO:60 with 0 to 3 (i.e., 0, 1, 2, 3) amino acid deletions from the N-terminus. In a specific instance, the stitched GLP-1 peptide is based on the amino acid sequence of SEQ ID NO:60 with 0 to 3 (i.e., 0, 1, 2, 3) amino acid deletions from the C-terminus. In a particular instance, the stitched peptide further comprises one or more of the modifications described in the sections “GLP-1 Peptides” and “Stitched Peptides” above. In certain instances, the 1 to 6 (i.e., 0, 1, 2, 3, 4, 5, 6) amino acids of the amino acid sequence set forth in SEQ ID NO:60 that are substituted by another amino acid comprise a substitution at position 2 of the amino acid sequence set forth in SEQ ID NO:60. In certain instances, the 1 to 6 (i.e., 0, 1, 2, 3, 4, 5, 6) amino acids of the amino acid sequence set forth in SEQ ID NO:60 that are substituted by another amino acid are on the GLP-1R-non-interacting face of the C-terminal portion of the GLP-1 helix of the amino acid sequence set forth in SEQ ID NO:60. Examples of amino acids in the C-terminal portion of GLP-1 that directly interact with GLP-1R are Ser18, Glu21, Ala24, Ala25, Lys26, Phe28, Ile29, Leu32, and Val33 (numbered with respect to the positions in the amino acid sequence set forth in SEQ ID NO:2). In some instances, the 1 to 3 amino acids in the amino acid sequence set forth in SEQ ID NO:60 that are removed from the N-terminus or are removed and replaced with 1 to 6 amino acids from the group consisting of alanine, D-alanine, α-aminoisobutyric acid, N-methyl glycine, serine, a substituted alanine, and a glycine derivative. In some instances, the 1 to 3 amino acids in the amino acid sequence set forth in SEQ ID NO:60 that are removed from the C-terminus or are removed and replaced with 1 to 6 amino acids from the group consisting of alanine, D-alanine, α-aminoisobutyric acid, N-methyl glycine, serine, a substituted alanine, and a glycine derivative. In some instances, 0 to 6 amino acids on the GLP-1R-non-interacting face of the C-terminal portion of the amino acid sequence set forth in SEQ ID NO:60 are substituted with an amino acid selected from the group consisting of alanine, D-alanine, α-aminoisobutyric acid, N-methyl glycine, serine, a substituted alanine, and a glycine derivative. In certain instances, the 1 to 6 amino acids of the amino acid sequence set forth in SEQ ID NO:60 that are substituted by another amino acid are on the GLP-1R-interacting face of the C-terminal portion of the GLP-1 helix of the amino acid sequence set forth in SEQ ID NO:60. In other instances, the 1 to 6 amino acids of the amino acid sequence set forth in SEQ ID NO:60 that are substituted by another amino acid are on the GLP-1R-non-interacting face of the C-terminal portion and the GLP-1R-interacting face of the C-terminal portion of the GLP-1 helix of the amino acid sequence set forth in SEQ ID NO:60. In certain instances, the 1 to 6 amino acids of the amino acid sequence set forth in SEQ ID NO:60 are substituted by an amino acid or amino acids selected from the group consisting of L-alanine, D-alanine, α-aminoisobutyric acid, N-methyl glycine, serine, a substituted alanine, and a glycine derivative.In certain instances, the stitched GLP-1 peptide comprises a stitched GLP-1 peptide comprising or consisting of Compound (1), wherein [Xaa]_{w} is HJEGTFTSDV (SEQ ID NO:50), [Xaa]ₓ is SYLEGQ (SEQ ID NO:51), [Xaa]_{y} is AKEFIA (SEQ ID NO:52), and [Xaa]_{z} is LVKGRG (SEQ ID NO:56), wherein J is 2-aminoisobutyric acid.

In another instance, the stitched GLP-1 peptide comprises a stitched (internally cross-linked) form of the amino acid sequence of SEQ ID NO:60 (e.g., the product of a ring-closing metathesis reaction on the amino acid sequence set forth in SEQ ID NO:60). In another instance, the stitched GLP-1 peptide consists of a stitched (internally cross-linked) form of the amino acid sequence of SEQ ID NO:60 (e.g., the product of a ring-closing metathesis reaction on the amino acid sequence set forth in SEQ ID NO:60).

In a specific instance, the stitched GLP-1 peptide is based on the amino acid sequence of SEQ ID NO:34 with 0 to 6 (i.e., 0, 1, 2, 3, 4, 5, 6) amino acid substitutions, insertions, and/or deletions. In a specific instance, the stitched GLP-1 peptide is based on the amino acid sequence of SEQ ID NO:34 with 0 to 6 (i.e., 0, 1, 2, 3, 4, 5, 6) amino acid substitutions. In a specific instance, the stitched GLP-1 peptide is based on the amino acid sequence of SEQ ID NO:34 with 0 to 3 (i.e., 0, 1, 2, 3) amino acid deletions from the N-terminus. In a specific instance, the stitched GLP-1 peptide is based on the amino acid sequence of SEQ ID NO:34 with 0 to 3 (i.e., 0, 1, 2, 3) amino acid deletions from the C-terminus. In a particular instance, the stitched peptide further comprises one or more of the modifications described in the sections “GLP-1 Peptides” and “Stitched Peptides” above. In certain instances, the 1 to 6 (i.e., 0, 1, 2, 3, 4, 5, 6) amino acids of the amino acid sequence set forth in SEQ ID NO:34 that are substituted by another amino acid comprise a substitution at position 2 of the amino acid sequence set forth in SEQ ID NO:34. In certain instances, the 1 to 6 (i.e., 0, 1, 2, 3, 4, 5, 6) amino acids of the amino acid sequence set forth in SEQ ID NO:34 that are substituted by another amino acid are on the GLP-1R-non-interacting face of the C-terminal portion of the GLP-1 helix of the amino acid sequence set forth in SEQ ID NO:34. Examples of amino acids in the C-terminal portion of GLP-1 that directly interact with GLP-1R are Ser18, Glu21, Ala24, Ala25, Lys26, Phe28, Ile29, Leu32, and Val33 (numbered with respect to the positions in the amino acid sequence set forth in SEQ ID NO:2). In some instances, the 1 to 3 amino acids in the amino acid sequence set forth in SEQ ID NO:34 that are removed from the N-terminus or are removed and replaced with 1 to 6 amino acids from the group consisting of alanine, D-alanine, α-aminoisobutyric acid, N-methyl glycine, serine, a substituted alanine, and a glycine derivative. In some instances, the 1 to 3 amino acids in the amino acid sequence set forth in SEQ ID NO:34 that are removed from the C-terminus or are removed and replaced with 1 to 6 amino acids from the group consisting of alanine, D-alanine, α-aminoisobutyric acid, N-methyl glycine, serine, a substituted alanine, and a glycine derivative. In some instances, 0 to 6 amino acids on the GLP-1R-non-interacting face of the C-terminal portion of the amino acid sequence set forth in SEQ ID NO:34 are substituted with an amino acid selected from the group consisting of alanine, D-alanine, α-aminoisobutyric acid, N-methyl glycine, serine, a substituted alanine, and a glycine derivative. In certain instances, the 1 to 6 amino acids of the amino acid sequence set forth in SEQ ID NO:34 that are substituted by another amino acid are on the GLP-1R-interacting face of the C-terminal portion of the GLP-1 helix of the amino acid sequence set forth in SEQ ID NO:34. In other instances, the 1 to 6 amino acids of the amino acid sequence set forth in SEQ ID NO:34 that are substituted by another amino acid are on the GLP-1R-non-interacting face of the C-terminal portion and the GLP-1R-interacting face of the C-terminal portion of the GLP-1 helix of the amino acid sequence set forth in SEQ ID NO:34. In certain instances, the 1 to 6 amino acids of the amino acid sequence set forth in SEQ ID NO:34 are substituted by an amino acid or amino acids selected from the group consisting of L-alanine, D-alanine, α-aminoisobutyric acid, N-methyl glycine, serine, a substituted alanine, and a glycine derivative.

In certain instances, the stitched GLP-1 peptide comprises a stitched GLP-1 peptide comprising or consisting of Compound (1), wherein [Xaa]_{w} is HGEGTFTSDV (SEQ ID NO:54), [Xaa]ₓ is SYLEGQ (SEQ ID NO:51), [Xaa]_{y} is AKEFIA (SEQ ID NO:52), and [Xaa]_{z} is LVKGR (SEQ ID NO:53).

In another instance, the stitched GLP-1 peptide comprises a stitched (internally cross-linked) form of the amino acid sequence of SEQ ID NO:34 (e.g., the product of a ring-closing metathesis reaction on the amino acid sequence set forth in SEQ ID NO:34). In another instance, the stitched GLP-1 peptide consists of a stitched (internally cross-linked) form of the amino acid sequence of SEQ ID NO:34 (e.g., the product of a ring-closing metathesis reaction on the amino acid sequence set forth in SEQ ID NO:34).

In a specific instance, the stitched GLP-1 peptide is based on the amino acid sequence of SEQ ID NO:59 with 0 to 6 (i.e., 0, 1, 2, 3, 4, 5, 6) amino acid substitutions, insertions, and/or deletions. In a specific instance, the stitched GLP-1 peptide is based on the amino acid sequence of SEQ ID NO:59 with 0 to 6 (i.e., 0, 1, 2, 3, 4, 5, 6) amino acid substitutions. In a specific instance, the stitched GLP-1 peptide is based on the amino acid sequence of SEQ ID NO:59 with 0 to 3 (i.e., 0, 1, 2, 3) amino acid deletions from the N-terminus. In a specific instance, the stitched GLP-1 peptide is based on the amino acid sequence of SEQ ID NO:59 with 0 to 3 (i.e., 0, 1, 2, 3) amino acid deletions from the C-terminus. In a particular instance, the stitched peptide further comprises one or more of the modifications described in the sections “GLP-1 Peptides” and “Stitched Peptides” above. In certain instances, the 1 to 6 (i.e., 0, 1, 2, 3, 4, 5, 6) amino acids of the amino acid sequence set forth in SEQ ID NO:59 that are substituted by another amino acid comprise a substitution at position 2 of the amino acid sequence set forth in SEQ ID NO:59. In certain instances, the 1 to 6 (i.e., 0, 1, 2, 3, 4, 5, 6) amino acids of the amino acid sequence set forth in SEQ ID NO:59 that are substituted by another amino acid are on the GLP-1R-non-interacting face of the C-terminal portion of the GLP-1 helix of the amino acid sequence set forth in SEQ ID NO:59. Examples of amino acids in the C-terminal portion of GLP-1 that directly interact with GLP-1R are Ser18, Glu21, Ala24, Ala25, Lys26, Phe28, Ile29, Leu32, and Val33 (numbered with respect to the positions in the amino acid sequence set forth in SEQ ID NO:2). In some instances, the 1 to 3 amino acids in the amino acid sequence set forth in SEQ ID NO:59 that are removed from the N-terminus or are removed and replaced with 1 to 6 amino acids from the group consisting of alanine, D-alanine, α-aminoisobutyric acid, N-methyl glycine, serine, a substituted alanine, and a glycine derivative. In some instances, the 1 to 3 amino acids in the amino acid sequence set forth in SEQ ID NO:59 that are removed from the C-terminus or are removed and replaced with 1 to 6 amino acids from the group consisting of alanine, D-alanine, α-aminoisobutyric acid, N-methyl glycine, serine, a substituted alanine, and a glycine derivative. In some instances, 0 to 6 amino acids on the GLP-1R-non-interacting face of the C-terminal portion of the amino acid sequence set forth in SEQ ID NO:59 are substituted with an amino acid selected from the group consisting of alanine, D-alanine, α-aminoisobutyric acid, N-methyl glycine, serine, a substituted alanine, and a glycine derivative. In certain instances, the 1 to 6 amino acids of the amino acid sequence set forth in SEQ ID NO:59 that are substituted by another amino acid are on the GLP-1R-interacting face of the C-terminal portion of the GLP-1 helix of the amino acid sequence set forth in SEQ ID NO:59. In other instances, the 1 to 6 amino acids of the amino acid sequence set forth in SEQ ID NO:59 that are substituted by another amino acid are on the GLP-1R-non-interacting face of the C-terminal portion and the GLP-1R-interacting face of the C-terminal portion of the GLP-1 helix of the amino acid sequence set forth in SEQ ID NO:59. In certain instances, the 1 to 6 amino acids of the amino acid sequence set forth in SEQ ID NO:59 are substituted by an amino acid or amino acids selected from the group consisting of L-alanine, D-alanine, α-aminoisobutyric acid, N-methyl glycine, serine, a substituted alanine, and a glycine derivative.

In certain instances, the stitched GLP-1 peptide comprises a stitched GLP-1 peptide comprising or consisting of Compound (1), wherein [Xaa]_{w} is HGEGTFTSDV (SEQ ID NO:54), [Xaa]ₓ is SYLEGQ (SEQ ID NO:51), [Xaa]_{y} is AKEFIA (SEQ ID NO:52), and [Xaa]_{z} is LVKGRG (SEQ ID NO:56).

In another instance, the stitched GLP-1 peptide comprises a stitched (internally cross-linked) form of the amino acid sequence of SEQ ID NO:59 (e.g., the product of a ring-closing metathesis reaction on the amino acid sequence set forth in SEQ ID NO:59). In another instance, the stitched GLP-1 peptide consists of a stitched (internally cross-linked) form of the amino acid sequence of SEQ ID NO:59 (e.g., the product of a ring-closing metathesis reaction on the amino acid sequence set forth in SEQ ID NO:59).

### Methods of Treatment

The disclosure features methods of using any of the stitched peptides (or pharmaceutical compositions comprising said stitched peptides) described herein for the prophylaxis and/or treatment of diabetes, hyperglycemia, rapid gastric emptying, insulin resistance, or cardiovascular disease in a human subject in need thereof. The disclosure also features methods of using any of the stitched peptides (or pharmaceutical compositions comprising said stitched peptides) described herein for the prophylaxis and/or treatment of Alzheimer’s disease or Huntington’s disease in a human subject in need thereof. The disclosure also features methods of using any of the stitched peptides (or pharmaceutical compositions comprising said stitched peptides) described herein for increasing levels of cAMP in a human subject in need thereof (e.g., in GLP-1R-expressing cells in the human subject). The terms “treat” or “treating,” as used herein, refers to alleviating, inhibiting, or ameliorating the disease or condition from which the subject is suffering.

The stitched peptides (or compositions comprising the stitched peptides) described herein can be useful for treating a human subject with diabetes. In some instances, the diabetes is type 1 diabetes. In some instances, the diabetes is type 2 diabetes. The stitched peptides (or compositions comprising the stitched peptides) described herein can also be useful for treating a human subject with hyperglycemia.

In some instances, the stitched peptides (or compositions comprising the stitched peptides) described herein have the effect of improving blood glucose control, preserving beta-cell function, delaying gastric emptying, enabling weight loss, increasing insulin sensitivity, and/or mitigating cardiovascular disease. Thus, in some instances, the stitched peptides (or compositions comprising the stitched peptides) described herein can also be useful for treating a human subject with rapid gastric emptying, insulin resistance, or cardiovascular disease.

The stitched peptides (or compositions comprising the stitched peptides) described herein can be useful for treating a human subject with Alzheimer’s disease. The stitched peptides (or compositions comprising the stitched peptides) described herein can also be useful for treating a human subject with Huntington’s disease.

In some instances, the stitched peptides (or compositions comprising the stitched peptides) described herein can be useful in increasing cAMP levels in a human subject. In some instances, administration of the stitched peptides (or compositions comprising the stitched peptides) may result in an increase in cAMP levels in the human subject may increase by at least 5%, at least 10%, at least 20%, at least 30%, at least 40%, at least 50%, at least 60%, at least 70%, at least 80%, at least 90%, at least 100%, at least 150%, at least 200%, at least 300%, at least 400%, at least 500% as compared to cAMP levels in the human subject prior to (e.g., at least 1 hour, at least 1 day, at least 1 week before) administration to the human subject. In some instances, administration of the stitched peptides (or compositions comprising the stitched peptides) may result in an increase in cAMP levels in the human subject may increase by at most 5%, at most 10%, at most 20%, at most 30%, at most 40%, at most 50%, at most 60%, at most 70%, at most 80%, at most 90%, at most 100%, at most 150%, at most 200%, at most 300%, at most 400%, at most 500% as compared to cAMP levels in the human subject prior to (e.g., at least 1 hour, at least 1 day, at least 1 week before) administration to the human subject. In some instances the cAMP levels are evaluated within 1 week, within 2 weeks, within 3 weeks, within a month, within 2 months, or within 3 months of administration of the stitched peptide (or composition comprising the stitched peptide) to the human subject. In some instances, the increase in cAMP levels in the human subject is in GLP-1R-expressing cells in the human subject. Methods of assaying cAMP levels are known in the art (see, e.g., the working examples below).

In certain instances, the human subject in need thereof is administered a stitched peptide of Table 2. In certain instances, the human subject in need thereof is administered a stitched GLP-1 peptide comprising or consisting of a stitched peptide of the amino acid sequence set forth in SEQ ID NO:40 or a modified version thereof. In certain instances, the human subject in need thereof is administered a stitched GLP-1 peptide comprising or consisting of Compound (1), wherein [Xaa]_{w} is HJEGTFTSD (SEQ ID NO:45), [Xaa]ₓ is SSYLEG (SEQ ID NO:46), [Xaa]_{y} is AAKEFI (SEQ ID NO:47), and [Xaa]_{z} is WLVKGR (SEQ ID NO:48), wherein J is 2-aminoisobutyric acid. In certain instances, the human subject in need thereof is administered a stitched GLP-1 peptide comprising or consisting of a stitched peptide of the amino acid sequence set forth in SEQ ID NO:58 or a modified version thereof. In certain instances, the human subject in need thereof is administered a stitched GLP-1 peptide comprising or consisting of Compound (1), wherein [Xaa]_{w} is HJEGTFTSD (SEQ ID NO:45), [Xaa]ₓ is SSYLEG (SEQ ID NO:46), [Xaa]_{y} is AAKEFI (SEQ ID NO:47), and [Xaa]_{z} is WLVKGRG (SEQ ID NO:55), wherein J is 2-aminoisobutyric acid. In certain instances, the human subject in need thereof is administered a stitched GLP-1 peptide comprising or consisting of a stitched peptide of the amino acid sequence set forth in SEQ ID NO:33 or a modified version thereof. In certain instances, the human subject in need thereof is administered a stitched GLP-1 peptide comprising or consisting of Compound (1), wherein [Xaa]_{w} is HGEGTFTSD (SEQ ID NO:49), [Xaa]ₓ is SSYLEG (SEQ ID NO:46), [Xaa]_{y} is AAKEFI (SEQ ID NO:47), and [Xaa]_{z} is WLVKGR (SEQ ID NO:48). In certain instances, the human subject in need thereof is administered a stitched GLP-1 peptide comprising or consisting of a stitched peptide of the amino acid sequence set forth in SEQ ID NO:57 or a modified version thereof. In certain instances, the human subject in need thereof is administered a stitched GLP-1 peptide comprising or consisting of Compound (1), wherein [Xaa]_{w} is HGEGTFTSD (SEQ ID NO:49), [Xaa]ₓ is SSYLEG (SEQ ID NO:46), [Xaa]_{y} is AAKEFI (SEQ ID NO:47), and [Xaa]_{z} is WLVKGRG (SEQ ID NO:55). In certain instances, the human subject in need thereof is administered a stitched GLP-1 peptide comprising or consisting of a stitched peptide of the amino acid sequence set forth in SEQ ID NO:41 or a modified version thereof. In certain instances, the human subject in need thereof is administered a stitched GLP-1 peptide comprising or consisting of Compound (1), wherein [Xaa]_{w} is HJEGTFTSDV (SEQ ID NO:50), [Xaa]ₓ is SYLEGQ (SEQ ID NO:51), [Xaa]_{y} is AKEFIA (SEQ ID NO:52), and [Xaa]_{z} is LVKGR (SEQ ID NO:53), wherein J is 2-aminoisobutyric acid. In certain instances, the human subject in need thereof is administered a stitched GLP-1 peptide comprising or consisting of a stitched peptide of the amino acid sequence set forth in SEQ ID NO:60 or a modified version thereof. In certain instances, the human subject in need thereof is administered a stitched GLP-1 peptide comprising or consisting of Compound (1), wherein [Xaa]_{w} is HJEGTFTSDV (SEQ ID NO:50), [Xaa]ₓ is SYLEGQ (SEQ ID NO:51), [Xaa]_{y} is AKEFIA (SEQ ID NO:52), and [Xaa]_{z} is LVKGRG (SEQ ID NO:56), wherein J is 2-aminoisobutyric acid. In certain instances, the human subject in need thereof is administered a stitched GLP-1 peptide comprising or consisting of a stitched peptide of the amino acid sequence set forth in SEQ ID NO:34 or a modified version thereof. In certain instances, the human subject in need thereof is administered a stitched GLP-1 peptide comprising or consisting of Compound (1), wherein [Xaa]_{w} is HGEGTFTSDV (SEQ ID NO:54), [Xaa]ₓ is SYLEGQ (SEQ ID NO:51), [Xaa]_{y} is AKEFIA (SEQ ID NO:52), and [Xaa]_{z} is LVKGR (SEQ ID NO:53). In certain instances, the human subject in need thereof is administered a stitched GLP-1 peptide comprising or consisting of a stitched peptide of the amino acid sequence set forth in SEQ ID NO:59 or a modified version thereof. In certain instances, the human subject in need thereof is administered a stitched GLP-1 peptide comprising or consisting of Compound (1), wherein [Xaa]_{w} is HGEGTFTSDV (SEQ ID NO:54), [Xaa]ₓ is SYLEGQ (SEQ ID NO:51), [Xaa]_{y} is AKEFIA (SEQ ID NO:52), and [Xaa]_{z} is LVKGRG (SEQ ID NO:56).

In some instances, the human subject has type 1 diabetes. In some instances, the human subject has type 2 diabetes. In some instances, the human subject has hyperglycemia.

In some instances, the human subject has gastric emptying. In some instances, the human subject has insulin resistance. In some instances, the human subject has cardiovascular disease.

In some instances, the human subject has Alzheimer’s disease.

In some instances, the human subject has Huntington’s disease.

In general, methods include selecting a subject and administering to the subject an effective amount of one or more of the stitched peptides herein, e.g., in or as a pharmaceutical composition, and optionally repeating administration as required for the prophylaxis or treatment of diabetes, hyperglycemia, gastric emptying, insulin resistance, cardiovascular disease, Alzheimer’s disease, or Huntington’s disease and can be administered orally or intravenously. A subject can be selected for treatment based on, e.g., determining that the subject has diabetes, hyperglycemia, gastric emptying, insulin resistance, cardiovascular disease, Alzheimer’s disease, or Huntington’s disease.

Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject’s disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. For example, effective amounts can be administered at least once.

### Pharmaceutical Compositions

One or more of any of the stitched peptides described herein (or pharmaceutically acceptable salts thereof) can be formulated for use as or in pharmaceutical compositions. The pharmaceutical compositions may be used in the methods of treatment described herein (see above). In certain instances, the pharmaceutical composition comprises a peptide comprising or consisting of an amino acid sequence that is identical to an amino acid sequence set forth in Table 2, except for 1 to 10, 1 to 9, 1 to 8, 1 to 7, 1 to 6, 1 to 5, 1 to 4, 1 to 3, 1 to 2, or 1 amino acid substitution, insertion, or deletion. These changes to the amino acid sequences can be made on the GLP-1R-non-interacting alpha-helical face of the GLP-1 C-terminal portion of these peptides (i.e., to the amino acids that do not engage with GLP-1R) and/or on the GLP-1R-interacting alpha-helical face of the GLP-1 C-terminal portion of these peptides (i.e., to the amino acids that directly interact with GLP-1R) and/or in the GLP-1 N-terminal portion of these peptides. Examples of amino acids in the C-terminal portion of GLP-1 that directly interact with GLP-1R are Ser18, Glu21, Ala24, Ala25, Lys26, Phe28, Ile29, Leu32, and Val33 (numbered with respect to the positions in the amino acid sequence set forth in SEQ ID NO:2). Such compositions can be formulated or adapted for administration to a subject via any route, e.g., any route approved by the Food and Drug Administration (FDA). Exemplary methods are described in the FDA’s CDER Data Standards Manual, version number 004 (which is available at fda.give/cder/dsm/DRG/drg00301.htm). For example, compositions can be formulated or adapted for administration by inhalation (e.g., oral and/or nasal inhalation (e.g., via nebulizer or spray)), injection (e.g., intravenously, intra-arterial, subdermally, intraperitoneally, intramuscularly, and/or subcutaneously); and/or for oral administration, transmucosal administration, and/or topical administration (including topical (e.g., nasal) sprays and/or solutions).

In some instances, pharmaceutical compositions can include an effective amount (e.g., a therapeutically effective amount) of one or more stitched peptides. The terms “effective amount” and “effective to treat,” as used herein, refer to an amount or a concentration of one or more compounds (e.g., stitched peptide) or a pharmaceutical composition described herein utilized for a period of time (including acute or chronic administration and periodic or continuous administration) that is effective within the context of its administration for causing an intended effect or physiological outcome (e.g., treatment of diabetes, hyperglycemia, rapid gastric emptying, insulin resistance, cardiovascular disease, Alzheimer’s disease, or Huntington’s disease).

Pharmaceutical compositions of this invention can include one or more peptides and any pharmaceutically acceptable carrier and/or vehicle. In some instances, pharmaceuticals can further include one or more additional therapeutic agents in amounts effective for achieving a modulation of disease or disease symptoms.

The term “pharmaceutically acceptable carrier or adjuvant” refers to a carrier or adjuvant that may be administered to a patient, together with a compound (e.g., stitched peptide) of this disclosure, and which does not destroy the pharmacological activity thereof and is nontoxic when administered in doses sufficient to deliver a therapeutic amount of the compound (e.g., stitched peptide).

Pharmaceutically acceptable carriers, adjuvants and vehicles that may be used in the pharmaceutical compositions of this disclosure include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of stitched peptides described herein.

The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intra-cutaneous, intra-venous, intra-muscular, intra-articular, intra-arterial, intra-synovial, intra-sternal, intra-thecal, intra-lesional and intra-cranial injection or infusion techniques.

Pharmaceutical compositions can be in the form of a solution or powder for inhalation and/or nasal administration. Such compositions may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer’s solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

Pharmaceutical compositions can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

Alternatively or in addition, pharmaceutical compositions can be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

In some instances, one or more stitched peptides disclosed herein can be conjugated, for example, to a carrier protein. Such conjugated compositions can be monovalent or multivalent. For example, conjugated compositions can include one stitched peptide disclosed herein conjugated to a carrier protein. Alternatively, conjugated compositions can include two or more stitched peptides disclosed herein conjugated to a carrier.

As used herein, when two entities are “conjugated” to one another they are linked by a direct or indirect covalent or non-covalent interaction. In certain instances, the association is covalent. In other instances, the association is non-covalent. Non-covalent interactions include hydrogen bonding, van der Waals interactions, hydrophobic interactions, magnetic interactions, electrostatic interactions, etc. An indirect covalent interaction is when two entities are covalently connected, optionally through a linker group.

Carrier proteins can include any protein that increases or enhances immunogenicity in a subject. Exemplary carrier proteins are described in the art (see, e.g., Fattom et al., Infect. Immun., 58:2309-2312, 1990; Devi et al., Proc. Natl. Acad. Sci. USA 88:7175-7179, 1991; Li et al., Infect. Immun. 57:3823-3827, 1989; Szu et al., Infect. Immun. 59:4555-4561,1991; Szu et al., J. Exp. Med. 166:1510-1524, 1987; and Szu et al., Infect. Immun. 62:4440-4444, 1994). Polymeric carriers can be a natural or a synthetic material containing one or more primary and/or secondary amino groups, azido groups, or carboxyl groups. Carriers can be water soluble.

### Methods of Making Structurally-Stabilized Peptides

Also provided herein are methods of making structurally-stabilized peptides (e.g., a structurally-stabilized peptide described herein such as a stitched peptide). In some instances, the method comprises (a) providing a peptide described herein, wherein the peptide comprises at least three stitching amino acids, and (b) performing a ring-closing metathesis reaction. In some instances, the method further comprises formulating the stitched peptide as a pharmaceutical composition.

### Methods of Screening Structurally-Stabilized Peptides

This disclosure features a method of screening for a stabilized peptide. The method involves providing a cell expressing a detectably labeled-GLP-1R. The method further involves contacting the cell with one or more stabilized (e.g., stitched) peptides and selecting the stabilized peptide that internalizes the detectably labeled-GLP-1R. In some cases, the cell is a U2OS cell, a COS cell, a Hela cell, a 293 cell, a 293T cell, or a NIH3T3 cell. In certain cases, the GLP-1R is detectably labeled with a fluorescent polypeptide. In some instances internalization is assessed by imaging. In certain cases, the imaging is epifluorescence or confocal imaging (e.g., by quantifying flurescent cytosolic punctae). In some instances the level of internalization of the stabilized peptide is compared with an unstapleds/unstitched GLP-1 peptide (e.g., SEQ ID NO: 3, 4, 31, 38, 69, or 70) and the stabilized peptide that has increased internalization relative to the unstapleds/unstitched GLP-1 peptide is selected. In some instances, the selected stabilized peptide is further tested for proteolytic resistance and/or functional benefit (e.g., glucose tolerance test).

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Preparation of GLP-1 Stapled Peptides

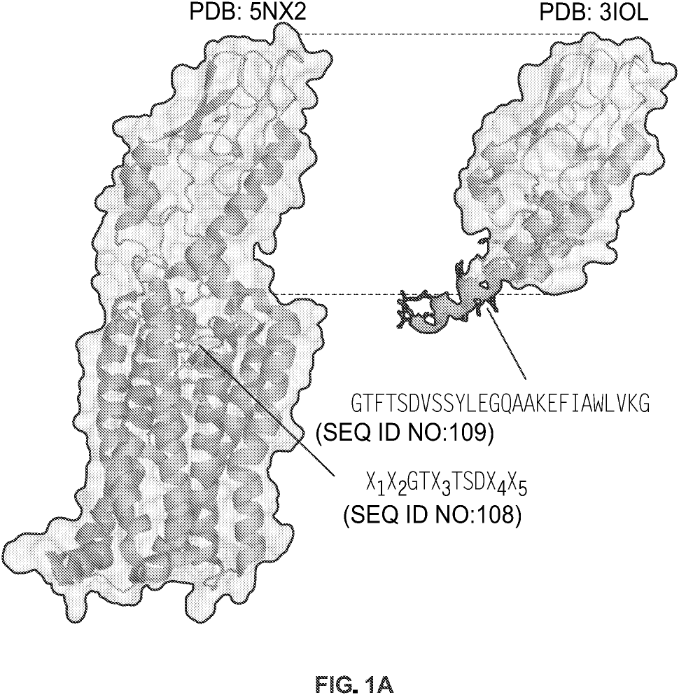

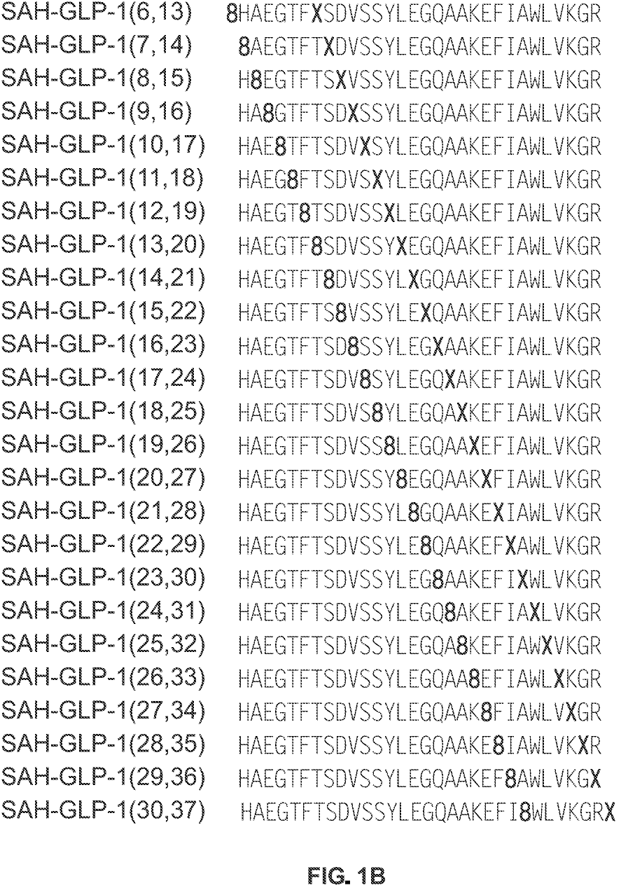

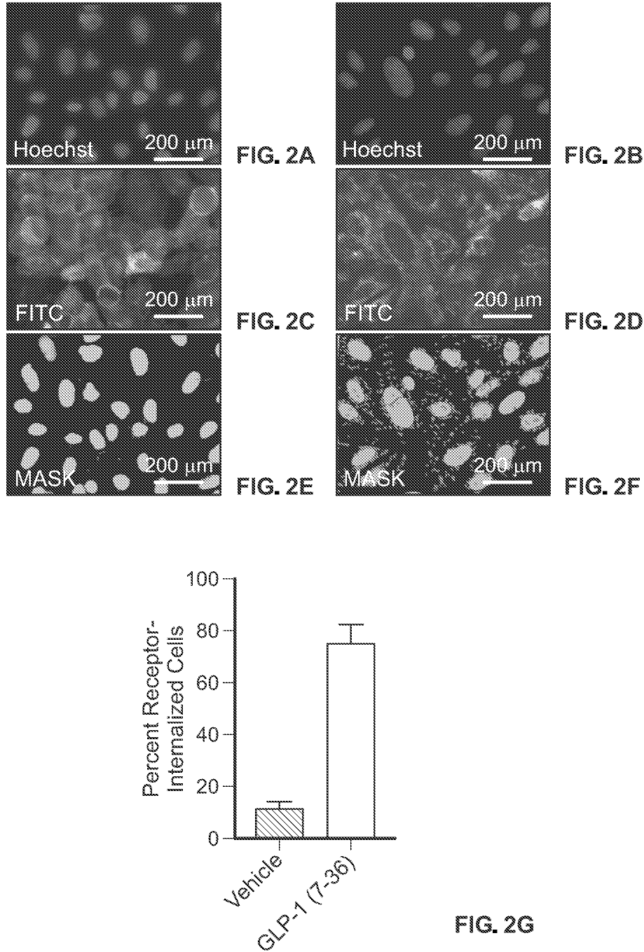

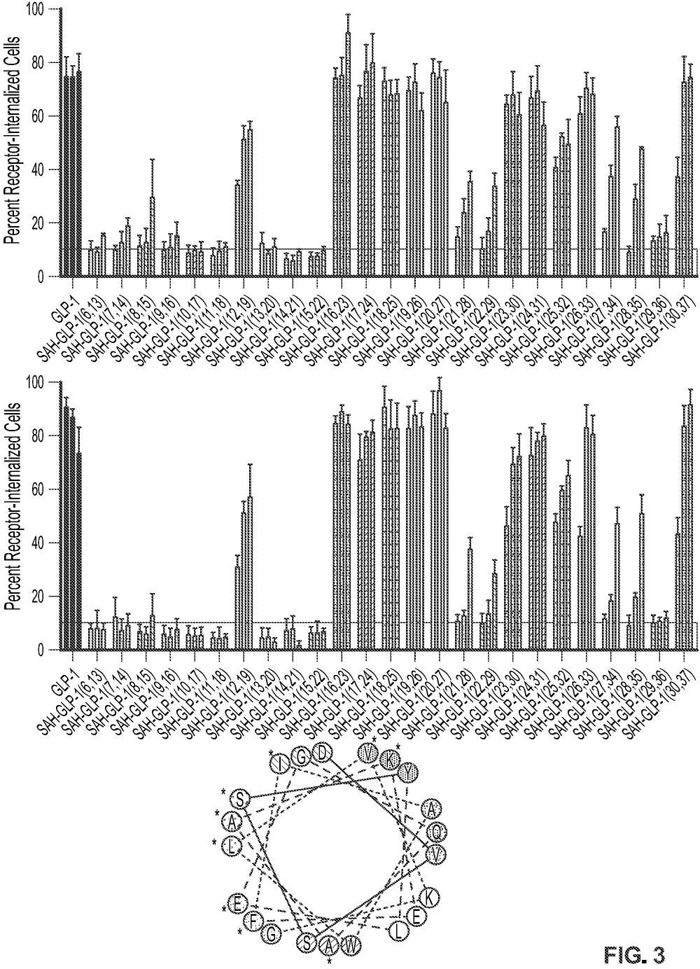

The helical structure of the biologically-active GLP-1 peptide is comprised of an N-terminal segment that sits deep within the GLP-1R binding pocket and a C-terminal portion that participates in a more traditional helix-in-groove interaction with the extracellular domain of GLP-1R (FIG. 1A) (Underwood et al., Journal of Biological Chemistry. 2010;285(1):723-30; Jazayeri et al., Nature. 2017;546(7657)254-258; Song et al., Nature. 2017;546(7657):312-315). An i, i+7 staple scan of GLP-1-(7-37) (SEQ ID NO:3) was performed (FIG. 1B). The structurally-stabilized alpha-helices of GLP-1 (SAH-GLP-1) were screened for functional GLP-1R binding activity using U2OS cells stably expressing GFP-GLP-1R. Upon receptor engagement by GLP-1, the GFP-GLP-1R:GLP-1 complex is internalized and can be detected and quantified by high-content epifluorescence microscopy. Using a single dose screening approach, each i, i+7-stapled GLP-1 peptide (FIG. 1B) was evaluated in a GLP-1R internalization assay. FIG. 2A-FIG. 2G show representative data for screening with GLP-1(7-36) peptide (SEQ ID NO:4). GFP-GLP-1R-expressing U2OS cells demonstrated fluorescence of the plasma membrane at baseline (FIG. 2A, FIG. 2C, and FIG. 2E), but upon GLP-1 peptide exposure (SEQ ID NO:4) (FIG. 2B, FIG. 2D, and FIG. 2F), the receptor was internalized, as reflected by fluorescent cytosolic punctae that can be quantified by high-content epifluorescence imaging. FIG. 2G shows the percentage of cells containing internalized GFP-GLP-1R under each condition (vehicle vs. 0.5 µM GLP-1). The screen uncovered three striking structure-function principles. First, 9 of the 10 N-terminal staple positions were incompatible with GLP-1R binding and internalization (FIG. 3; see SAH-GLP-1(6,13) through SAH-GLP-1(11,18) and SAH-GLP-1(13,20) through SAH-GLP-1(15,22), i.e., SEQ ID NOs: 6-11 and 13-14, respectively). These data are consistent with the requisite burial of the peptide N-terminus in the receptor and the staple producing steric hindrance or replacing residues critical to agonistic function. Second, tolerance of staples at up to five consecutive positions within the extracellular domain-binding portion of GLP-1, namely SAH-GLP-1(16,23) through SAH-GLP-1(20,27) (i.e., SEQ ID NOs: 16-20, respecitvely), indicated that no discrete surface in this region is essential to GLP-1R activation. Third, discrete pairs of heptads situated along the length of the GLP-1 helix (e.g., SAH-GLP-1(16,23)/SAH-GLP-1(23,30); SAH-GLP-1(17,24)/SAH-GLP-1(24,31); SAH-GLP-1(18,25)/SAH-GLP-1(25,32); SAH-GLP-1(19,26)/SAH-GLP-1(26,33)) showed patterns of tolerability (FIG. 3), suggesting that long-range α-helical reinforcement might be accomplished by double i, i+7 stitching. Moreover, in the C-terminal portion of the GLP-1 peptide there were several examples of staples that did not work as well regardless of whether the staple was on the GLP-1R-interacting face or on the GLP-1R-non-interacting face. For example, each of SAH-GLP-1(21,28), SAH-GLP-1(22,29), and SAH-GLP-1(28,34) contain a staple on the GLP-1R-interacting face and the resulting stapled peptide was not as active. However, SAH-GLP-1(27,34) contains a staple on the GLP-1R-non-interacting face and likewise was not as active.

Interestingly, to Applicant’s knowledge, this type of all-hydrocarbon crosslink spanning four helical turns has not been biologically tested to date in the context of a bioactive peptide and appeared especially well-suited to GLP-1 peptide design based upon the single i, i+7 staple scanning structure-activity relationship (SAR) results.

### Example 2: Preparation of I, I+7 Stitched GLP-1 Peptides

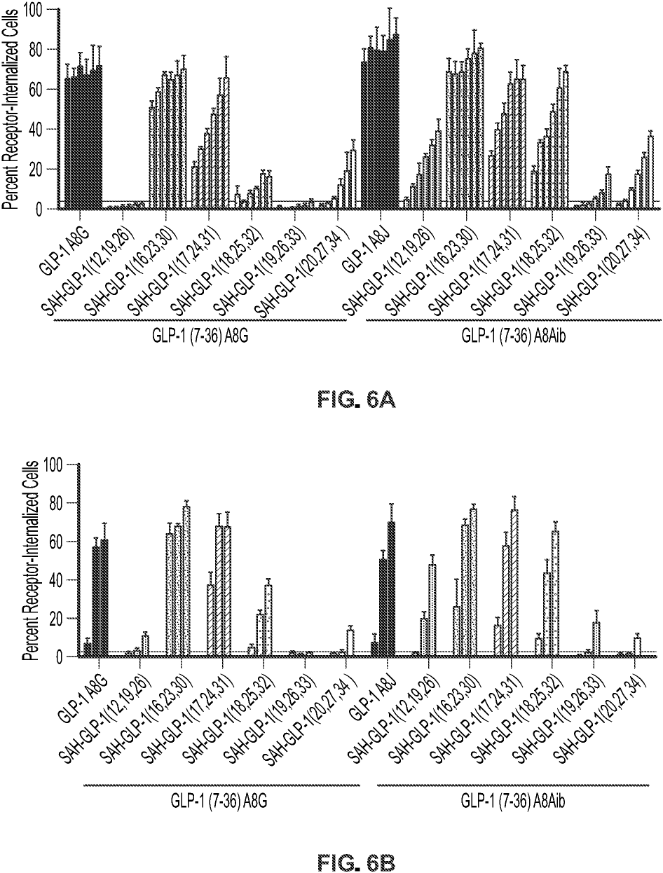

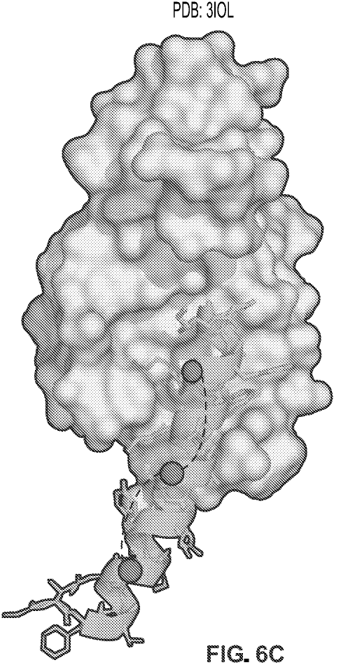

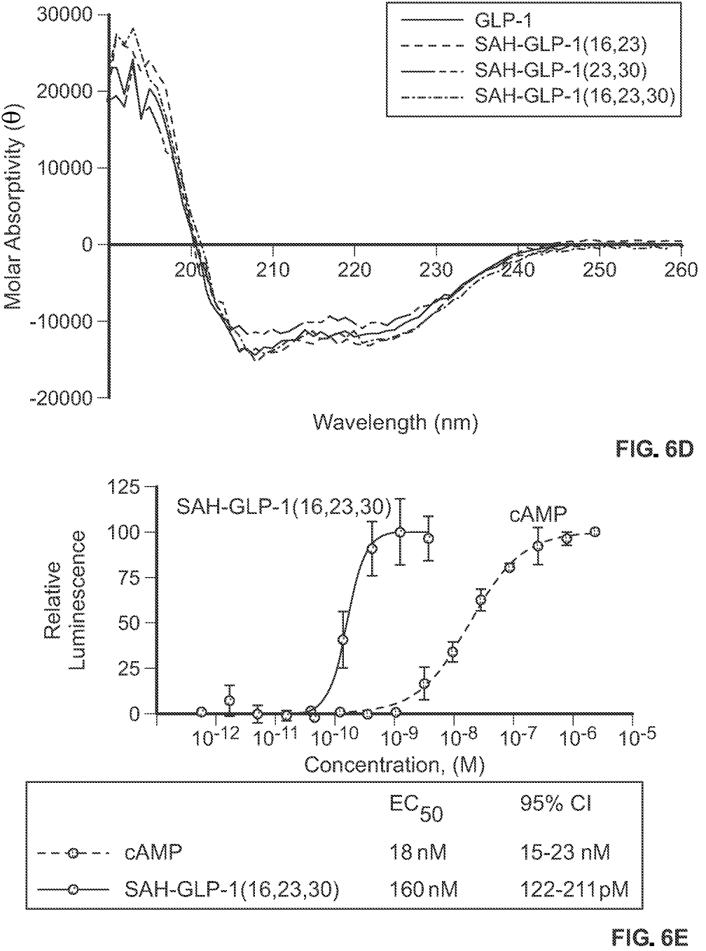

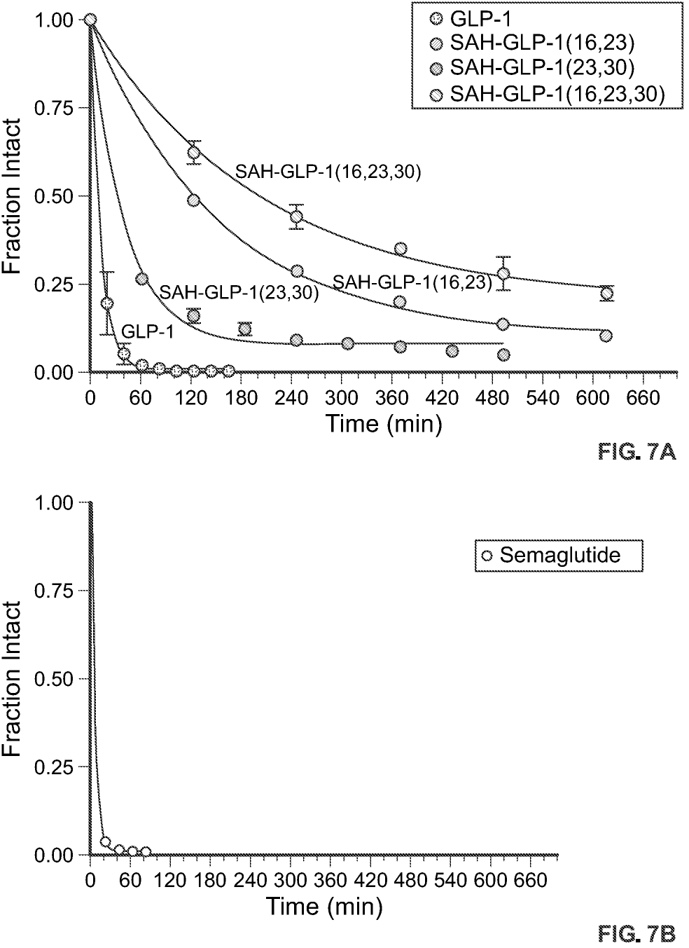

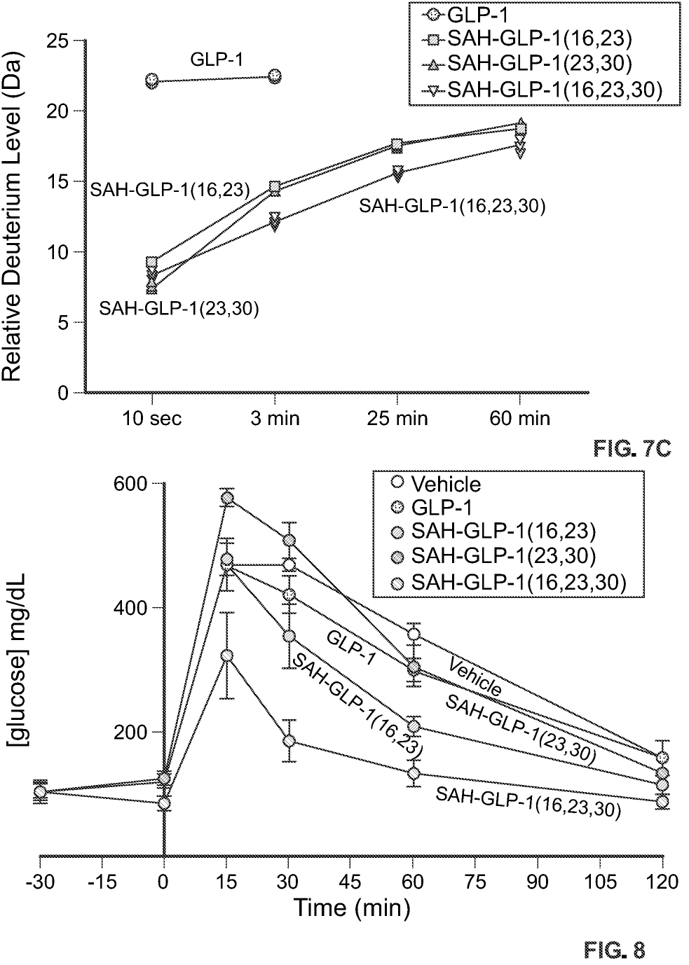

To generate i, i+ 7-stitched constructs corresponding to the pairs of singly stapled residues (FIG. 1B) located along a contiguous face of the GLP-1 helix, the following were installed from C-terminal to N-terminal of GLP-1 (7-37): an S-octenyl alanine (S8), a bis-pentenyl glycine (Bis-5), and an R-octenyl alanine (R8). These non-natural amino acids were installed to afford compatible i, i+7 stapling between the olefins of S8-R5 and S5-R8 non-natural amino acid pairs within the peptide sequence (FIG. 4). Ala8 (numbered according to the GLP-1 peptide of SEQ ID NO:2) was replaced with either Glycine (G) or aminoisobutyric acid (Aib/J) in the stitched library. FIG. 5 depicts the sequences of the stitched peptides (prior to the cross-linking reaction) along with their non-stitched parental peptides. Comparing the activity of single and stitched compounds in the GLP-1R internalization assay, single staples were generally less disruptive to internalization (comparing FIG. 3 and FIG. 6A), whereas the structure-activity relationship was more restrictive for the stitched analogs (FIG. 6A). Strikingly, 9 of 10 i, i+7 singly stapled peptides that localized the staple within the N-terminal portion of the peptide sequence (amino acids 6-15 of SEQ ID NO:2) were inactive, regardless of what face of the helix the staple was located to, and consistent with the requisite burial of the native sequence within the receptor binding site (FIG. 1A, left). Surprisingly, the structure-activity relationship for the i, i+7 singly stapled peptides that localized the staple within the C-terminal portion of the peptide sequence (starting with amino acid 16 of SEQ ID NO:2 and beyond), showed 10 of 15 constructs with potent activity and a pattern that was favorable for select staples that were either on the interacting or non-interacting face (cf. constructs with staple positions 16,23; 17,24; 18,25; and 19;26; FIG. 3). Notably, however, one of the stitches that best recapitulated the internalization activity of the unstapled control peptide (FIG. 6A) and even improved upon the latter (FIG. 6B), as found in the SAH-GLP-1(16,23,30) constructs, was located on the non-interacting face of the GLP-1 helix (FIG. 6C). The second most active constructs contained the 17,24,31 stitch, which is likewise located on the non-interacting face, yet the third most active constructs contain the 18,25,32 stitch, which faces the binding site. To summarize: (i) some peptides having the stitch located on the non-interacting face were shown to be active (see SAH-GLP-1(16,23,30) and SAH-GLP-1(17,24,31)), while another peptide having the stitch on the non-interacting face was not as active (see SAH-GLP-1(20,27,34)); (ii) one peptide having the stitch located on the interacting face was shown to be active (see SAH-GLP-1(18,25,32)), while another peptide having the stitch on the interacting face was not as active (see SAH-GLP-1(19,23,33)); and (iii) one peptide having the stitch partially on the interacting face did not work as well (see SAH-GLP-1(12,19,26)). Taken together, these data highlight the unpredictability of the structure-activity relationships for stapled and stitched constructs based on the GLP-1 template peptide. If one of the two i, i+7 staples was disfavored as a single-stapled construct (see FIG. 3), stitching did not salvage the activity and the influence of the disruptive stapled prevailed. In certain cases where the single i, i+7 staples were tolerated on the interacting face (e.g., SAH-GLP-1(19,26) and SAH-GLP-1(26,33)), stitching was not favorable (FIG. 6A, FIG. 6B). In another example involving positions 18,25 and 25,32, both of which were favorable as single i, i+7 staples and localized to the interacting face, stitching preserved activity (FIG. 6A, FIG. 6B). However, if individual staples were tolerated and localized to the non-interacting surface (e.g., SAH-GLP-1(16,23) and SAH-GLP-1(23,30)) or SAH-GLP-1(17,24) and SAH-GLP-1(24,31), the stitched peptides showed the most favorable activity, such as for the constructs bearing 16,23,30 and 17,24,31 stitches (FIG. 6A, FIG. 6B, FIG. 6C). Overall, the stitched construct that best preserved and even improved upon the biological properties of wild-type GLP-1 in the receptor internalization assay was SAH-GLP-1(16,23,30) (SEQ ID NO:40) (FIGS. 6A, 6B, 6C). In addition, this construct, and the corresponding single-stapled i, i+7 peptides, were further shown to maintain the alpha-helical structure of the natural (unstapled) GLP-1 peptide in solution (FIG. 6D). In advance of subjecting SAH-GLP-(16,23,30) (SEQ ID NO:40) to a battery of proteolytic and in vivo assays, it was confirmed that, in addition to functioning as an agonist in the screening GLP-1R binding and internalization assay, SAH-GLP-1(16,23,30) A8J (SEQ ID NO:40) was capable of increasing cAMP levels in a gold standard GLP-1R signal transduction assay. GLP-1R-expressing CHO-K1 cells were treated with a serial dilution of SAH-GLP-1(16,23,30) A8J (SEQ ID NO:40) and a dose-responsive cAMP induction (EC₅₀, 160 pM) was observed, as detected by cAMP Hunter eXpress luminescence assay (FIG. 6D). Based on the collective favorable properties of SAH-GLP-1(16,23,30) (SEQ ID NO:40), this construct was advanced to proteolytic and in vivo testing.

### Example 3: Proteolytic Resistance of Stapled and Stitched GLP-1 Peptides

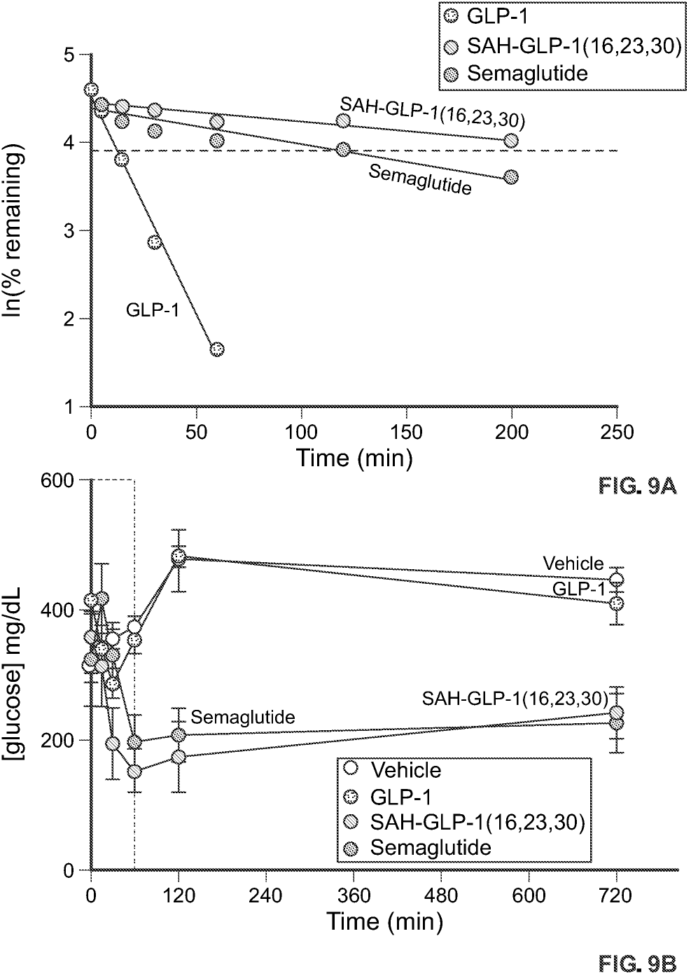

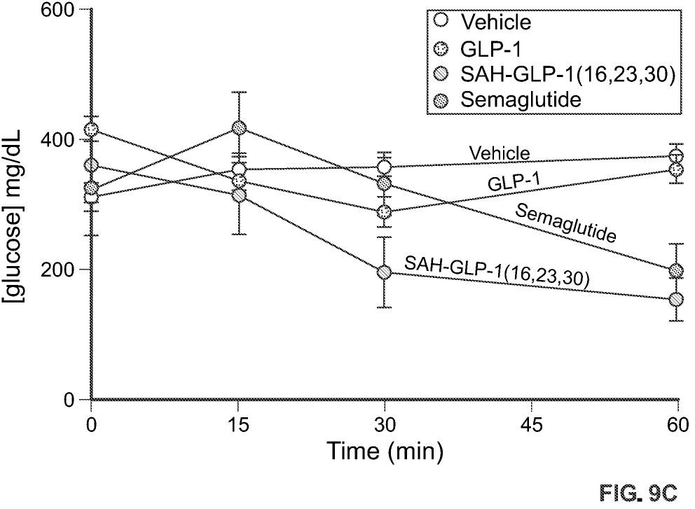

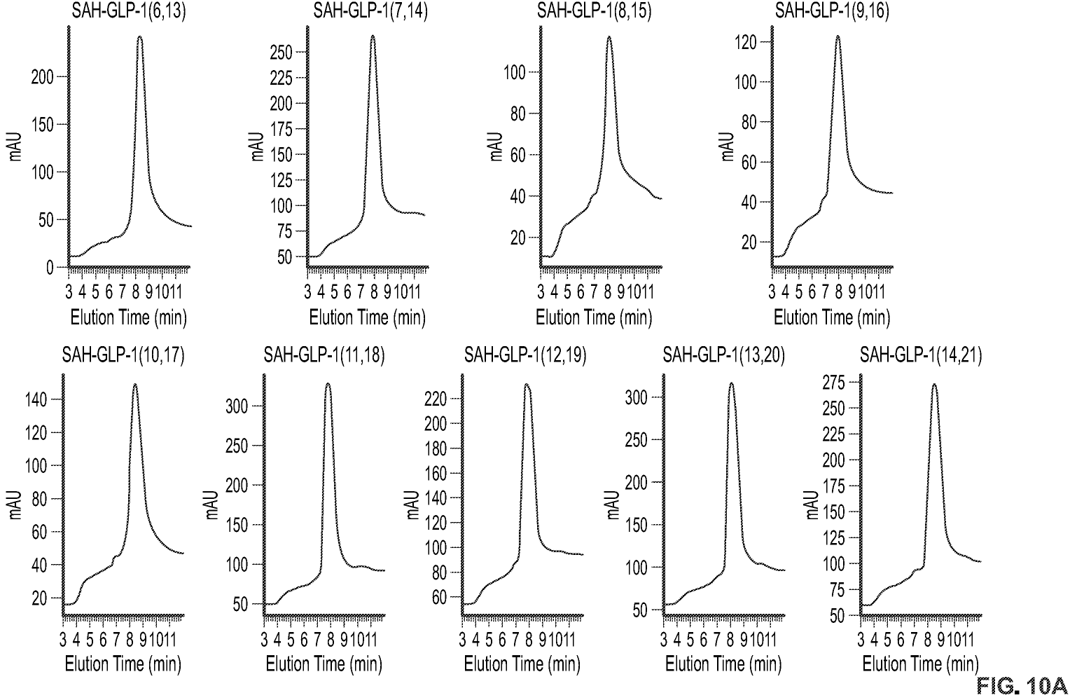

To evaluate the potential benefit of stitching in maximizing protease resistance, SAH-GLP-1(16,23,30) (SEQ ID NO:40), its single-stapled analogs, and wild-type GLP-1 were subjected to proteolytic digestion by proteinase K, a broad-spectrum protease that hydrolyzes the peptide bond adjacent to the carboxyl group of hydrophobic amino acids (FIG. 7A). Each of the peptides tested in FIG. 7A have the A8Aib substitution. As expected, the control GLP-1 peptide was rapidly digested in vitro with a half-life of less than 10 minutes. The single i, i+7 staples showed 3- to 12-fold improvement compared to wild-type, with respective half-lives of 120 min and 30 min for SAH-GLP-1(16,23) and SAH-GLP-1(23,30) (FIG. 7A). The stitched peptide (SAH-GLP-1(16,23,30) (SEQ ID NO:40) was the most proteolytically resistant construct, with a half-life of 220 min (FIG. 7A) that bested the control peptide by over 20-fold and the singly stapled peptides by 2-7 fold. Notably, the FDA-approved GLP-1 peptide drug semaglutide, was rapidly proteolyzed under the identical conditions, highlighting the capacity of stapling and stitching to vastly improve upon the proteolytic stability of natural and unnatural GLP-1 peptide sequences that lack a staple or stitch (FIG. 7B).

### Example 4: Comparative Structural Dynamics of Stapled and Stitched GLP-1 Peptides

To probe comparative structural dynamics of stitched GLP-1 peptides, the SAH-GLP-1(16,23), SAH-GLP-1(23,30), and SAH-GLP-1(16,23,30) A8J peptide panel was analyzed by hydrogen-deuterium exchange mass spectrometry (HXMS), which detects changes in peptide or protein conformation over time based on differences in hydrogen-bonding and solvent exposure (Engen, J. R. (2009) Analysis of protein conformation and dynamics by hydrogen/deuterium exchange MS, Anal Chem 81, 7870-7875, which is incorporated by reference herein in its entirety). After 10 seconds of D₂O exposure, GLP-1 peptides bearing single or stitched i, i+7 staples showed reduced deuterium exchange by 2.4-3 fold compared to the template peptide (FIG. 7C), highlighting the conformational rigidity conferred by the staples and a structure-dynamics relationship not detectable by CD averaging (FIG. 6D). What’s more, monitoring deuterium exchange over time revealed that i, i+7 stitching consistently conferred more protection at 3 minutes, 25 minutes, and 60 minutes when compared to single i, i+7 stapling alone (FIG. 7C).

### Example 5: Evaluation of in vivo Function of Stapled and Stitched GLP-1 Peptides

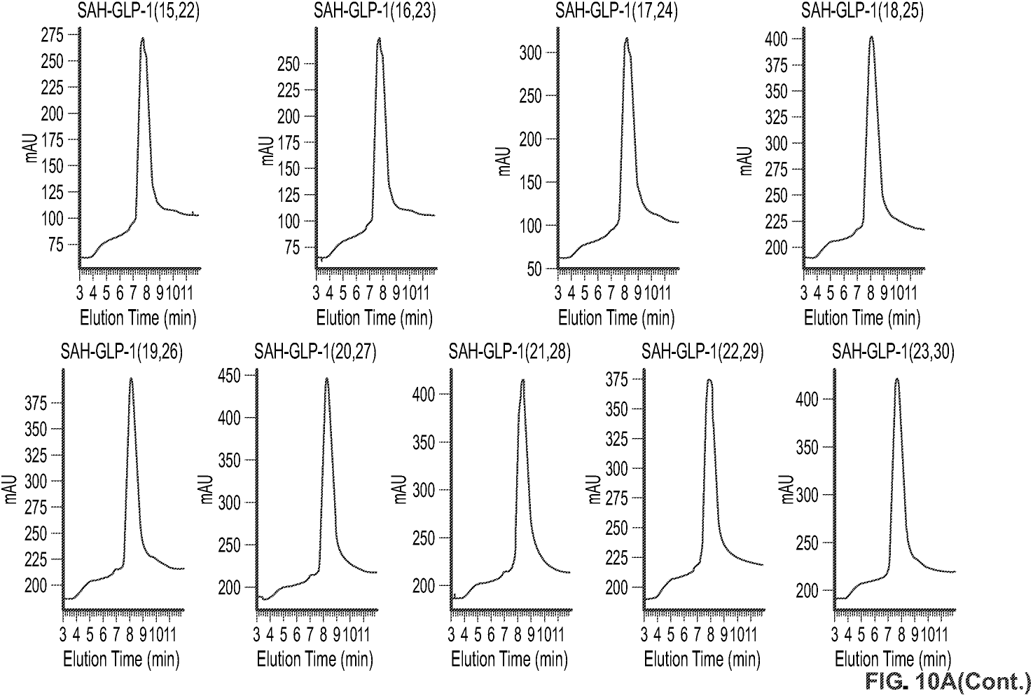

To evaluate whether the relative proteolytic stability of SAH-GLP-1(16,23,30) translated into a functional benefit in vivo, glucose tolerance tests in mice were performed. Mice (n=8 per arm) were fasted overnight and administered the corresponding panel of peptides at 10 nmol/kg intraperitoneally followed by a glucose bolus (2 g/kg) 30 minutes later, and then serial serum glucose monitoring was performed over time. In accordance with the protease resistance testing, wild-type GLP-1 and SAH-GLP-1(23,30) were the least effective at controlling the induced hyperglycemia, whereas SAH-GLP-1(16,23,30) followed by SAH-GLP-1(16,23) produced the greatest benefit (FIG. 8). Indeed, SAH-GLP-1(16,23,30) lowered the maximum serum glucose concentration by nearly half of the vehicle control, and achieved euglycemia within 60 minutes of the glucose bolus (FIG. 8).

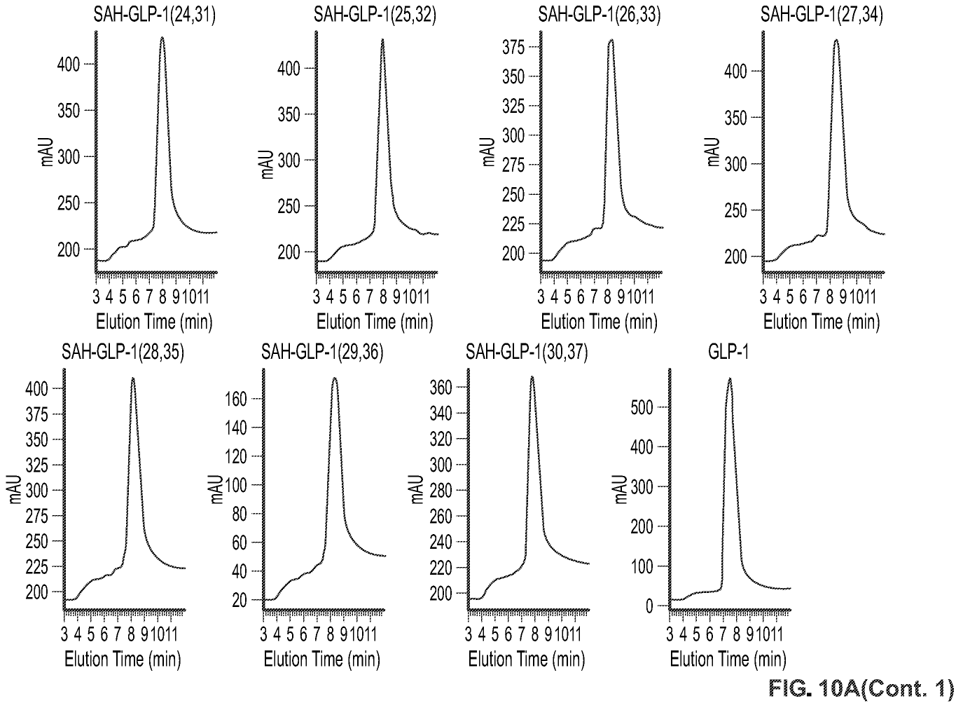

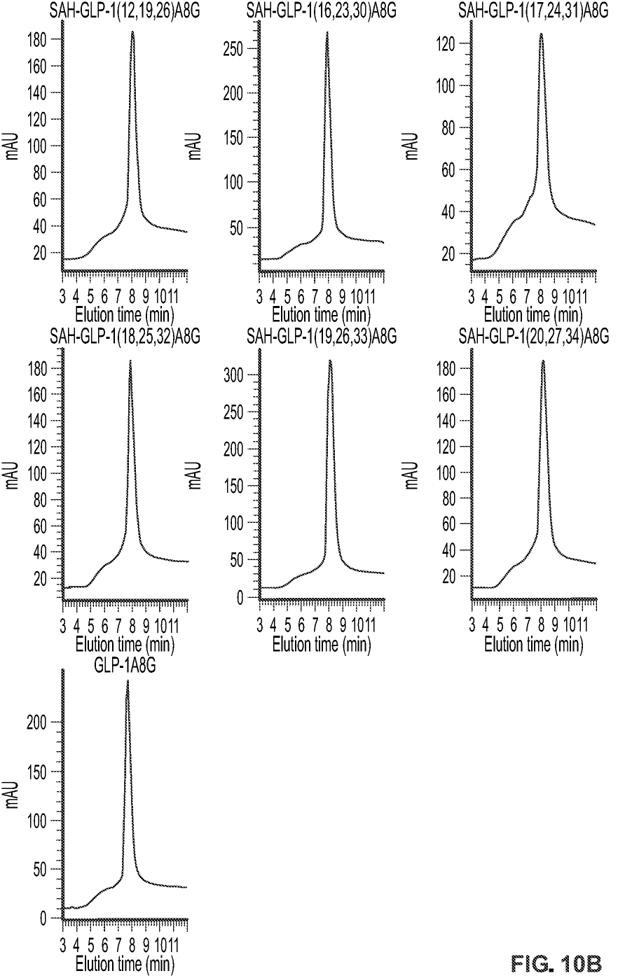

The activity of SAH-GLP-1(16,23,30) (SEQ ID NO:40) was compared to the FDA-approved GLP-1 analog semaglutide (Lau et al., Journal of Medicinal Chemistry. 2015;58(18):7370-80) in Leprdb mice, which manifest hyperglycemia at baseline. Whereas the in vivo stability of semaglutide was optimized based on mutating the DPP4 proteolytic site and appending a lipid moiety to maximize albumin binding (Lau et al., Journal of Medicinal Chemistry. 2015;58(18):7370-80), here we instead combined DPP4 site mutagenesis with structural reinforcement by i, i+7 stitching, which causes direct resistance to proteolysis (FIGS. 7A-B). Both approaches result in marked improvements in comparative ex vivo mouse plasma stability testing, yielding half-lives 12-fold (semaglutide) and 23-fold (SAH-GLP-1-A8J[16,23,30)]) greater than the natural GLP-1 peptide (FIG. 9A). Notably, the half-life of SAH-GLP-1-A8J(16,23,30) was nearly double that of semaglutide, highlighting the improvement in serum stability conferred by the stitching approach (FIG. 9A). Strikingly, single IP injection (10 nmol/kg) of SAH-GLP-1(16,23,30) (SEQ ID NO:40) produced a more rapid reduction in serum glucose compared to semaglutide, with both compounds sustaining a similar 2-2.5 fold reduction in glucose level compared to vehicle and wild-type GLP-1 for the 12-hour duration of the experiment (FIG. 9B and FIG. 9C).

### Materials and Methods Used in Examples 1-5

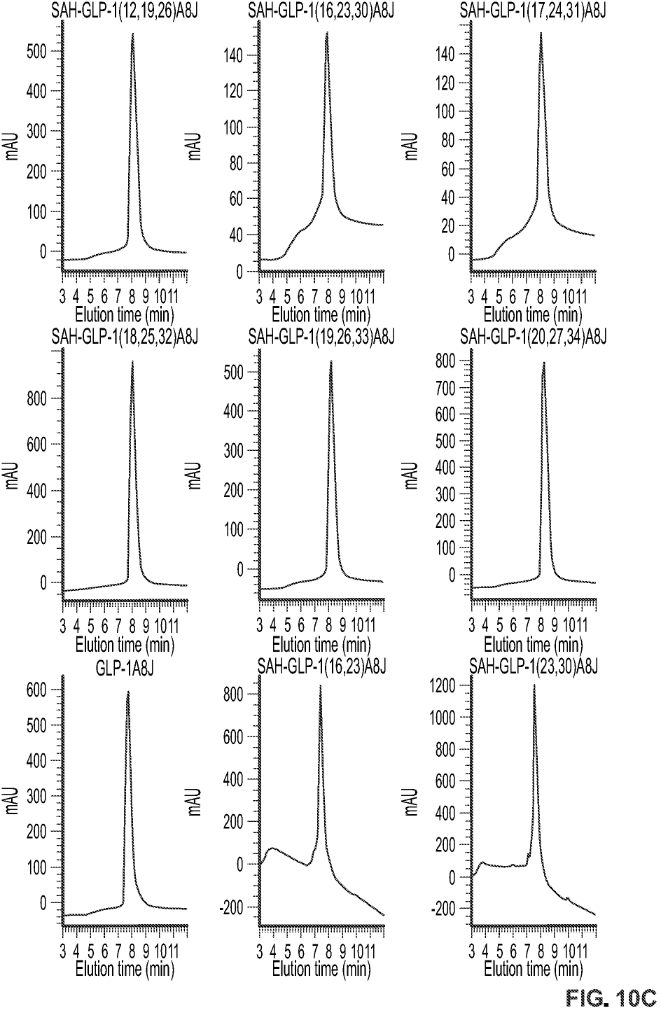

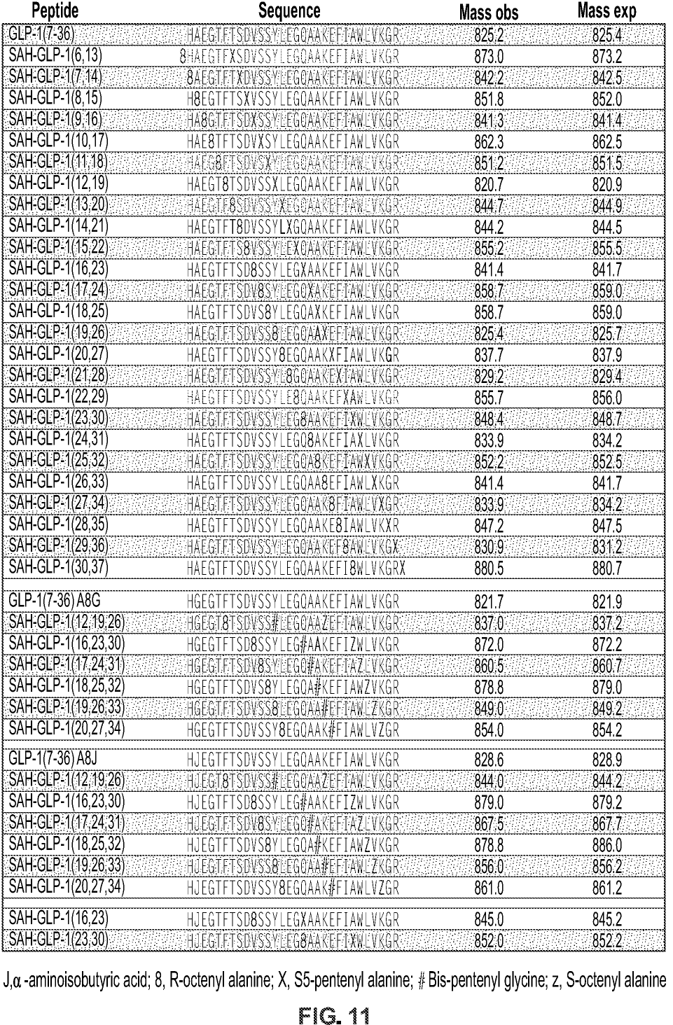

Peptide synthesis. All-hydrocarbon stapled peptides were synthesized on Rink Amide AM resin (Merck) with a free N terminus and purified to >95% homogeneity by LC/MS as previously described (Bird et al., Nature Chemical Biology. 2016;12(10):845-52). The i, i+7 staple scan was synthesized using (R)-N-Fmoc-α-(7′-octenyl)alanine and (S)-N-Fmoc-α-(4′-pentenyl)alanine at the N- and C-terminal staple positions, respectively. The stitched peptides were synthesized using, from N- to C-terminal staple positions, (S)-N-Fmoc-α-(7′-octenyl)alanine, N-Fmoc-α,α-Bis(4′-pentenyl)glycine, and (R)-N-Fmoc-α-(7′-octenyl)alanine (Nagase). HPLC profiles and masses of the generated compounds are presented in FIGS. 10A-10C and FIG. 11, respectively.

Cell culture. U2OS cells were stably reconstituted with human GLP-1 receptor (GenBank Acc. NM_002062) fused to the N-terminus of enhanced green fluorescent protein (EGFP) and continuous expression maintained by treatment with 0.5 mg/mL G418 in DMEM supplemented with 2 mM L-Glutamine, 1% penicillin-streptomycin, and 10% FBS. Cells were verified to be mycoplasma-free using the MycoAlert mycoplasma detection kit (Lonza Biologics Inc).

High content receptor internalization assay. For high-content epifluorescence microscopy analysis, cells were plated in black, clear-bottom 384-well plates overnight at a density of 4 × 10³ cells per well. The following day, the cells were exposed to fresh media containing Hoechst 33342 (1:5000 dilution of 10 mg/mL) and treated with various doses of peptides (e.g., FIG. 3: 10, 2.5, or 0.625 µM; FIG. 6A: serial dilution from 5 to 0.15 µM) at 37° C. for 1 hour followed by ImageXpress Microscopy imaging. For each treatment condition, performed in technical quadruplicate, data were collected at one central site per well at 10x magnification, followed by analysis and quantitation for internalized receptor foci using MetaXpress software. For each comparative analysis, all stapled peptides in the panel were measured on the same day using the same plating of cells and peptide dilutions. The entire experiment was then repeated at least twice more on different days using freshly plated cells and peptide dilutions.

cAMP Assay. Cyclic AMP production was measured using the cAMP Hunter eXpress GLP1R CHO-K1 GPCR Assay according to the manufacturer’s instructions (Eurofins, 95-0062E2CP2S). Briefly, the frozen cells were thawed and plated in 96 well format for overnight incubation at 37° C. in a humidified incubator, with the top two rows of the plate reserved for the cAMP standard. To generate the standard curve, the cAMP standard was diluted to achieve an initial concentration of 2.3 µM and then serially diluted 1:3 until reaching a final dose of 39 pM. SAH-GLP-1(16,23,30) A8J was diluted to achieve a starting concentration of 3.7 nM and serially diluted 1:3 to reach a final dose of 0.56 pM. The generated SAH-GLP-1(16,23,30) A8J dilutions were then added to the plated cells and allowed to incubate at 37° C. for 30 minutes. After workup with lysis buffer and cAMP antibody incubation per the manufacturer’s protocol, luminescence was read on a SpectraMax M5 microplate reader (Molecular Devices) at equilibrium. Nonlinear regression analysis was performed using Prism software (GraphPad) to obtain EC50s for the cAMP standard curve and cAMP induction by SAH-GLP-1-A8J(16,23,30).

Circular Dichroism Spectroscopy. Peptides were dissolved in 25% acetonitrile/water to achieve a concentration of 50 µM. Circular dichroism (CD) spectra were obtained on a spectropolarimeter (Aviv) using standard measurement parameters of temperature, 25° C.; wavelength, 190-260 nm; step resolution, 0.5 nm; speed, 20 nm min-1; accumulations, 10.

Hydrogen Deuterium Exchange Mass Spectrometry. For analysis of exchange into the indicated GLP-1 constructs, the peptides were dissolved in 25% acetonitrile/water at 50 µM and kept on ice. Deuterium labeling was initiated with an 18-fold dilution into a D₂O buffer (10 mM potassium phosphate pD 7.01, 100 mM NaCl) at 21° C. After 10 sec of labeling, the reaction was quenched with the addition of an equal volume of quenching buffer (150 mM sodium phosphate pH 2.48) at 0° C. Samples were then injected onto an in-house packed POROS 20-R2 trap for peptide trapping and desalting for 3 minutes. A Waters nanoACQUITY LC was used to elute each peptide from the trap with a 15%-70% gradient of acetonitrile over 6 minutes at a flow rate of 100 µL/min. Eluant was directed into a Waters Xevo G2 mass spectrometer operated in TOF-only mode for mass analysis. Data were analyzed as described (Barclay, L. A. et al. Mol. Cell 57, 873-886). All mass spectra were processed manually using MagTran. The relative amount of deuterium in the GLP-1 constructs was determined by subtracting the centroid mass of the undeuterated form from the deuterated form, at each condition. Deuterium levels were not corrected for back exchange and thus reported as relative.

Peptide proteolysis assay. Invitro proteolytic degradation was measured by LC/MS (Agilent 1200) using the following parameters: 20 µl injection, 0.6 mL flow rate, 20-min run time consisting of a gradient of water (0.1% formic acid) to 20%-80% acetonitrile (0.75% formic acid) over 15-min, 4-min wash to revert to starting gradient conditions, and 0.5-min post-time. The D AD signal was set to 280 nm with an 8-nm bandwidth and MSD set to scan mode with one channel at (M+2 H)/2, ± 1 mass units, and the other at (M+3 H)/3, ± 1 mass units. Integration of each MSD signal yielded areas under the curve of >108 counts. Reaction samples were composed of 5 µl peptide in DMSO (1 mM stock) and 195 µl buffer consisting of 50 mM Tris-HCl, pH 7.4.

Upon injection of the zero time sample, 2.5 µl of 100 ng/µL proteinase K (New England Biolabs) was added, and the amount of intact peptide quantitated by serial injection over time. A plot of MSD area versus time yielded an exponential decay curve, and half-lives were determined by nonlinear regression analysis using Prism software (GraphPad).

Plasma Stability Testing. Peptide stability was tested in freshly drawn mouse plasma collected in lithium heparin tubes. Peptide plasma incubations were set up with 500 µL of plasma spiked with 10 µM of the individual peptides. Samples were gently shaken in an orbital shaker at 37° C., and 20 µL aliquots were removed at 0, 5, 15, 30, 60, 120, and 200 minutes and added to 150 µL of a mixture containing 50% water/50% acetonitrile to stop further degradation of the peptides. The samples were allowed to sit on ice for the duration of the assay and then transferred to a MultiScreen Solvinert 0.45 µm low-binding hydrophilic PTFE plate (Millipore). The filtrate was directly analyzed by LC/MS/MS using a Thermo BetaSil column, 2.1 × 50 mm, 5 µm. The peptides were detected on a Sciex 6500 Qtrap mass spectrometer as +3 or +4 charged ions using the following mass transitions: 824.9 to 571.3 for GLP-1, 1029 to 690 for semaglutide, and 878.6 to 571.2 for SAH-GLP-1(16,23,30) A8J. The percentage of remaining peptide was determined by the decrease in chromatographic peak area and log transformed to calculate the half-life.

In vivo efficacy testing of SAH-GLP-1 peptides. Male B6 and db/db mice (JAX 000697) at 10-12 weeks of age were housed (5 mice/cage) at a constant room temperature of 24° C. on a normal day-light cycle and provided a standard diet ad libitum. After one week of acclimatization, B6 mice (n=8 per treatment arm) were fasted for 16 hours overnight and the following morning baseline blood glucose levels were measured (Onetouch), followed by intraperitoneal (IP) injection of vehicle (saline) or the indicated peptide (10 nmol/kg). Thirty minutes later, the mice were treated with a bolus IP injection of glucose (2 g/kg, 20% aqueous solution), followed by serial blood glucose monitoring at 0, 15, 30, 60, and 120 minutes. To evaluate the comparative anti-hyperglycemic effects of peptides in db/db mice (n=8 per treatment arm), vehicle (saline) or the indicated peptide (10 nmol/kg) was injected IP and blood glucose levels monitored at 0, 15, 30, 60, and 720 minutes.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A peptide comprising the amino acid sequence
(i) HJEGTFTSD8SSYLEG#AAKEFIZWLVKGR set forth in SEQ ID NO:40;
(ii) HGEGTFTSD8SSYLEG#AAKEFIZWLVKGR set forth in SEQ ID NO:33,
(iii) HJEGTFTSD8SSYLEG#AAKEFIZWLVKGRG set forth in SEQ ID NO:58;
(iv) HGEGTFTSD8SSYLEG#AAKEFIZWLVKGRG set forth in SEQ ID NO:57,
(v) HJEGTFTSDV8SYLEGQ#AKEFIAZLVKGR set forth in SEQ ID NO:41,
(vi) HGEGTFTSDV8SYLEGQ#AKEFIAZLVKGR set forth in SEQ ID NO:34,
(vii) HJEGTFTSDV8SYLEGQ#AKEFIAZLVKGRG set forth in SEQ ID NO:60, or
(viii) HGEGTFTSDV8SYLEGQ#AKEFIAZLVKGRG set forth in SEQ ID NO:59,
wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine, and J is 2-aminoisobutyric acid, and wherein the peptide binds to glucagon-like peptide 1 receptor (SEQ ID NO:5).

2. A stitched peptide comprising the amino acid sequence (i) HJEGTFTSD8SSYLEG#AAKEFIZWLVKGR set forth in SEQ ID NO:40; or (ii) HGEGTFTSD8SSYLEG#AAKEFIZWLVKGR set forth in SEQ ID NO:33, wherein 8 is (R)-α-(7′-octenyl)alanine or (R)-α-(4′-pentenyl)alanine, # is α,α-Bis(4′-pentenyl)glycine or α,α-Bis(7′-octenyl)glycine, and Z is (S)-α-(7′-octenyl)alanine or (S)-α-(4′-pentenyl)alanine, and J is 2-aminoisobutyric acid, and wherein the peptide binds to glucagon-like peptide 1 receptor (SEQ ID NO:5).

3. A stitched peptide comprising the amino acid sequence (i) HJEGTFTSDVSSYLEGQAAKEFIAWLVKGR set forth in SEQ ID NO:38, wherein J is 2-aminoisobutyric acid; or (ii) HGEGTFTSDVSSYLEGQAAKEFIAWLVKGR set forth in SEQ ID NO:31, wherein each of positions 10, 17, and 24 of SEQ ID NO:38 or 31 is replaced with a stapling amino acid, wherein a sidechain of the stapling amino acid at position 10 is cross-linked to a sidechain of the stapling amino acid at position 17 and a sidechain of the stapling amino acid at position 17 is cross-linked to a side chain of the stapling amino acid at position 24, and wherein the peptide binds to glucagon-like peptide 1 receptor (SEQ ID NO:5).

4. A stitched peptide comprising a stitched amino acid sequence having the formula: 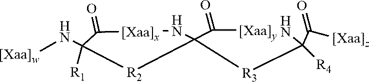
or a pharmaceutically acceptable salt thereof,
wherein:
[Xaa]_{w} is HJEGTFTSD, wherein J is 2-aminoisobutyric acid (SEQ ID NO:45) or
HGEGTFTSD (SEQ ID NO:49),
[Xaa]ₓ is SSYLEG (SEQ ID NO:46),
[Xaa]_{y} is AAKEFI (SEQ ID NO:47),
[Xaa]_{z} is WLVKGR (SEQ ID NO:48),
each R₁ and R₄ is independently H, alkyl, alkenyl, alkynyl, arylalkyl, cycloalkylalkyl, heteroarylalkyl, or heterocyclylalkyl, any of which is substituted or unsubstituted;
each R₂ and R₃ is independently alkylene, alkenylene, or alkynylene, any of which is substituted or unsubstituted;
wherein the stitched amino acid sequence binds to glucagon-like peptide 1 receptor (SEQ ID NO:5), and
wherein the stitched amino acid sequence has an alpha helical conformation.

5. The stitched peptide or pharmaceutically acceptable salt thereof of claim 4, wherein R₁ is an alkyl.

6. The stitched peptide or pharmaceutically acceptable salt thereof of claim 4, wherein R₁ is a methyl group.

7. The stitched peptide or pharmaceutically acceptable salt thereof of any one of claims 4 to 6, wherein R₄ is an alkyl.

8. The stitched peptide or pharmaceutically acceptable salt thereof of any one of claims 4 to 6, wherein R₄ is a methyl group.

9. The stitched peptide or pharmaceutically acceptable salt thereof of any one of claims 4 to 8, wherein R₂ is an alkenyl.

10. The stitched peptide or pharmaceutically acceptable salt thereof of any one of claims 4 to 9, wherein R₃ is an alkenyl.

11. The stitched peptide or pharmaceutically acceptable salt thereof of claim 3, wherein R₁ is a methyl group, R₂ is (CH₂)₆-CH=CH-(CH₂)₃, R₃ is (CH₂)₃-CH=CH-(CH₂)₆, and R₄ is a methyl group.

12. The stitched peptide or pharmaceutically acceptable salt thereof of claim 3, wherein R1 is a methyl group, R₂ is (CH₂)₃-CH=CH-(CH₂)₆, R₃ is (CH₂)₆-CH=CH-(CH₂)₃ and R4 is a methyl group.

13. The stitched peptide or pharmaceutically acceptable salt thereof of claim 4, wherein the stitched amino acid sequence comprises 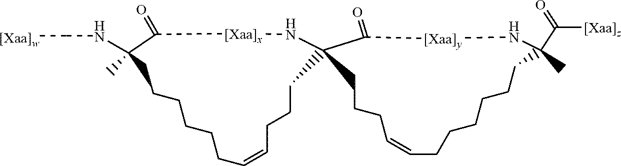
, wherein [Xaa]_{w} is HJEGTFTSD wherein J is 2-aminoisobutyric acid (SEQ ID NO:45) or HGEGTFTSD (SEQ ID NO:49), [Xaa]ₓ is SSYLEG (SEQ ID NO:46), [Xaa]y is AAKEFI (SEQ ID NO:47), and [Xaa]z is WLVKGR (SEQ ID NO:48).

14. The peptide of any one of claims 1 to 13, which is 30 to 50 amino acids in length.

15. A stitched peptide comprising a modified amino acid sequence of the sequence set forth in SEQ ID NO:38, wherein the peptide comprises a stitch between amino acids corresponding to positions 10, 17, and 24 of SEQ ID NO:38, and wherein the peptide binds to glucagon-like peptide 1 receptor (SEQ ID NO:5).

16. A pharmaceutical composition comprising the peptide of any one of claims 1 to 4 and 15 and a pharmaceutically acceptable carrier.

17. A method of treating diabetes in a human subject in need thereof, the method comprising administering a therapeutically-effective amount of the peptide of claim 1 to the subject.

18. A method of treating diabetes in a human subject in need thereof, the method comprising administering a therapeutically-effective amount of the stitched peptide of any one of claims 2 to 4 and 15 to the subject.

19. A method of treating hyperglycemia in a human subject in need thereof, the method comprising administering a therapeutically-effective amount of the peptide of claim 1 to the subject.

20. A method of treating hyperglycemia in a human subject in need thereof, the method comprising administering a therapeutically-effective amount of the stitched peptide of any one of claims 2 to 4 and 15 to the subject.

21. A method of treating rapid gastric emptying, insulin resistance, or cardiovascular disease in a human subject in need thereof, the method comprising administering a therapeutically-effective amount of the peptide of claim 1 to the subject.

22. A method of treating rapid gastric emptying, insulin resistance, or cardiovascular disease in a human subject in need thereof, the method comprising administering a therapeutically-effective amount of the stitched peptide of any one of claims 2 to 4 and 15 to the subject.

23. A method of treating Alzheimer’s disease in a human subject in need thereof, the method comprising administering a therapeutically-effective amount of the peptide of claim 1 to the subject.

24. A method of treating Alzheimer’s disease in a human subject in need thereof, the method comprising administering a therapeutically-effective amount of the stitched peptide of any one of claims 2 to 4 and 15 to the subject.

25. A method of treating Huntington’s disease in a human subject in need thereof, the method comprising administering a therapeutically-effective amount of the peptide of claim 1 to the subject.

26. A method of treating Huntington’s disease in a human subject in need thereof, the method comprising administering a therapeutically-effective amount of the stitched peptide of any one of claims 2 to 4 and 15 to the subject.

27. A method of making a stitched peptide, the method comprising: (a) providing a peptide having the sequence set forth in SEQ ID NO:61, 40, 62, or 33, and (b) cross-linking the peptide.

28. A peptide comprising the amino acid sequence of any one of SEQ ID NOs: 61, 62, 65, 66, 71, 73, 79, 81, 67, 68, 75, 77, 83, and 85, wherein the peptide binds to glucagon-like peptide 1 receptor (SEQ ID NO:5).

29. A stitched peptide comprising the amino acid sequence of any one of SEQ ID NOs: 34, 41, 59-68, 71, 73, 75, 77, 79, 81, 83, and 85, wherein the stitched peptide binds to glucagon-like peptide 1 receptor (SEQ ID NO:5).

30. A stitched peptide comprising the amino acid sequence
(i) HJEGTFTSDVSSYLEGQAAKEFIA WL VKGR set forth in SEQ ID NO:38, wherein J is 2-aminoisobutyric acid; or
(ii) HGEGTFTSDVSSYLEGQAAKEFIAWLVKGR set forth in SEQ ID NO:31,
wherein
(a) each of positions 11, 18, and 25 of SEQ ID NO:38 or 31 is replaced with a stapling amino acid, wherein a sidechain of the stapling amino acid at position 11 is cross-linked to a sidechain of the stapling amino acid at position 18 and a sidechain of the stapling amino acid at position 18 is cross-linked to a side chain of the stapling amino acid at position 25,
(b) each of positions 12, 19, and 26 of SEQ ID NO:38 or 31 is replaced with a stapling amino acid, wherein a sidechain of the stapling amino acid at position 12 is cross-linked to a sidechain of the stapling amino acid at position 19 and a sidechain of the stapling amino acid at position 19 is cross-linked to a side chain of the stapling amino acid at position 26, or
(c) each of positions 6, 13, and 20 of SEQ ID NO:38 or 31 is replaced with a stapling amino acid, wherein a sidechain of the stapling amino acid at position 6 is cross-linked to a sidechain of the stapling amino acid at position 13 and a sidechain of the stapling amino acid at position 13 is cross-linked to a side chain of the stapling amino acid at position 20,
and
wherein the stitched peptide binds to glucagon-like peptide 1 receptor (SEQ ID NO:5).

31. A stitched peptide comprising a stitched amino acid sequence having the formula: 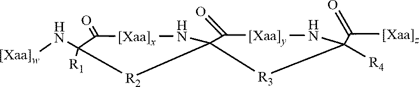
or a pharmaceutically acceptable salt thereof,
wherein:
(a) [Xaa]_{w} HJEGTFTSDV (SEQ ID NO:50) or HGEGTFTSDV (SEQ ID NO:54),
[Xaa]ₓ is SYLEGQ (SEQ ID NO:51),
[Xaa]_{y} is AKEFIA (SEQ ID NO:52),
[Xaa]_{z} is LVKGR (SEQ ID NO:53) or LVKGRG (SEQ ID NO:56),
(b) [Xaa]_{w} HGEGTFTSDVS (SEQ ID NO:95) or HJEGTFTSDVS (SEQ ID NO:96),
[Xaa]ₓ is YLEGQA (SEQ ID NO:89),
[Xaa]_{y} is KEFIAW (SEQ ID NO:90),
[Xaa]_{z} is VKGR (SEQ ID NO:97) or VKGRG (SEQ ID NO:98), or
(c) [Xaa]_{w} is HGEGT (SEQ ID NO:91) or HJEGT (SEQ ID NO:92),
[Xaa]ₓ is TSDVSS (SEQ ID NO:87),
[Xaa]_{y} is LEGQAA (SEQ ID NO:88),
[Xaa]_{z} is EFIAWLVKGR (SEQ ID NO:93) or EFIAWLVKGRG (SEQ ID NO:94),
each R₁ and R₄ is independently H, alkyl, alkenyl, alkynyl, arylalkyl, cycloalkylalkyl, heteroarylalkyl, or heterocyclylalkyl, any of which is substituted or unsubstituted;
each R₂ and R₃ is independently alkylene, alkenylene, or alkynylene, any of which is substituted or unsubstituted;
wherein J is 2-aminoisobutryic acid,
wherein the stitched amino acid sequence binds to glucagon-like peptide 1 receptor (SEQ ID NO:5), and
wherein the stitched amino acid sequence has an alpha helical conformation.

32. A stitched peptide comprising a modified amino acid sequence of the sequence set forth in SEQ ID NO:38 or 31, wherein the peptide comprises a stitch between amino acids corresponding to (a) positions 11, 18, and 25 of SEQ ID NO:38 or 31, (b) positions 12, 19, and 26 of SEQ ID NO:38 or 31, or (c) 6, 13, and 20, and wherein the stitched peptide binds to glucagon-like peptide 1 receptor (SEQ ID NO:5).

33. A pharmaceutical composition comprising the peptide of claim 28 or the stitched peptide of any one of claims 29 to 32 and a pharmaceutically acceptable carrier.

34. A method of treating diabetes in a human subject in need thereof, the method comprising administering a therapeutically-effective amount of the peptide of claim 28 or the stitched peptide of any one of claims 29 to 32 to the human subject.

35. A method of treating hyperglycemia in a human subject in need thereof, the method comprising administering a therapeutically-effective amount of the peptide of claim 28 or the stitched peptide of any one of claims 29 to 32 to the human subject.

36. A method of treating rapid gastric emptying, insulin resistance, or cardiovascular disease in a human subject in need thereof, the method comprising administering a therapeutically-effective amount of the peptide of claim 28 or the stitched peptide of any one of claims 29 to 32 to the human subject.

37. A method of treating Alzheimer’s disease in a human subject in need thereof, the method comprising administering a therapeutically-effective amount of the peptide of claim 28 or the stitched peptide of any one of claims 29 to 32 to the human subject.

38. A method of treating Huntington’s disease in a human subject in need thereof, the method comprising administering a therapeutically-effective amount of the peptide of claim 28 or the stitched peptide of any one of claims 29 to 32 to the human subject.

39. A method of increasing cAMP levels in a human subject in need thereof, the method comprising administering a therapeutically-effective amount of the peptide of claim 1 or 28 or the stitched peptide of any one of claims 2 to 28 and 29 to 32 to the human subject.

40. A method of making a stitched peptide, the method comprising: (a) providing a peptide having the sequence set forth in SEQ ID NO: 34, 41, 59-68, 71, 73, 75, 77, 79, 81, 83, and 85, and (b) cross-linking the peptide.

41. A pharmaceutical composition comprising:
(a) a means for treating diabetes, hyperglycemia, rapid gastric emptying, insulin resistance, cardiovascular disease, Alzheimer’s disease, or Huntington’s disease, and
(b) a pharmaceutically acceptable carrier.

42. The pharmaceutical composition of claim 41, wherein the means for treating diabetes, hyperglycemia, rapid gastric emptying, insulin resistance, cardiovascular disease, Alzheimer’s disease, or Huntington’s disease are stitched GLP-1 peptides.

43. A pharmaceutical composition comprising:
(a) a means for increasing cAMP levels, and
(b) a pharmaceutically acceptable carrier.

44. The pharmaceutical composition of claim 43, wherein the means for increasing cAMP levels are stitched GLP-1 peptides.

45. A pharmaceutical composition comprising:
(a) a means for binding and agonizing GLP-1 reeceptor, and
(b) a pharmaceutically acceptable carrier.

46. The pharmaceutical composition of claim 43, wherein the means for for binding and agonizing GLP-1 receptor are stitched GLP-1 peptides.


SEQUENCE LISTING (110 sequences)

SEQ ID NO: 1 - protein, 179 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDR

SEQ ID NO: 2 - protein, 37 aa, Homo sapiens
HDEFERHAEGTFTSDVSSYLEGQAAKEFIAWLVKGRG

SEQ ID NO: 3 - protein, 31 aa, Homo sapiens
HAEGTFTSDVSSYLEGQAAKEFIAWLVKGRG

SEQ ID NO: 4 - protein, 30 aa, Homo sapiens
HAEGTFTSDVSSYLEGQAAKEFIAWLVKGR

SEQ ID NO: 5 - protein, 440 aa, Homo sapiens
RPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCS

SEQ ID NO: 6 - protein, 31 aa, Artificial Sequence
  source: /note="Description of Artificial Sequence: Synthetic polypeptide"
  MOD_RES (1)..(1): (R)-alpha-(7'-octenyl)alanine
  MOD_RES (8)..(8): (S)-alpha-(4'-pentenyl)alanine
  source: /note="See specification as filed for detailed description of substitutions and preferred embodiments"
XHAEGTFXSDVSSYLEGQAAKEFIAWLVKGR

SEQ ID NO: 7 - protein, 30 aa, Artificial Sequence
  source: /note="Description of Artificial Sequence: Synthetic polypeptide"
  MOD_RES (1)..(1): (R)-alpha-(7'-octenyl)alanine
  MOD_RES (8)..(8): (S)-alpha-(4'-pentenyl)alanine
  source: /note="See specification as filed for detailed description of substitutions and preferred embodiments"
XAEGTFTXDVSSYLEGQAAKEFIAWLVKGR

SEQ ID NO: 8 - protein, 30 aa, Artificial Sequence
  source: /note="Description of Artificial Sequence: Synthetic polypeptide"
  MOD_RES (2)..(2): (R)-alpha-(7'-octenyl)alanine
  MOD_RES (9)..(9): (S)-alpha-(4'-pentenyl)alanine
  source: /note="See specification as filed for detailed description of substitutions and preferred embodiments"
HXEGTFTSXVSSYLEGQAAKEFIAWLVKGR

SEQ ID NO: 9 - protein, 30 aa, Artificial Sequence
  source: /note="Description of Artificial Sequence: Synthetic polypeptide"
  MOD_RES (3)..(3): (R)-alpha-(7'-octenyl)alanine
  MOD_RES (10)..(10): (S)-alpha-(4'-pentenyl)alanine
  source: /note="See specification as filed for detailed description of substitutions and preferred embodiments"
HAXGTFTSDXSSYLEGQAAKEFIAWLVKGR

SEQ ID NO: 10 - protein, 30 aa, Artificial Sequence
  source: /note="Description of Artificial Sequence: Synthetic polypeptide"
  MOD_RES (4)..(4): (R)-alpha-(7'-octenyl)alanine
  MOD_RES (11)..(11): (S)-alpha-(4'-pentenyl)alanine
  source: /note="See specification as filed for detailed description of substitutions and preferred embodiments"
HAEXTFTSDVXSYLEGQAAKEFIAWLVKGR

SEQ ID NO: 11 - protein, 30 aa, Artificial Sequence
  source: /note="Description of Artificial Sequence: Synthetic polypeptide"
  MOD_RES (5)..(5): (R)-alpha-(7'-octenyl)alanine
  MOD_RES (12)..(12): (S)-alpha-(4'-pentenyl)alanine
  source: /note="See specification as filed for detailed description of substitutions and preferred embodiments"
HAEGXFTSDVSXYLEGQAAKEFIAWLVKGR

SEQ ID NO: 12 - protein, 30 aa, Artificial Sequence
  source: /note="Description of Artificial Sequence: Synthetic polypeptide"
  MOD_RES (6)..(6): (R)-alpha-(7'-octenyl)alanine
  MOD_RES (13)..(13): (S)-alpha-(4'-pentenyl)alanine
  source: /note="See specification as filed for detailed description of substitutions and preferred embodiments"
HAEGTXTSDVSSXLEGQAAKEFIAWLVKGR

SEQ ID NO: 13 - protein, 30 aa, Artificial Sequence
  source: /note="Description of Artificial Sequence: Synthetic polypeptide"
  MOD_RES (7)..(7): (R)-alpha-(7'-octenyl)alanine
  MOD_RES (14)..(14): (S)-alpha-(4'-pentenyl)alanine
  source: /note="See specification as filed for detailed description of substitutions and preferred embodiments"
HAEGTFXSDVSSYXEGQAAKEFIAWLVKGR

SEQ ID NO: 14 - protein, 30 aa, Artificial Sequence
  source: /note="Description of Artificial Sequence: Synthetic polypeptide"
  MOD_RES (8)..(8): (R)-alpha-(7'-octenyl)alanine
  MOD_RES (15)..(15): (S)-alpha-(4'-pentenyl)alanine
  source: /note="See specification as filed for detailed description of substitutions and preferred embodiments"
HAEGTFTXDVSSYLXGQAAKEFIAWLVKGR

SEQ ID NO: 15 - protein, 30 aa, Artificial Sequence
  source: /note="Description of Artificial Sequence: Synthetic polypeptide"
  MOD_RES (9)..(9): (R)-alpha-(7'-octenyl)alanine
  MOD_RES (16)..(16): (S)-alpha-(4'-pentenyl)alanine
  source: /note="See specification as filed for detailed description of substitutions and preferred embodiments"
HAEGTFTSXVSSYLEXQAAKEFIAWLVKGR

SEQ ID NO: 16 - protein, 30 aa, Artificial Sequence
  source: /note="Description of Artificial Sequence: Synthetic polypeptide"
  MOD_RES (10)..(10): (R)-alpha-(7'-octenyl)alanine
  MOD_RES (17)..(17): (S)-alpha-(4'-pentenyl)alanine
  source: /note="See specification as filed for detailed description of substitutions and preferred embodiments"
HAEGTFTSDXSSYLEGXAAKEFIAWLVKGR

SEQ ID NO: 17 - protein, 30 aa, Artificial Sequence
  source: /note="Description of Artificial Sequence: Synthetic polypeptide"
  MOD_RES (11)..(11): (R)-alpha-(7'-octenyl)alanine
  MOD_RES (18)..(18): (S)-alpha-(4'-pentenyl)alanine
  source: /note="See specification as filed for detailed description of substitutions and preferred embodiments"
HAEGTFTSDVXSYLEGQXAKEFIAWLVKGR

SEQ ID NO: 18 - protein, 30 aa, Artificial Sequence
  source: /note="Description of Artificial Sequence: Synthetic polypeptide"
  MOD_RES (12)..(12): (R)-alpha-(7'-octenyl)alanine
  MOD_RES (19)..(19): (S)-alpha-(4'-pentenyl)alanine
  source: /note="See specification as filed for detailed description of substitutions and preferred embodiments"
HAEGTFTSDVSXYLEGQAXKEFIAWLVKGR

SEQ ID NO: 19 - protein, 30 aa, Artificial Sequence
  source: /note="Description of Artificial Sequence: Synthetic polypeptide"
  MOD_RES (13)..(13): (R)-alpha-(7'-octenyl)alanine
  MOD_RES (20)..(20): (S)-alpha-(4'-pentenyl)alanine
  source: /note="See specification as filed for detailed description of substitutions and preferred embodiments"
HAEGTFTSDVSSXLEGQAAXEFIAWLVKGR

SEQ ID NO: 20 - protein, 30 aa, Artificial Sequence
  source: /note="Description of Artificial Sequence: Synthetic polypeptide"
  MOD_RES (14)..(14): (R)-alpha-(7'-octenyl)alanine
  MOD_RES (21)..(21): (S)-alpha-(4'-pentenyl)alanine
  source: /note="See specification as filed for detailed description of substitutions and preferred embodiments"
HAEGTFTSDVSSYXEGQAAKXFIAWLVKGR

SEQ ID NO: 21 - protein, 30 aa, Artificial Sequence
  source: /note="Description of Artificial Sequence: Synthetic polypeptide"
  MOD_RES (15)..(15): (R)-alpha-(7'-octenyl)alanine
  MOD_RES (22)..(22): (S)-alpha-(4'-pentenyl)alanine
  source: /note="See specification as filed for detailed description of substitutions and preferred embodiments"
HAEGTFTSDVSSYLXGQAAKEXIAWLVKGR

SEQ ID NO: 22 - protein, 30 aa, Artificial Sequence
  source: /note="Description of Artificial Sequence: Synthetic polypeptide"
  MOD_RES (16)..(16): (R)-alpha-(7'-octenyl)alanine
  MOD_RES (23)..(23): (S)-alpha-(4'-pentenyl)alanine
  source: /note="See specification as filed for detailed description of substitutions and preferred embodiments"
HAEGTFTSDVSSYLEXQAAKEFXAWLVKGR

SEQ ID NO: 23 - protein, 30 aa, Artificial Sequence
  source: /note="Description of Artificial Sequence: Synthetic polypeptide"
  MOD_RES (17)..(17): (R)-alpha-(7'-octenyl)alanine
  MOD_RES (24)..(24): (S)-alpha-(4'-pentenyl)alanine
  source: /note="See specification as filed for detailed description of substitutions and preferred embodiments"
HAEGTFTSDVSSYLEGXAAKEFIXWLVKGR

SEQ ID NO: 24 - protein, 30 aa, Artificial Sequence
  source: /note="Description of Artificial Sequence: Synthetic polypeptide"
  MOD_RES (18)..(18): (R)-alpha-(7'-octenyl)alanine
  MOD_RES (25)..(25): (S)-alpha-(4'-pentenyl)alanine
  source: /note="See specification as filed for detailed description of substitutions and preferred embodiments"
HAEGTFTSDVSSYLEGQXAKEFIAXLVKGR

SEQ ID NO: 25 - protein, 30 aa, Artificial Sequence
  source: /note="Description of Artificial Sequence: Synthetic polypeptide"
  MOD_RES (19)..(19): (R)-alpha-(7'-octenyl)alanine
  MOD_RES (26)..(26): (S)-alpha-(4'-pentenyl)alanine
  source: /note="See specification as filed for detailed description of substitutions and preferred embodiments"
HAEGTFTSDVSSYLEGQAXKEFIAWXVKGR

SEQ ID NO: 26 - protein, 30 aa, Artificial Sequence
  source: /note="Description of Artificial Sequence: Synthetic polypeptide"
  MOD_RES (20)..(20): (R)-alpha-(7'-octenyl)alanine
  MOD_RES (27)..(27): (S)-alpha-(4'-pentenyl)alanine
  source: /note="See specification as filed for detailed description of substitutions and preferred embodiments"
HAEGTFTSDVSSYLEGQAAXEFIAWLXKGR

SEQ ID NO: 27 - protein, 30 aa, Artificial Sequence
  source: /note="Description of Artificial Sequence: Synthetic polypeptide"
  MOD_RES (21)..(21): (R)-alpha-(7'-octenyl)alanine
  MOD_RES (28)..(28): (S)-alpha-(4'-pentenyl)alanine
  source: /note="See specification as filed for detailed description of substitutions and preferred embodiments"
HAEGTFTSDVSSYLEGQAAKXFIAWLVXGR

SEQ ID NO: 28 - protein, 30 aa, Artificial Sequence
  source: /note="Description of Artificial Sequence: Synthetic polypeptide"
  MOD_RES (22)..(22): (R)-alpha-(7'-octenyl)alanine
  MOD_RES (29)..(29): (S)-alpha-(4'-pentenyl)alanine
  source: /note="See specification as filed for detailed description of substitutions and preferred embodiments"
HAEGTFTSDVSSYLEGQAAKEXIAWLVKXR

SEQ ID NO: 29 - protein, 30 aa, Artificial Sequence
  source: /note="Description of Artificial Sequence: Synthetic polypeptide"
  MOD_RES (23)..(23): (R)-alpha-(7'-octenyl)alanine
  MOD_RES (30)..(30): (S)-alpha-(4'-pentenyl)alanine
  source: /note="See specification as filed for detailed description of substitutions and preferred embodiments"
HAEGTFTSDVSSYLEGQAAKEFXAWLVKGX

SEQ ID NO: 30 - protein, 31 aa, Artificial Sequence
  source: /note="Description of Artificial Sequence: Synthetic polypeptide"
  MOD_RES (24)..(24): (R)-alpha-(7'-octenyl)alanine
  MOD_RES (31)..(31): (S)-alpha-(4'-pentenyl)alanine
  source: /note="See specification as filed for detailed description of substitutions and preferred embodiments"
HAEGTFTSDVSSYLEGQAAKEFIXWLVKGRX

SEQ ID NO: 31 - protein, 30 aa, Artificial Sequence
  source: /note="Description of Artificial Sequence: Synthetic polypeptide"
HGEGTFTSDVSSYLEGQAAKEFIAWLVKGR

SEQ ID NO: 32 - protein, 30 aa, Artificial Sequence
  source: /note="Description of Artificial Sequence: Synthetic polypeptide"
  MOD_RES (6)..(6): (R)-alpha-(7'-octenyl)alanine
  MOD_RES (13)..(13): Alpha,alpha-Bis(4'-pentenyl)glycine
  MOD_RES (20)..(20): (S)-alpha-(7'-octenyl)alanine
  source: /note="See specification as filed for detailed description of substitutions and preferred embodiments"
HGEGTXTSDVSSXLEGQAAXEFIAWLVKGR

SEQ ID NO: 33 - protein, 30 aa, Artificial Sequence
  source: /note="Description of Artificial Sequence: Synthetic polypeptide"
  MOD_RES (10)..(10): (R)-alpha-(7'-octenyl)alanine or (R)-alpha-(4'-pentenyl)alanine
  MOD_RES (17)..(17): alpha,alpha-Bis(4'-pentenyl)glycine or alpha,alpha-Bis (7'-octenyl)glycine
  MOD_RES (24)..(24): (S)-alpha-(7'-octenyl)alanine or (S)-alpha-(4'-pentenyl)alanine
  source: /note="See specification as filed for detailed description of substitutions and preferred embodiments"
HGEGTFTSDXSSYLEGXAAKEFIXWLVKGR

SEQ ID NO: 34 - protein, 30 aa, Artificial Sequence
  source: /note="Description of Artificial Sequence: Synthetic polypeptide"
  MOD_RES (11)..(11): (R)-alpha-(7'-octenyl)alanine or (R)-alpha-(4'-pentenyl)alanine
  MOD_RES (18)..(18): alpha,alpha-Bis(4'-pentenyl)glycine or alpha,alpha-Bis (7'-octenyl)glycine
  MOD_RES (25)..(25): (S)-alpha-(7'-octenyl)alanine or (S)-alpha-(4'-pentenyl)alanine
  source: /note="See specification as filed for detailed description of substitutions and preferred embodiments"
HGEGTFTSDVXSYLEGQXAKEFIAXLVKGR

SEQ ID NO: 35 - protein, 30 aa, Artificial Sequence
  source: /note="Description of Artificial Sequence: Synthetic polypeptide"
  MOD_RES (12)..(12): (R)-alpha-(7'-octenyl)alanine
  MOD_RES (19)..(19): Alpha,alpha-Bis(4'-pentenyl)glycine
  MOD_RES (26)..(26): (S)-alpha-(7'-octenyl)alanine
  source: /note="See specification as filed for detailed description of substitutions and preferred embodiments"
HGEGTFTSDVSXYLEGQAXKEFIAWXVKGR

SEQ ID NO: 36 - protein, 30 aa, Artificial Sequence
  source: /note="Description of Artificial Sequence: Synthetic polypeptide"
  MOD_RES (13)..(13): (R)-alpha-(7'-octenyl)alanine
  MOD_RES (20)..(20): Alpha,alpha-Bis(4'-pentenyl)glycine
  MOD_RES (27)..(27): (S)-alpha-(7'-octenyl)alanine
  source: /note="See specification as filed for detailed description of substitutions and preferred embodiments"
HGEGTFTSDVSSXLEGQAAXEFIAWLXKGR

SEQ ID NO: 37 - protein, 30 aa, Artificial Sequence
  source: /note="Description of Artificial Sequence: Synthetic polypeptide"
  MOD_RES (14)..(14): (R)-alpha-(7'-octenyl)alanine
  MOD_RES (21)..(21): Alpha,alpha-Bis(4'-pentenyl)glycine
  MOD_RES (28)..(28): (S)-alpha-(7'-octenyl)alanine
  source: /note="See specification as filed for detailed description of substitutions and preferred embodiments"
HGEGTFTSDVSSYXEGQAAKXFIAWLVXGR

SEQ ID NO: 38 - protein, 30 aa, Artificial Sequence
  source: /note="Description of Artificial Sequence: Synthetic polypeptide"
  MOD_RES (2)..(2): 2-aminoisobutyric acid
  source: /note="See specification as filed for detailed description of substitutions and preferred embodiments"
HXEGTFTSDVSSYLEGQAAKEFIAWLVKGR

SEQ ID NO: 39 - protein, 30 aa, Artificial Sequence
  source: /note="Description of Artificial Sequence: Synthetic polypeptide"
  MOD_RES (2)..(2): 2-aminoisobutyric acid
  MOD_RES (6)..(6): (R)-alpha-(7'-octenyl)alanine
  MOD_RES (13)..(13): Alpha,alpha-Bis(4'-pentenyl)glycine
  MOD_RES (20)..(20): (S)-alpha-(7'-octenyl)alanine
  source: /note="See specification as filed for detailed description of substitutions and preferred embodiments"
HXEGTXTSDVSSXLEGQAAXEFIAWLVKGR

SEQ ID NO: 40 - protein, 30 aa, Artificial Sequence
  source: /note="Description of Artificial Sequence: Synthetic polypeptide"
  MOD_RES (2)..(2): 2-aminoisobutyric acid
  MOD_RES (10)..(10): (R)-alpha-(7'-octenyl)alanine or (R)-alpha-(4'-pentenyl)alanine
  MOD_RES (17)..(17): alpha,alpha-Bis(4'-pentenyl)glycine or alpha,alpha-Bis (7'-octenyl)glycine
  MOD_RES (24)..(24): (S)-alpha-(7'-octenyl)alanine or (S)-alpha-(4'-pentenyl)alanine
  source: /note="See specification as filed for detailed description of substitutions and preferred embodiments"
HXEGTFTSDXSSYLEGXAAKEFIXWLVKGR

SEQ ID NO: 41 - protein, 30 aa, Artificial Sequence
  source: /note="Description of Artificial Sequence: Synthetic polypeptide"
  MOD_RES (2)..(2): 2-aminoisobutyric acid
  MOD_RES (11)..(11): (R)-alpha-(7'-octenyl)alanine or (R)-alpha-(4'-pentenyl)alanine
  MOD_RES (18)..(18): alpha,alpha-Bis(4'-pentenyl)glycine or alpha,alpha-Bis (7'-octenyl)glycine
  MOD_RES (25)..(25): (S)-alpha-(7'-octenyl)alanine or (S)-alpha-(4'-pentenyl)alanine
  source: /note="See specification as filed for detailed description of substitutions and preferred embodiments"
HXEGTFTSDVXSYLEGQXAKEFIAXLVKGR

SEQ ID NO: 42 - protein, 30 aa, Artificial Sequence
  source: /note="Description of Artificial Sequence: Synthetic polypeptide"
  MOD_RES (2)..(2): 2-aminoisobutyric acid
  MOD_RES (12)..(12): (R)-alpha-(7'-octenyl)alanine
  MOD_RES (19)..(19): Alpha,alpha-Bis(4'-pentenyl)glycine
  MOD_RES (26)..(26): (S)-alpha-(7'-octenyl)alanine
  source: /note="See specification as filed for detailed description of substitutions and preferred embodiments"
HXEGTFTSDVSXYLEGQAXKEFIAWXVKGR

SEQ ID NO: 43 - protein, 30 aa, Artificial Sequence
  source: /note="Description of Artificial Sequence: Synthetic polypeptide"
  MOD_RES (2)..(2): 2-aminoisobutyric acid
  MOD_RES (13)..(13): (R)-alpha-(7'-octenyl)alanine
  MOD_RES (20)..(20): Alpha,alpha-Bis(4'-pentenyl)glycine
  MOD_RES (27)..(27): (S)-alpha-(7'-octenyl)alanine
  source: /note="See specification as filed for detailed description of substitutions and preferred embodiments"
HXEGTFTSDVSSXLEGQAAXEFIAWLXKGR

SEQ ID NO: 44 - protein, 30 aa, Artificial Sequence
  source: /note="Description of Artificial Sequence: Synthetic polypeptide"
  MOD_RES (2)..(2): 2-aminoisobutyric acid
  MOD_RES (14)..(14): (R)-alpha-(7'-octenyl)alanine
  MOD_RES (21)..(21): Alpha,alpha-Bis(4'-pentenyl)glycine
  MOD_RES (28)..(28): (S)-alpha-(7'-octenyl)alanine
  source: /note="See specification as filed for detailed description of substitutions and preferred embodiments"
HXEGTFTSDVSSYXEGQAAKXFIAWLVXGR

SEQ ID NO: 45 - protein, 9 aa, Artificial Sequence
  source: /note="Description of Artificial Sequence: Synthetic peptide"
  MOD_RES (2)..(2): 2-aminoisobutyric acid
HXEGTFTSD

SEQ ID NO: 46 - protein, 6 aa, Artificial Sequence
  source: /note="Description of Artificial Sequence: Synthetic peptide"
SSYLEG

SEQ ID NO: 47 - protein, 6 aa, Artificial Sequence
  source: /note="Description of Artificial Sequence: Synthetic peptide"
AAKEFI

SEQ ID NO: 48 - protein, 6 aa, Artificial Sequence
  source: /note="Description of Artificial Sequence: Synthetic peptide"
WLVKGR

SEQ ID NO: 49 - protein, 9 aa, Artificial Sequence
  source: /note="Description of Artificial Sequence: Synthetic peptide"
HGEGTFTSD

SEQ ID NO: 50 - protein, 10 aa, Artificial Sequence
  source: /note="Description of Artificial Sequence: Synthetic peptide"
  MOD_RES (2)..(2): 2-aminoisobutyric acid
HXEGTFTSDV

SEQ ID NO: 51 - protein, 6 aa, Artificial Sequence
  source: /note="Description of Artificial Sequence: Synthetic peptide"
SYLEGQ

SEQ ID NO: 52 - protein, 6 aa, Artificial Sequence
  source: /note="Description of Artificial Sequence: Synthetic peptide"
AKEFIA

SEQ ID NO: 53 - protein, 5 aa, Artificial Sequence
  source: /note="Description of Artificial Sequence: Synthetic peptide"
LVKGR

SEQ ID NO: 54 - protein, 10 aa, Artificial Sequence
  source: /note="Description of Artificial Sequence: Synthetic peptide"
HGEGTFTSDV

SEQ ID NO: 55 - protein, 7 aa, Artificial Sequence
  source: /note="Description of Artificial Sequence: Synthetic peptide"
WLVKGRG

SEQ ID NO: 56 - protein, 6 aa, Artificial Sequence
  source: /note="Description of Artificial Sequence: Synthetic peptide"
LVKGRG

SEQ ID NO: 57 - protein, 31 aa, Artificial Sequence
  source: /note="Description of Artificial Sequence: Synthetic polypeptide"
  MOD_RES (10)..(10): (R)-alpha-(7'-octenyl)alanine or (R)-alpha-(4'-pentenyl)alanine
  MOD_RES (17)..(17): alpha,alpha-Bis(4'-pentenyl)glycine or alpha,alpha-Bis (7'-octenyl)glycine
  MOD_RES (24)..(24): (S)-alpha-(7'-octenyl)alanine or (S)-alpha-(4'-pentenyl)alanine
HGEGTFTSDXSSYLEGXAAKEFIXWLVKGRG

SEQ ID NO: 58 - protein, 31 aa, Artificial Sequence
  source: /note="Description of Artificial Sequence: Synthetic polypeptide"
  MOD_RES (2)..(2): 2-aminoisobutyric acid
  MOD_RES (10)..(10): (R)-alpha-(7'-octenyl)alanine or (R)-alpha-(4'-pentenyl)alanine
  MOD_RES (17)..(17): alpha,alpha-Bis(4'-pentenyl)glycine or alpha,alpha-Bis (7'-octenyl)glycine
  MOD_RES (24)..(24): (S)-alpha-(7'-octenyl)alanine or (S)-alpha-(4'-pentenyl)alanine
HXEGTFTSDXSSYLEGXAAKEFIXWLVKGRG

SEQ ID NO: 59 - protein, 31 aa, Artificial Sequence
  source: /note="Description of Artificial Sequence: Synthetic polypeptide"
  MOD_RES (11)..(11): (R)-alpha-(7'-octenyl)alanine or (R)-alpha-(4'-pentenyl)alanine
  MOD_RES (18)..(18): alpha,alpha-Bis(4'-pentenyl)glycine or alpha,alpha-Bis (7'-octenyl)glycine
  MOD_RES (25)..(25): (S)-alpha-(7'-octenyl)alanine or (S)-alpha-(4'-pentenyl)alanine
HGEGTFTSDVXSYLEGQXAKEFIAXLVKGRG

SEQ ID NO: 60 - protein, 31 aa, Artificial Sequence
  source: /note="Description of Artificial Sequence: Synthetic polypeptide"
  MOD_RES (2)..(2): 2-aminoisobutyric acid
  MOD_RES (11)..(11): (R)-alpha-(7'-octenyl)alanine or (R)-alpha-(4'-pentenyl)alanine
  MOD_RES (18)..(18): alpha,alpha-Bis(4'-pentenyl)glycine or alpha,alpha-Bis (7'-octenyl)glycine
  MOD_RES (25)..(25): (S)-alpha-(7'-octenyl)alanine or (S)-alpha-(4'-pentenyl)alanine
HXEGTFTSDVXSYLEGQXAKEFIAXLVKGRG

SEQ ID NO: 61 - protein, 30 aa, Artificial Sequence; identical to SEQ ID NO: 40
  source: /note="Description of Artificial Sequence: Synthetic polypeptide"
  MOD_RES (2)..(2): 2-aminoisobutyric acid
  MOD_RES (10)..(10): Stapling amino acid
  MOD_RES (17)..(17): Stapling amino acid
  MOD_RES (24)..(24): Stapling amino acid

SEQ ID NO: 62 - protein, 30 aa, Artificial Sequence; identical to SEQ ID NO: 33
  source: /note="Description of Artificial Sequence: Synthetic polypeptide"
  MOD_RES (10)..(10): Stapling amino acid
  MOD_RES (17)..(17): Stapling amino acid
  MOD_RES (24)..(24): Stapling amino acid

SEQ ID NO: 63 - protein, 31 aa, Artificial Sequence; identical to SEQ ID NO: 58
  source: /note="Description of Artificial Sequence: Synthetic polypeptide"
  MOD_RES (2)..(2): 2-aminoisobutyric acid
  MOD_RES (10)..(10): Stapling amino acid
  MOD_RES (17)..(17): Stapling amino acid
  MOD_RES (24)..(24): Stapling amino acid

SEQ ID NO: 64 - protein, 31 aa, Artificial Sequence; identical to SEQ ID NO: 57
  source: /note="Description of Artificial Sequence: Synthetic polypeptide"
  MOD_RES (10)..(10): Stapling amino acid
  MOD_RES (17)..(17): Stapling amino acid
  MOD_RES (24)..(24): Stapling amino acid

SEQ ID NO: 65 - protein, 30 aa, Artificial Sequence; identical to SEQ ID NO: 41
  source: /note="Description of Artificial Sequence: Synthetic polypeptide"
  MOD_RES (2)..(2): 2-aminoisobutyric acid
  MOD_RES (11)..(11): Stapling amino acid
  MOD_RES (18)..(18): Stapling amino acid
  MOD_RES (25)..(25): Stapling amino acid

SEQ ID NO: 66 - protein, 30 aa, Artificial Sequence; identical to SEQ ID NO: 34
  source: /note="Description of Artificial Sequence: Synthetic polypeptide"
  MOD_RES (11)..(11): Stapling amino acid
  MOD_RES (18)..(18): Stapling amino acid
  MOD_RES (25)..(25): Stapling amino acid

SEQ ID NO: 67 - protein, 31 aa, Artificial Sequence; identical to SEQ ID NO: 60
  source: /note="Description of Artificial Sequence: Synthetic polypeptide"
  MOD_RES (2)..(2): 2-aminoisobutyric acid
  MOD_RES (11)..(11): Stapling amino acid
  MOD_RES (18)..(18): Stapling amino acid
  MOD_RES (25)..(25): Stapling amino acid

SEQ ID NO: 68 - protein, 31 aa, Artificial Sequence; identical to SEQ ID NO: 59
  source: /note="Description of Artificial Sequence: Synthetic polypeptide"
  MOD_RES (11)..(11): Stapling amino acid
  MOD_RES (18)..(18): Stapling amino acid
  MOD_RES (25)..(25): Stapling amino acid

SEQ ID NO: 69 - protein, 31 aa, Artificial Sequence
  source: /note="Description of Artificial Sequence: Synthetic polypeptide"
HGEGTFTSDVSSYLEGQAAKEFIAWLVKGRG

SEQ ID NO: 70 - protein, 31 aa, Artificial Sequence
  source: /note="Description of Artificial Sequence: Synthetic polypeptide"
  MOD_RES (2)..(2): 2-aminoisobutyric acid
HXEGTFTSDVSSYLEGQAAKEFIAWLVKGRG

SEQ ID NO: 71 - protein, 30 aa, Artificial Sequence; identical to SEQ ID NO: 42
  source: /note="Description of Artificial Sequence: Synthetic polypeptide"
  MOD_RES (2)..(2): 2-aminoisobutyric acid
  MOD_RES (12)..(12): Stapling amino acid
  MOD_RES (19)..(19): Stapling amino acid
  MOD_RES (26)..(26): Stapling amino acid

SEQ ID NO: 72 - protein, 30 aa, Artificial Sequence; identical to SEQ ID NO: 42
  source: /note="Description of Artificial Sequence: Synthetic polypeptide"
  MOD_RES (2)..(2): 2-aminoisobutyric acid
  MOD_RES (12)..(12): (R)-alpha-(7'-octenyl)alanine or (R)-alpha-(4'-pentenyl)alanine
  MOD_RES (19)..(19): alpha,alpha-Bis(4'-pentenyl)glycine or alpha,alpha-Bis (7'-octenyl)glycine
  MOD_RES (26)..(26): (S)-alpha-(7'-octenyl)alanine or (S)-alpha-(4'-pentenyl)alanine

SEQ ID NO: 73 - protein, 30 aa, Artificial Sequence; identical to SEQ ID NO: 35
  source: /note="Description of Artificial Sequence: Synthetic polypeptide"
  MOD_RES (12)..(12): Stapling amino acid
  MOD_RES (19)..(19): Stapling amino acid
  MOD_RES (26)..(26): Stapling amino acid

SEQ ID NO: 74 - protein, 30 aa, Artificial Sequence; identical to SEQ ID NO: 35
  source: /note="Description of Artificial Sequence: Synthetic polypeptide"
  MOD_RES (12)..(12): (R)-alpha-(7'-octenyl)alanine or (R)-alpha-(4'-pentenyl)alanine
  MOD_RES (19)..(19): alpha,alpha-Bis(4'-pentenyl)glycine or alpha,alpha-Bis (7'-octenyl)glycine
  MOD_RES (26)..(26): (S)-alpha-(7'-octenyl)alanine or (S)-alpha-(4'-pentenyl)alanine

SEQ ID NO: 75 - protein, 31 aa, Artificial Sequence
  source: /note="Description of Artificial Sequence: Synthetic polypeptide"
  MOD_RES (2)..(2): 2-aminoisobutyric acid
  MOD_RES (12)..(12): Stapling amino acid
  MOD_RES (19)..(19): Stapling amino acid
  MOD_RES (26)..(26): Stapling amino acid
HXEGTFTSDVSXYLEGQAXKEFIAWXVKGRG

SEQ ID NO: 76 - protein, 31 aa, Artificial Sequence; identical to SEQ ID NO: 75
  source: /note="Description of Artificial Sequence: Synthetic polypeptide"
  MOD_RES (2)..(2): 2-aminoisobutyric acid
  MOD_RES (12)..(12): (R)-alpha-(7'-octenyl)alanine or (R)-alpha-(4'-pentenyl)alanine
  MOD_RES (19)..(19): alpha,alpha-Bis(4'-pentenyl)glycine or alpha,alpha-Bis (7'-octenyl)glycine
  MOD_RES (26)..(26): (S)-alpha-(7'-octenyl)alanine or (S)-alpha-(4'-pentenyl)alanine

SEQ ID NO: 77 - protein, 31 aa, Artificial Sequence
  source: /note="Description of Artificial Sequence: Synthetic polypeptide"
  MOD_RES (12)..(12): Stapling amino acid
  MOD_RES (19)..(19): Stapling amino acid
  MOD_RES (26)..(26): Stapling amino acid
HGEGTFTSDVSXYLEGQAXKEFIAWXVKGRG

SEQ ID NO: 78 - protein, 31 aa, Artificial Sequence; identical to SEQ ID NO: 77
  source: /note="Description of Artificial Sequence: Synthetic polypeptide"
  MOD_RES (12)..(12): (R)-alpha-(7'-octenyl)alanine or (R)-alpha-(4'-pentenyl)alanine
  MOD_RES (19)..(19): alpha,alpha-Bis(4'-pentenyl)glycine or alpha,alpha-Bis (7'-octenyl)glycine
  MOD_RES (26)..(26): (S)-alpha-(7'-octenyl)alanine or (S)-alpha-(4'-pentenyl)alanine

SEQ ID NO: 79 - protein, 30 aa, Artificial Sequence; identical to SEQ ID NO: 39
  source: /note="Description of Artificial Sequence: Synthetic polypeptide"
  MOD_RES (2)..(2): 2-aminoisobutyric acid
  MOD_RES (6)..(6): Stapling amino acid
  MOD_RES (13)..(13): Stapling amino acid
  MOD_RES (20)..(20): Stapling amino acid

SEQ ID NO: 80 - protein, 30 aa, Artificial Sequence; identical to SEQ ID NO: 39
  source: /note="Description of Artificial Sequence: Synthetic polypeptide"
  MOD_RES (2)..(2): 2-aminoisobutyric acid
  MOD_RES (6)..(6): (R)-alpha-(7'-octenyl)alanine or (R)-alpha-(4'-pentenyl)alanine
  MOD_RES (13)..(13): alpha,alpha-Bis(4'-pentenyl)glycine or alpha,alpha-Bis (7'-octenyl)glycine
  MOD_RES (20)..(20): (S)-alpha-(7'-octenyl)alanine or (S)-alpha-(4'-pentenyl)alanine

SEQ ID NO: 81 - protein, 30 aa, Artificial Sequence; identical to SEQ ID NO: 32
  source: /note="Description of Artificial Sequence: Synthetic polypeptide"
  MOD_RES (6)..(6): Stapling amino acid
  MOD_RES (13)..(13): Stapling amino acid
  MOD_RES (20)..(20): Stapling amino acid

SEQ ID NO: 82 - protein, 30 aa, Artificial Sequence; identical to SEQ ID NO: 32
  source: /note="Description of Artificial Sequence: Synthetic polypeptide"
  MOD_RES (6)..(6): (R)-alpha-(7'-octenyl)alanine or (R)-alpha-(4'-pentenyl)alanine
  MOD_RES (13)..(13): alpha,alpha-Bis(4'-pentenyl)glycine or alpha,alpha-Bis (7'-octenyl)glycine
  MOD_RES (20)..(20): (S)-alpha-(7'-octenyl)alanine or (S)-alpha-(4'-pentenyl)alanine

SEQ ID NO: 83 - protein, 31 aa, Artificial Sequence
  source: /note="Description of Artificial Sequence: Synthetic polypeptide"
  MOD_RES (2)..(2): 2-aminoisobutyric acid
  MOD_RES (6)..(6): Stapling amino acid
  MOD_RES (13)..(13): Stapling amino acid
  MOD_RES (20)..(20): Stapling amino acid
HXEGTXTSDVSSXLEGQAAXEFIAWLVKGRG

SEQ ID NO: 84 - protein, 31 aa, Artificial Sequence; identical to SEQ ID NO: 83
  source: /note="Description of Artificial Sequence: Synthetic polypeptide"
  MOD_RES (2)..(2): 2-aminoisobutyric acid
  MOD_RES (6)..(6): (R)-alpha-(7'-octenyl)alanine or (R)-alpha-(4'-pentenyl)alanine
  MOD_RES (13)..(13): alpha,alpha-Bis(4'-pentenyl)glycine or alpha,alpha-Bis (7'-octenyl)glycine
  MOD_RES (20)..(20): (S)-alpha-(7'-octenyl)alanine or (S)-alpha-(4'-pentenyl)alanine

SEQ ID NO: 85 - protein, 31 aa, Artificial Sequence
  source: /note="Description of Artificial Sequence: Synthetic polypeptide"
  MOD_RES (6)..(6): Stapling amino acid
  MOD_RES (13)..(13): Stapling amino acid
  MOD_RES (20)..(20): Stapling amino acid
HGEGTXTSDVSSXLEGQAAXEFIAWLVKGRG

SEQ ID NO: 86 - protein, 31 aa, Artificial Sequence; identical to SEQ ID NO: 85
  source: /note="Description of Artificial Sequence: Synthetic polypeptide"
  MOD_RES (6)..(6): (R)-alpha-(7'-octenyl)alanine or (R)-alpha-(4'-pentenyl)alanine
  MOD_RES (13)..(13): alpha,alpha-Bis(4'-pentenyl)glycine or alpha,alpha-Bis (7'-octenyl)glycine
  MOD_RES (20)..(20): (S)-alpha-(7'-octenyl)alanine or (S)-alpha-(4'-pentenyl)alanine

SEQ ID NO: 87 - protein, 6 aa, Artificial Sequence
  source: /note="Description of Artificial Sequence: Synthetic peptide"
TSDVSS

SEQ ID NO: 88 - protein, 6 aa, Artificial Sequence
  source: /note="Description of Artificial Sequence: Synthetic peptide"
LEGQAA

SEQ ID NO: 89 - protein, 6 aa, Artificial Sequence
  source: /note="Description of Artificial Sequence: Synthetic peptide"
YLEGQA

SEQ ID NO: 90 - protein, 6 aa, Artificial Sequence
  source: /note="Description of Artificial Sequence: Synthetic peptide"
KEFIAW

SEQ ID NO: 91 - protein, 5 aa, Artificial Sequence
  source: /note="Description of Artificial Sequence: Synthetic peptide"
HGEGT

SEQ ID NO: 92 - protein, 5 aa, Artificial Sequence
  source: /note="Description of Artificial Sequence: Synthetic peptide"
  MOD_RES (2)..(2): 2-aminoisobutyric acid
HXEGT

SEQ ID NO: 93 - protein, 10 aa, Artificial Sequence
  source: /note="Description of Artificial Sequence: Synthetic peptide"
EFIAWLVKGR

SEQ ID NO: 94 - protein, 11 aa, Artificial Sequence
  source: /note="Description of Artificial Sequence: Synthetic peptide"
EFIAWLVKGRG

SEQ ID NO: 95 - protein, 11 aa, Artificial Sequence
  source: /note="Description of Artificial Sequence: Synthetic peptide"
HGEGTFTSDVS

SEQ ID NO: 96 - protein, 11 aa, Artificial Sequence
  source: /note="Description of Artificial Sequence: Synthetic peptide"
  MOD_RES (2)..(2): 2-aminoisobutyric acid
HXEGTFTSDVS

SEQ ID NO: 97 - protein, 4 aa, Artificial Sequence
  source: /note="Description of Artificial Sequence: Synthetic peptide"
VKGR

SEQ ID NO: 98 - protein, 5 aa, Artificial Sequence
  source: /note="Description of Artificial Sequence: Synthetic peptide"
VKGRG

SEQ ID NO: 99-105 - skipped (7 entries)

SEQ ID NO: 106 - protein, 30 aa, Artificial Sequence
  source: /note="Description of Artificial Sequence: Synthetic polypeptide"
  MOD_RES (2)..(2): 2-aminoisobutyric acid
  MOD_RES (10)..(10): R-octenyl alanine
  MOD_RES (17)..(17): S-pentenyl alanine
HXEGTFTSDXSSYLEGXAAKEFIAWLVKGR

SEQ ID NO: 107 - protein, 30 aa, Artificial Sequence
  source: /note="Description of Artificial Sequence: Synthetic polypeptide"
  MOD_RES (2)..(2): 2-aminoisobutyric acid
  MOD_RES (17)..(17): R-octenyl alanine
  MOD_RES (24)..(24): S-pentenyl alanine
HXEGTFTSDVSSYLEGXAAKEFIXWLVKGR

SEQ ID NO: 108 - protein, 10 aa, Artificial Sequence
  source: /note="Description of Artificial Sequence: Synthetic peptide"
  MOD_RES (1)..(1): N-(2-(1H-imidazol-5-yl)ethyl)-2,2-dimethyl-3-oxobutanyl
  MOD_RES (2)..(2): tetrazolyl-alanine
  MOD_RES (5)..(5): alpha-methyl ortho flurophenylalanine
  MOD_RES (9)..(9): 3-(4'-methoxy-2'-ethyl[1,1'biphenyl]-4-yl-L-Ala
  MOD_RES (10)..(10): 5-(3,5-dimethylphenyl)-L-norvaline
XXGTXTSDXX

SEQ ID NO: 109 - protein, 26 aa, Homo sapiens
GTFTSDVSSYLEGQAAKEFIAWLVKG

SEQ ID NO: 110 - protein, 21 aa, Homo sapiens
DVSSYLEGQAAKEFIAWLVKG